SEQUENCE LISTING (198 sequences)

SEQ ID NO: 1 - protein, 118 aa, Artificial Sequence [sequence omitted]

SEQ ID NO: 2 - protein, 8 aa, Artificial Sequence
  Description of Artificial Sequence Synthetic peptide
GFTFNNYA

SEQ ID NO: 3 - protein, 8 aa, Artificial Sequence
  Description of Artificial Sequence Synthetic peptide
ISGSGGTT

SEQ ID NO: 4 - protein, 11 aa, Artificial Sequence
  Description of Artificial Sequence Synthetic peptide
AKDSNWGNFDL

SEQ ID NO: 5 - protein, 447 aa, Artificial Sequence
  Description of Artificial Sequence Synthetic REGN727 heavy chain polypeptide
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 113 aa, Artificial Sequence [sequence omitted]

SEQ ID NO: 7 - protein, 12 aa, Artificial Sequence
  Description of Artificial Sequence Synthetic peptide
QSVLYRSNNRNF

SEQ ID NO: 8 - protein, 3 aa, Artificial Sequence
  Description of Artificial Sequence Synthetic peptide
WAS

SEQ ID NO: 9 - protein, 220 aa, Artificial Sequence
  Description of Artificial Sequence Synthetic REGN727 light chain polypeptide
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 9 aa, Artificial Sequence
  Description of Artificial Sequence Synthetic peptide
QQYYTTPYT

SEQ ID NO: 11 - protein, 127 aa, Artificial Sequence
  Description of Artificial Sequence Synthetic polypeptide
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 12 - protein, 8 aa, Artificial Sequence
  Description of Artificial Sequence Synthetic peptide
GFTFSSHW

SEQ ID NO: 13 - protein, 8 aa, Artificial Sequence
  Description of Artificial Sequence Synthetic peptide
INQDGSEK

SEQ ID NO: 14 - protein, 20 aa, Artificial Sequence
  Description of Artificial Sequence Synthetic peptide
ARDIVLMVYDMDYYYYGMDV

SEQ ID NO: 15 - protein, 112 aa, Artificial Sequence
  Description of Artificial Sequence Synthetic polypeptide
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK

SEQ ID NO: 16 - protein, 11 aa, Artificial Sequence
  Description of Artificial Sequence Synthetic peptide
QSLLHSNGNNY

SEQ ID NO: 17 - protein, 3 aa, Artificial Sequence
  Description of Artificial Sequence Synthetic peptide
LGS

SEQ ID NO: 18 - protein, 9 aa, Artificial Sequence
  Description of Artificial Sequence Synthetic peptide
MQTLQTPLT

SEQ ID NO: 19 - protein, 8 aa, Artificial Sequence
  Description of Artificial Sequence Synthetic peptide
GFTFSSHW

SEQ ID NO: 20 - protein, 8 aa, Artificial Sequence
  Description of Artificial Sequence Synthetic peptide
INQDGSEK

SEQ ID NO: 21 - protein, 20 aa, Artificial Sequence
  Description of Artificial Sequence Synthetic peptide
ARDIVLMVYDMDYYYYGMDV

SEQ ID NO: 22 - protein, 11 aa, Artificial Sequence
  Description of Artificial Sequence Synthetic peptide
QSLHHSNGNNY

SEQ ID NO: 23 - protein, 3 aa, Artificial Sequence
  Description of Artificial Sequence Synthetic peptide
LGS

SEQ ID NO: 24 - protein, 9 aa, Artificial Sequence
  Description of Artificial Sequence Synthetic peptide
MQTLQTPLT

SEQ ID NO: 25 - protein, 8 aa, Artificial Sequence
  Description of Artificial Sequence Synthetic peptide
GFTFSSHW

SEQ ID NO: 26 - protein, 8 aa, Artificial Sequence
  Description of Artificial Sequence Synthetic peptide
INQDGSEK

SEQ ID NO: 27 - protein, 20 aa, Artificial Sequence
  Description of Artificial Sequence Synthetic peptide
ARDIVLMVYHMDYYYYGMDV

SEQ ID NO: 28 - protein, 11 aa, Artificial Sequence
  Description of Artificial Sequence Synthetic peptide
QSLLHSNGNNY

SEQ ID NO: 29 - protein, 3 aa, Artificial Sequence
  Description of Artificial Sequence Synthetic peptide
LGS

SEQ ID NO: 30 - protein, 9 aa, Artificial Sequence
  Description of Artificial Sequence Synthetic peptide
MQTLQTPLT

SEQ ID NO: 31 - protein, 8 aa, Artificial Sequence
  Description of Artificial Sequence Synthetic peptide
GFTFSSHW

SEQ ID NO: 32 - protein, 8 aa, Artificial Sequence
  Description of Artificial Sequence Synthetic peptide
INQDGSEK

SEQ ID NO: 33 - protein, 20 aa, Artificial Sequence
  Description of Artificial Sequence Synthetic peptide
ARDIVLMVYHMDYYYYGMDV

SEQ ID NO: 34 - protein, 11 aa, Artificial Sequence
  Description of Artificial Sequence Synthetic peptide
QSLHHSNGNNY

SEQ ID NO: 35 - protein, 3 aa, Artificial Sequence
  Description of Artificial Sequence Synthetic peptide
LGS

SEQ ID NO: 36 - protein, 9 aa, Artificial Sequence
  Description of Artificial Sequence Synthetic peptide
MQTLQTPLT

SEQ ID NO: 37 - protein, 131 aa, Artificial Sequence
  Description of Artificial Sequence Synthetic VH; m2CX1D05 polypeptide
QVQLVQSGAEVKKPGSSVKVSCKASGGTFNSHAISWVRQAPGQGLEWMGGINPILGIANYAQKFQGRVTITADESTSTAYMELSSLRSEDTAVYYCARHYEIQIGRYGMNVYYLMYRFASWGQGTLVTVSS

SEQ ID NO: 38 - protein, 10 aa, Artificial Sequence
  Description of Artificial Sequence Synthetic VH CDR1; m2CX1D05 peptide
GGTFNSHAIS

SEQ ID NO: 39 - protein, 20 aa, Artificial Sequence
  Description of Artificial Sequence Synthetic VH CDR2; m2CX1D05 peptide
WMGGINPILGIANYAQKFQG

SEQ ID NO: 40 - protein, 22 aa, Artificial Sequence
  Description of Artificial Sequence Synthetic VH CDR3; m2CX1D05 peptide
HYEIQIGRYGMNVYYLMYRFAS

SEQ ID NO: 41 - protein, 213 aa, Artificial Sequence
  Description of Artificial Sequence Synthetic LC; m2CX1D05 polypeptide
DIQMTQSPSSLSASVGDRVTITCRASQGIRSALNWYQQKPGKAPKLLIYNGSTLQSGVPSRFSGSGSGTDFTLTISSLQPEDFAVYYCQQFDGDPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 42 - protein, 11 aa, Artificial Sequence
  Description of Artificial Sequence Synthetic VL CDR 1; m2CX1D05 peptide
RASQGIRSALN

SEQ ID NO: 43 - protein, 11 aa, Artificial Sequence
  Description of Artificial Sequence Synthetic VL CDR2; m2CX1D05 peptide
LLIYNGSTLQS

SEQ ID NO: 44 - protein, 7 aa, Artificial Sequence
  Description of Artificial Sequence Synthetic VL CDR3; m2CX1D05 peptide
QQFDGDP

SEQ ID NO: 45 - protein, 119 aa, Artificial Sequence
  Description of Artificial Sequence Synthetic VH; 1B20 polypeptide
QVQLVQSGAEVKKPGESLKISCKGSGYSFTNYWISWVRQMPGKGLEWMGIIYPGDSYTNYSPSFQGQVTISADKSISTAYLQWSSLKASDTAMYYCARDYWYKPLFDIWGQGTLVTVSS

SEQ ID NO: 46 - protein, 10 aa, Artificial Sequence
  Description of Artificial Sequence Synthetic VH CDR1; 1B20 peptide
GYSFTNYWIS

SEQ ID NO: 47 - protein, 20 aa, Artificial Sequence
  Description of Artificial Sequence Synthetic VH CDR2; 1B20 peptide
WMGIIYPGDSYTNYSPSFQG

SEQ ID NO: 48 - protein, 10 aa, Artificial Sequence
  Description of Artificial Sequence Synthetic VH CDR3; 1B20 peptide
DYWYKPLFDI

SEQ ID NO: 49 - protein, 220 aa, Artificial Sequence
  Description of Artificial Sequence Synthetic LC; 1B20 polypeptide
DIVMTQSPDSLAVSLGERATINCRSSQSVLYSSNNKNYLAWYQQKPGQPPKLLIYWASTRESGVPDRFSGSGSGTDFTLTISSLQAEDVAVYYCQQYSSFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 50 - protein, 17 aa, Artificial Sequence
  Description of Artificial Sequence Synthetic VL CDR1; 1B20 peptide
RSSQSVLYSSNNKNYLA

SEQ ID NO: 51 - protein, 11 aa, Artificial Sequence
  Description of Artificial Sequence Synthetic VL CDR2; 1B20 peptide
LLIYWASTRES

SEQ ID NO: 52 - protein, 8 aa, Artificial Sequence
  Description of Artificial Sequence Synthetic VL CDR3; 1B20 peptide
QQYSSFPI

SEQ ID NO: 53 - protein, 120 aa, Artificial Sequence
  Description of Artificial Sequence Synthetic variable heavy antibody region polypeptide
EVQLLESGGGLVQPGGSLRLSCKASGYTFSSYGMYWVRQAPGKGLEWIGWIDPGSGGTKYNEKFKGKATISRDNSKNTLYLQMNSLRAEDTAVYYCARERYGYYFDYWGQGTLVTVSSAS

SEQ ID NO: 54 - protein, 16 aa, Artificial Sequence
  Description of Artificial Sequence Synthetic AX132 heavy chain CDR1 antibody region peptide
KASGYTFSSYGMYWVR

SEQ ID NO: 55 - protein, 23 aa, Artificial Sequence
  Description of Artificial Sequence Synthetic AX132 heavy chain CDR2 antibody region peptide
WIGWIDPGSGGTKYNEKFKGKAT

SEQ ID NO: 56 - protein, 15 aa, Artificial Sequence
  Description of Artificial Sequence Synthetic AX132 heavy chain CDR3 antibody region peptide
CARERYGYYFDYWGQ

SEQ ID NO: 57 - protein, 108 aa, Artificial Sequence
  Description of Artificial Sequence Synthetic variable light antibody region polypeptide
EIVLTQSPATLSLSPGERATITCRASQYVGSYLNWYQQKPGQAPRLLIYDASNRATGIPARFSGSGSGTDFTLTISSLEPEDFAVYYCQVWDSSPPVVFGGGTKVEIK

SEQ ID NO: 58 - protein, 17 aa, Artificial Sequence
  Description of Artificial Sequence Synthetic AX213 and AX132 light chain CDR1 antibody region peptide
ITCRASQYVGSYLNWYQ

SEQ ID NO: 59 - protein, 13 aa, Artificial Sequence
  Description of Artificial Sequence Synthetic AX213 and AX132 light chain CDR2 antibody region peptide
LIYDASNRATGIP

SEQ ID NO: 60 - protein, 16 aa, Artificial Sequence
  Description of Artificial Sequence Synthetic AX132 & AX213 light chain CDR3 antibody region peptide
YYCQVWDSSPPVVFGG

SEQ ID NO: 61 - protein, 120 aa, Artificial Sequence
  Description of Artificial Sequence Synthetic variable heavy antibody region polypeptide
EVQLLESGGGLVQPGGSLRLSCKASGYTFSRYGINWVRQAPGKGLEWIGRIDPGNGGTRYNEKFKGKATISRDNSKNTLYLQMNSLRAEDTAVYYCARANDGYSFDYWGQGTLVTVSSAS

SEQ ID NO: 62 - protein, 16 aa, Artificial Sequence
  Description of Artificial Sequence Synthetic AX213 heavy chain CDR1 antibody region peptide
KASGYTFSRYGINWVR

SEQ ID NO: 63 - protein, 23 aa, Artificial Sequence
  Description of Artificial Sequence Synthetic AX213 heavy chain CDR2 antibody region peptide
WIGRIDPGNGGTRYNEKFKGKAT

SEQ ID NO: 64 - protein, 15 aa, Artificial Sequence
  Description of Artificial Sequence Synthetic AX213 heavy chain CDR3 antibody region peptide
CARANDGYSFDYWGQ

SEQ ID NO: 65 - protein, 108 aa, Artificial Sequence; identical to SEQ ID NO: 57
  Description of Artificial Sequence Synthetic variable light antibody region polypeptide

SEQ ID NO: 66 - protein, 17 aa, Artificial Sequence
  Description of Artificial Sequence Synthetic AX213 and AX132 light chain CDR1 antibody region peptide
ITCRASQYVGSYLNWYQ

SEQ ID NO: 67 - protein, 13 aa, Artificial Sequence
  Description of Artificial Sequence Synthetic AX213 and AX132 light chain CDR2 antibody region peptide
LIYDASNRATGIP

SEQ ID NO: 68 - protein, 16 aa, Artificial Sequence
  Description of Artificial Sequence Synthetic AX132 & AX213 light chain CDR3 antibody region peptide
YYCQVWDSSPPVVFGG

SEQ ID NO: 69 - protein, 119 aa, Artificial Sequence
  Description of Artificial Sequence Synthetic AX1 VH antibody sequence polypeptide
EVQLLESGGGLVQPGGSLRLSCKASGFTFTSYYMHWVRQAPGKGLEWIGRINPDSGSTKYNEKFKGRATISRDNSKNTLYLQMNSLRAEDTAVYYCARGGRLSWDFDVWGQGTLVTVSS

SEQ ID NO: 70 - protein, 16 aa, Artificial Sequence
  Description of Artificial Sequence Synthetic AX1 VH CDR1 antibody sequence peptide
KASGFTFTSYYMHWVR

SEQ ID NO: 71 - protein, 23 aa, Artificial Sequence
  Description of Artificial Sequence Synthetic AX1 VH CDR2 antibody sequence peptide
WIGRINPDSGSTKYNEKFKGRAT

SEQ ID NO: 72 - protein, 16 aa, Artificial Sequence
  Description of Artificial Sequence Synthetic AX1 VH CDR3 antibody sequence peptide
CARGGRLSWDFDVWGQ

SEQ ID NO: 73 - protein, 109 aa, Artificial Sequence
  Description of Artificial Sequence Synthetic AX1 VL antibody sequence polypeptide
DIQMTQSPSSLSASVGDRVTITCRASQDISRYLAWYQQKPGKAPKLLIYAASSLQSGVPSRFSGSGSGTDFTLTISSLQPEDFATYYCAAYDYSLGGYVFGDGTKVEIK

SEQ ID NO: 74 - protein, 11 aa, Artificial Sequence
  Description of Artificial Sequence Synthetic AX1 VL CDR1 antibody sequence peptide
RASQDISRYLA

SEQ ID NO: 75 - protein, 7 aa, Artificial Sequence
  Description of Artificial Sequence Synthetic AX1 AX9 AX189 VL CDR2 antibody sequence peptide
AASSLQS

SEQ ID NO: 76 - protein, 11 aa, Artificial Sequence
  Description of Artificial Sequence Synthetic AX1 VL CDR3 antibody sequence peptide
AAYDYSLGGYV

SEQ ID NO: 77 - protein, 121 aa, Artificial Sequence
  Description of Artificial Sequence Synthetic AX9 AX189 VH antibody sequence polypeptide
EVQLLESGGGLVQPGGSLRLSCKASGYTFSSYWMHWVRQAPGKGLEWIGRIDPYNGGTKYNEKFKGKATISRDNSKNTLYLQMNSLRAEDTAVYYCARYGYYLGSYAMDYWGQGTLVTVSS

SEQ ID NO: 78 - protein, 16 aa, Artificial Sequence
  Description of Artificial Sequence Synthetic AX9 AX189 VH CDR1 antibody sequence peptide
KASGYTFSSYWMHWVR

SEQ ID NO: 79 - protein, 23 aa, Artificial Sequence
  Description of Artificial Sequence Synthetic AX9 AX189 VH CDR2 antibody sequence peptide
WIGRIDPYNGGTKYNEKFKGKAT

SEQ ID NO: 80 - protein, 18 aa, Artificial Sequence
  Description of Artificial Sequence Synthetic AX9 AX189 VH CDR3 antibody sequence peptide
CARYGYYLGSYAMDYWGQ

SEQ ID NO: 81 - protein, 109 aa, Artificial Sequence
  Description of Artificial Sequence Synthetic AX189 VL antibody sequence polypeptide
DIQMTQSPSSLSASVGDRVTITCRASQDVSRYLTWYQQKPGKAPKLLIYAASSLQSGVPSRFSGSGSGTDFTLTISSLQPEDFATYYCQAYDYSLSGYVFGGGTKVEIK

SEQ ID NO: 82 - protein, 11 aa, Artificial Sequence
  Description of Artificial Sequence Synthetic AX189 VL CDR1 antibody sequence peptide
RASQDVSRYLT

SEQ ID NO: 83 - protein, 7 aa, Artificial Sequence
  Description of Artificial Sequence Synthetic AX1 AX9 AX189 VL CDR2 antibody sequence peptide
AASSLQS

SEQ ID NO: 84 - protein, 11 aa, Artificial Sequence
  Description of Artificial Sequence Synthetic AX189 VL CDR3 antibody sequence peptide
QAYDYSLSGYV

SEQ ID NO: 85 - protein, 115 aa, Homo sapiens
QVQLVQSGAEVKKPGASVKVSCKASGYTLTSYGISWVRQAPGQGLEWMGWVSFYNGNTNYAQKLQGRGTMTTDPSTSTAYMELRSLRSDDTAVYYCARGYGMDVWGQGTTVTVSS

SEQ ID NO: 86 - protein, 5 aa, Homo sapiens
SYGIS

SEQ ID NO: 87 - protein, 17 aa, Homo sapiens
WVSFYNGNTNYAQKLQG

SEQ ID NO: 88 - protein, 6 aa, Homo sapiens
GYGMDV

SEQ ID NO: 89 - protein, 109 aa, Homo sapiens
QSALTQPASVSGSPGQSITISCTGTSSDVGGYNSVSWYQQHPGKAPKLMIYEVSNRPSGVSNRFSGSKSGNTASLTISGLQAEDEADYYCNSYTSTSMVFGGGTKLTVL

SEQ ID NO: 90 - protein, 14 aa, Homo sapiens
TGTSSDVGGYNSVS

SEQ ID NO: 91 - protein, 7 aa, Homo sapiens
EVSNRPS

SEQ ID NO: 92 - protein, 9 aa, Homo sapiens
NSYTSTSMV

SEQ ID NO: 93 - protein, 123 aa, Homo sapiens
EVQLVESGGGLVKPGGSLRLSCAASGFTFSSYSMNWVRQAPGKGLEWVSSISSSSSYISYADSVKGRFTISRDNAKNSLYLQMNSLRAEDTAVYFCARDYDFWSAYYDAFDVWGQGTMVTVSS

SEQ ID NO: 94 - protein, 10 aa, Homo sapiens
GFTFSSYSMN

SEQ ID NO: 95 - protein, 17 aa, Homo sapiens
SISSSSSYISYADSVKG

SEQ ID NO: 96 - protein, 14 aa, Homo sapiens
DYDFWSAYYDAFDV

SEQ ID NO: 97 - protein, 111 aa, Homo sapiens
QSVLTQPPSVSGAPGQRVTISCTGSSSNIGAGYDVHWYQQLPGTAPKLLISGNSNRPSGVPDRFSGSKSGTSASLAITGLQAEDEADYYCQSYDSSLSGSVFGGGTKLTVL

SEQ ID NO: 98 - protein, 14 aa, Homo sapiens
TGSSSNIGAGYDVH

SEQ ID NO: 99 - protein, 7 aa, Homo sapiens
GNSNRPS

SEQ ID NO: 100 - protein, 11 aa, Homo sapiens
QSYDSSLSGSV

SEQ ID NO: 101 - protein, 114 aa, Homo sapiens
QVQLVESGGGVAQPGRSLRLSCAASGFTFSSYGMHWVRQAPGKGLEWVAVIYYDGINKHYADSVKGRFTISRDNSKNTLYLQMNSLRAEDTAVYYCARDRGLDWGQGTLVTVSS

SEQ ID NO: 102 - protein, 10 aa, Homo sapiens
GFTFSSYGMH

SEQ ID NO: 103 - protein, 17 aa, Homo sapiens
VIYYDGINKHYADSVKG

SEQ ID NO: 104 - protein, 5 aa, Homo sapiens
DRGLD

SEQ ID NO: 105 - protein, 113 aa, Homo sapiens
DIVMTQSPDSLAVSLGERATINCKSSQSVLYSSNSKNYLVWYQQKPGQPPKLLIYWASTRESGVPDRFSGSGSGTDFTLTISSLQAEDVAVYYCQQYYSTPWTFGQGTKVEIK

SEQ ID NO: 106 - protein, 17 aa, Homo sapiens
KSSQSVLYSSNSKNYLV

SEQ ID NO: 107 - protein, 7 aa, Homo sapiens
WASTRES

SEQ ID NO: 108 - protein, 9 aa, Homo sapiens
QQYYSTPWT

SEQ ID NO: 109 - protein, 118 aa, Artificial Sequence
  Description of Artificial Sequence Synthetic anti-PCSK9 monoclonal antibody pJG04 (clones LGT-209 and LGT-210) Vh heavy chain variable region (FR1-FR4) polypeptide
QVQLVQSGAEVKKPGASVKVSCKASGYTFSTMYMSWVRQAPGQGLEWMGRIDPANEHTNYAQKFQGRVTMTRDTSISTAYMELSRLTSDDTAVYYCARSYYYYNMDYWGQGTLVTVSS

SEQ ID NO: 110 - protein, 5 aa, Artificial Sequence
  Description of Artificial Sequence Synthetic anti-PCSK9 monoclonal antibody clones LGT-209, LGT-210 and LGT-211 heavy chain CDR1 peptide
TMYMS

SEQ ID NO: 111 - protein, 17 aa, Artificial Sequence
  Description of Artificial Sequence Synthetic anti-PCSK9 monoclonal antibody clones LGT-209, LGT-210 and LGT-211 heavy chain CDR2 peptide
RIDPANEHTNYAQKFQG

SEQ ID NO: 112 - protein, 9 aa, Artificial Sequence
  Description of Artificial Sequence Synthetic anti-PCSK9 monoclonal antibody pJG04(clones LGT-209 and LGT-210) Vh heavy chain complementarity determining region 3 (CDR3) peptide
SYYYYNMDY

SEQ ID NO: 113 - protein, 106 aa, Artificial Sequence
  Description of Artificial Sequence Synthetic anti-PCSK9 monoclonal antibody pJG10(clones LGT-209 and LGT-211) Vk light chain variable region (FR1-FR4) polypeptide
QIVLTQSPATLSVSPGERATLSCRASQSVSYMHWYQQKPGQAPRLLIYGVFRRATGIPDRFSGSGSGTDFTLTIGRLEPEDFAVYYCLQWSSDPPTFGQGTKLEIK

SEQ ID NO: 114 - protein, 10 aa, Artificial Sequence
  Description of Artificial Sequence Synthetic anti-PCSK9 monoclonal antibody clones LGT-209, LGT-210 and LGT-211 light chain CDR1 peptide
RASQSVSYMH

SEQ ID NO: 115 - protein, 7 aa, Artificial Sequence
  Description of Artificial Sequence Synthetic anti-PCSK9 monoclonal antibody clones LGT-209, LGT-210 and LGT-211 light chain CDR1 peptide
GVFRRAT

SEQ ID NO: 116 - protein, 9 aa, Artificial Sequence
  Description of Artificial Sequence Synthetic mouse anti-PCSK9 monoclonal antibody LFU720 and anti-PCSK9 monoclonal antibody clones LGT-209, LGT-210 and LGT-211 light chain CDR3 peptide
LQWSSDPPT

SEQ ID NO: 117 - protein, 118 aa, Homo sapiens
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 118 - protein, 7 aa, Artificial Sequence
  Description of Artificial Sequence Synthetic peptide
GYTFTSY

SEQ ID NO: 119 - protein, 6 aa, Artificial Sequence
  Description of Artificial Sequence Synthetic peptide
SPFGGR

SEQ ID NO: 120 - protein, 9 aa, Artificial Sequence
  Description of Artificial Sequence Synthetic variable heavy chain CDR peptide
ERPLYASDL

SEQ ID NO: 121 - protein, 107 aa, Homo sapiens
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 122 - protein, 11 aa, Artificial Sequence
  Description of Artificial Sequence Synthetic variable light chain CDR peptide
RASQGISSALA

SEQ ID NO: 123 - protein, 7 aa, Artificial Sequence
  Description of Artificial Sequence Synthetic variable light chain CDR peptide
SASYRYT

SEQ ID NO: 124 - protein, 9 aa, Artificial Sequence
  Description of Artificial Sequence Synthetic variable light chain CDR peptide
QQRYSLWRT

SEQ ID NO: 125 - protein, 118 aa, Homo sapiens
QVQLVQSGAEVKKPGASVKVSCKASGYTFTSYYMHWVRQAPGQGLEWMGEIHPSGGRTNYNEKFKSRVTMTRDTSTSTVYMELSSLRSEDTAVYYCARERPLYAMDYWGQGTTVTVSS

SEQ ID NO: 126 - protein, 10 aa, Homo sapiens
GYTFTSYYMH

SEQ ID NO: 127 - protein, 17 aa, Homo sapiens
EIHPSGGRTNYNEKFKS

SEQ ID NO: 128 - protein, 9 aa, Homo sapiens
ERPLYAMDY

SEQ ID NO: 129 - protein, 107 aa, Homo sapiens [sequence omitted]

SEQ ID NO: 130 - protein, 11 aa, Homo sapiens
KASQDVHTAVA

SEQ ID NO: 131 - protein, 7 aa, Homo sapiens
HASYRYT

SEQ ID NO: 132 - protein, 9 aa, Homo sapiens
QQRYSLWRT

SEQ ID NO: 133 - protein, 118 aa, Homo sapiens [sequence omitted]

SEQ ID NO: 134 - protein, 10 aa, Homo sapiens
GYTFTSYYMH

SEQ ID NO: 135 - protein, 17 aa, Homo sapiens
EIHPSGGRTNYNEKFKS

SEQ ID NO: 136 - protein, 9 aa, Homo sapiens
ERPLYASDL

SEQ ID NO: 137 - protein, 107 aa, Homo sapiens [sequence omitted]

SEQ ID NO: 138 - protein, 11 aa, Homo sapiens
KASQDVHTAVA

SEQ ID NO: 139 - protein, 7 aa, Homo sapiens
HASYRYT

SEQ ID NO: 140 - protein, 9 aa, Homo sapiens
QQRYSLWRT

SEQ ID NO: 141 - protein, 118 aa, Mus musculus
QVQLQQPGAELVKPGASVKLSCKASGYTFTSYWMHWVKQRPGQGLEWIGEINPSNGRTNYNEKFKSKATLTVDKSSSTAYMQLSSLTSEDSAVYYCARERPLYAMDYWGQGTSVTVSS

SEQ ID NO: 142 - protein, 7 aa, Mus musculus
GYTFTSY

SEQ ID NO: 143 - protein, 6 aa, Mus musculus
NPSNGR

SEQ ID NO: 144 - protein, 9 aa, Mus musculus
ERPLYAMDY

SEQ ID NO: 145 - protein, 108 aa, Mus musculus
DIVMTQSHKFMSTSVGDRVSITCKASQDVSTAVAWYQQKPGQSPKLLIYSASYRYTGVPDRFTGSGSGTDFTFTISSVQAEDLAVYYCQQRYSTPRTFGGGTKLEIKR

SEQ ID NO: 146 - protein, 11 aa, Mus musculus
KASQDVSTAVA

SEQ ID NO: 147 - protein, 7 aa, Artificial Sequence
  Description of Artificial Sequence Synthetic variable light chain CDR peptide
SASYRYT

SEQ ID NO: 148 - protein, 9 aa, Mus musculus
QQRYSTPRT

SEQ ID NO: 149 - protein, 115 aa, Mus musculus
EVQLQQSGPELVKPGASVKISCKASGYTFTDYYMNWVKQSHGKSLEWIGDINPNNGGTTYNQKFKGKATLTVDKSYSTAYMELRSLTSEDSAVYYCARWLLFAYWGQGTLVTVSA

SEQ ID NO: 150 - protein, 7 aa, Mus musculus
GYTFTDY

SEQ ID NO: 151 - protein, 6 aa, Mus musculus
NPNNGG

SEQ ID NO: 152 - protein, 6 aa, Mus musculus
WLLFAY

SEQ ID NO: 153 - protein, 108 aa, Mus musculus
DIVMTQSQKFMSTSVGDRVSVTCKASQNVGTNVAWYQQKPGQSPKALIYSASYRYSGVPDRFTGSGSGTDFTLTISNVLSEDLAEYFCQQFYSYPYTFGGGTKLEIKR

SEQ ID NO: 154 - protein, 11 aa, Mus musculus
KASQNVGTNVA

SEQ ID NO: 155 - protein, 7 aa, Mus musculus
SASYRYS

SEQ ID NO: 156 - protein, 9 aa, Mus musculus
QQFYSYPYT

SEQ ID NO: 157 - protein, 123 aa, Mus musculus
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCAGGGIYYRYDRNYFDYWGQGTTLTVSS

SEQ ID NO: 158 - protein, 7 aa, Mus musculus
GYTFTDY

SEQ ID NO: 159 - protein, 6 aa, Mus musculus
NPNNGG

SEQ ID NO: 160 - protein, 14 aa, Mus musculus
GGIYYRYDRNYFDY

SEQ ID NO: 161 - protein, 107 aa, Mus musculus
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPFTFGSGTKLEIK

SEQ ID NO: 162 - protein, 11 aa, Mus musculus
SASQGISNYLN

SEQ ID NO: 163 - protein, 7 aa, Mus musculus
YTSSLHS

SEQ ID NO: 164 - protein, 9 aa, Artificial Sequence
  Description of Artificial Sequence Synthetic peptide
QQYSKLPFT

SEQ ID NO: 165 - protein, 117 aa, Mus musculus
EVKLVESEGGLVQPGSSMKLSCTASGFTFSDYYMAWVRQVPEKGLEWVANINYDGSNTSYLDSLKSRFIISRDNAKNILYLQMSSLKSEDTATYYCAREKFAAMDYWGQGTSVTVSS

SEQ ID NO: 166 - protein, 7 aa, Mus musculus
GFTFSDY

SEQ ID NO: 167 - protein, 6 aa, Mus musculus
NYDGSN

SEQ ID NO: 168 - protein, 8 aa, Mus musculus
EKFAAMDY

SEQ ID NO: 169 - protein, 108 aa, Mus musculus
DIVMTQSHKFMSTSFGDRVSITCKASQDVSNALAWYQQKPGHSPKLLIFSASYRYTGVPDRFTGSGSGTDFTFTISSVQAEDLAVYYCQQHYSTPWTFGGGTKLEIKR

SEQ ID NO: 170 - protein, 11 aa, Mus musculus
KASQDVSNALA

SEQ ID NO: 171 - protein, 7 aa, Artificial Sequence
  Description of Artificial Sequence Synthetic variable light chain CDR peptide
SASYRYT

SEQ ID NO: 172 - protein, 9 aa, Mus musculus
QQHYSTPWT

SEQ ID NO: 173 - protein, 121 aa, Artificial Sequence
  Description of Artificial Sequence Synthetic polypeptide
EVQLVESGGGLVQPGGSLRLSCAASGFTFTRHTIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 174 - protein, 10 aa, Artificial Sequence
  Description of Artificial Sequence Synthetic peptide
GFTFTRHTIH

SEQ ID NO: 175 - protein, 17 aa, Artificial Sequence
  Description of Artificial Sequence Synthetic peptide
RISPANGNTNYADSVKG

SEQ ID NO: 176 - protein, 12 aa, Artificial Sequence
  Description of Artificial Sequence Synthetic peptide
WIGSRELYIMDY

SEQ ID NO: 177 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYRIQPTFGQGTKVEIKR

SEQ ID NO: 178 - protein, 11 aa, Artificial Sequence
  Description of Artificial Sequence Synthetic peptide
RASQDVSTAVA

SEQ ID NO: 179 - protein, 7 aa, Artificial Sequence
  Description of Artificial Sequence Synthetic peptide
SASFLYS

SEQ ID NO: 180 - protein, 9 aa, Artificial Sequence
  Description of Artificial Sequence Synthetic peptide
QQSYRIQPT

SEQ ID NO: 181 - protein, 121 aa, Artificial Sequence
  Description of Artificial Sequence Synthetic polypeptide
EVQLVESGGGLVQPGGSLRLSCAASGFTFSSTAIHWVRQAPGKGLEWVARISPANGNTNYADSVKGRFTISADTSKNTAYLQMNSLRAEDTAVYYCARWIGSRELYIMDYWGQGTLVTVSS

SEQ ID NO: 182 - protein, 10 aa, Artificial Sequence
  Description of Artificial Sequence Synthetic peptide
GFTFSSTAIH

SEQ ID NO: 183 - protein, 17 aa, Artificial Sequence
  Description of Artificial Sequence Synthetic peptide
RISPANGNTNYADSVKG

SEQ ID NO: 184 - protein, 12 aa, Artificial Sequence
  Description of Artificial Sequence Synthetic peptide
WIGSRELYIMDY

SEQ ID NO: 185 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYPALHTFGQGTKVEIKR

SEQ ID NO: 186 - protein, 11 aa, Artificial Sequence
  Description of Artificial Sequence Synthetic peptide
RASQDVSTAVA

SEQ ID NO: 187 - protein, 7 aa, Artificial Sequence
  Description of Artificial Sequence Synthetic peptide
SASFLYS

SEQ ID NO: 188 - protein, 9 aa, Artificial Sequence
  Description of Artificial Sequence Synthetic peptide
QQSYPALHT

SEQ ID NO: 189 - protein, 125 aa, Artificial Sequence
  Description of Artificial Sequence Synthetic polypeptide
EVQLVESGGGLVKPGGSLRLSCAASGFPFSKLGMVWVRQAPGKGLEWVSTISSGGGYTYYPDSVKGRFTISRDNAKNSLYLQMNSLRAEDTAVYYCAREGISFQGGTYTYVMDYWGQGTLVTVSS

SEQ ID NO: 190 - protein, 10 aa, Artificial Sequence
  Description of Artificial Sequence Synthetic peptide
GFPFSKLGMV

SEQ ID NO: 191 - protein, 17 aa, Artificial Sequence
  Description of Artificial Sequence Synthetic peptide
TISSGGGYTYYPDSVKG

SEQ ID NO: 192 - protein, 16 aa, Artificial Sequence
  Description of Artificial Sequence Synthetic peptide
EGISFQGGTYTYVMDY

SEQ ID NO: 193 - protein, 112 aa, Artificial Sequence
  Description of Artificial Sequence Synthetic polypeptide
DIVMTQSPLSLPVTPGEPASISCRSSKSLLHRNGITYSYWYLQKPGQSPQLLIYQLSNLASGVPDRFSGSGSGTDFTLKISRVEAEDVGVYYCYQNLELPLTFGQGTKVEIK

SEQ ID NO: 194 - protein, 16 aa, Artificial Sequence
  Description of Artificial Sequence Synthetic peptide
RSSKSLLHRNGITYSY

SEQ ID NO: 195 - protein, 7 aa, Artificial Sequence
  Description of Artificial Sequence Synthetic peptide
QLSNLAS

SEQ ID NO: 196 - protein, 9 aa, Artificial Sequence
  Description of Artificial Sequence Synthetic peptide
YQNLELPLT

SEQ ID NO: 197 - DNA, 2076 bp, Homo sapiens
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaagaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtatggtcagcacactcggggcctacacggatggccacagccgtcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaaggggccgtgacagccgttgccatctgctgccggagccggcacctggcgcaggcctcccaggagctccag

SEQ ID NO: 198 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

(19) United States
(12) Patent Application Publication
(10) Pub. No.: US 2015/0231236 A1
(43) Pub. Date: Aug. 20, 2015

(54) METHODS FOR TREATING PATIENTS WITH HYPERCHOLESTEROLEMIA THAT IS NOT ADEQUATELY CONTROLLED BY MODERATE-DOSE STATIN THERAPY

(71) Applicants: Regeneron Pharmaceuticals, Inc., Tarrytown, NY (US); Sanofi, Paris (FR)

(72) Inventors: Robert C. Pordy, Ardsley, NY (US); William J. Sasiela, Bloomingdale, NJ (US); Joyce B. Harp, Montclair, NJ (US); Corinne Hanotin, Paris (FR); Laurence Bessac, Paris (FR)

(21) Appl. No.: 14/621,000

(22) Filed: Feb. 12, 2015

Related U.S. Application Data
(60) Provisional application No. 61/939,857, filed on Feb. 14, 2014.
(60) Provisional application No. 62/000,162, filed on May 19, 2014.
(60) Provisional application No. 62/025,094, filed on Jul. 16, 2014.
(60) Provisional application No. 62/052,227, filed on Sep. 18, 2014.

(30) Foreign Application Priority Data
Oct. 29, 2014 (EP) 14306729.6

(51) Int. Cl. A61K 39/395; A61K 31/505; A61K 31/40

(52) U.S. Cl. CPC A61K 39/3955; A61K 31/40; A61K 31/505; A61K 2039/505

(57) ABSTRACT

The present invention provides methods for treating hypercholesterolemia. The methods of the present invention comprise administering to a patient a pharmaceutical composition comprising a PCSK9 inhibitor. In certain embodiments, the PCSK9 inhibitor is an anti-PCSK9 antibody such as the exemplary antibody referred to herein as mAb316P. The methods of the present invention are useful for treating patients with hypercholesterolemia that is not adequately controlled by moderate-dose statin therapy.

### CROSS-REFERENCE TO RELATED APPLICATIONS

This application claims the benefit under 35 U.S.C. §119(e) of US provisional application Nos. 61/939,857, filed on Feb. 14, 2014; 62/000,162, filed on May 19, 2014; 62/025,094, filed on Jul. 16, 2014; and 62/052,227, filed on Sep. 18, 2014. This application also claims the benefit of priority to European Patent Application No. 14306729.6. The disclosures of the aforementioned patent applications are herein incorporated by reference in their entireties.

### FIELD OF THE INVENTION

The present invention relates to the field of therapeutic treatments of diseases and disorders which are associated with elevated levels of lipids and lipoproteins. More specifically, the invention relates to the use of PCSK9 inhibitors to treat patients with hypercholesterolemia that is not adequately controlled by moderate-dose statin therapy.

### BACKGROUND

Hypercholesterolemia, particularly an increase in low-density lipoprotein (LDL) cholesterol (LDL-C) levels, constitutes a major risk for the development of atherosclerosis and coronary heart disease (CHD) (Sharrett et al., 2001, Circulation 104:1108-1113). Low-density lipoprotein cholesterol is identified as the primary target of cholesterol lowering therapy and is accepted as a valid surrogate therapeutic endpoint. Numerous studies have demonstrated that reducing LDL-C levels reduces the risk of CHD with a strong direct relationship between LDL-C levels and CHD events; for each 1 mmol/L (˜40 mg/dL) reduction in LDL-C, cardiovascular disease (CVD) mortality and morbidity is lowered by 22%. Greater reductions in LDL-C produce greater reduction in events, and comparative data of intensive versus standard statin treatment suggest that the lower the LDL-C level, the greater the benefit in patients at very high cardiovascular (CV) risk.

Current LDL-C lowering medications include statins, cholesterol absorption inhibitors (e.g., ezetimibe [EZE]), fibrates, niacin, and bile acid sequestrants. Statins are the most commonly prescribed, as they have shown a greater ability to lower LDL-C and reduce CHD events. However, many patients at risk of cardiovascular disease (CVD) have poorly controlled low-density lipoprotein cholesterol (LDL-C) despite statin therapy.

### BRIEF SUMMARY OF THE INVENTION

The present invention provides methods for treating hypercholesterolemia. In particular, the methods of the present invention are useful for treating patients with hypercholesterolemia that is not adequately controlled by moderate-dose statin therapy.

According to one aspect, the methods of the present invention comprise administering one or more doses of a PCSK9 inhibitor to a patient with hypercholesterolemia that is not adequately controlled by moderate-dose statin therapy (i.e., hypercholesterolemia that is not adequately controlled by moderate-dose statin therapy in the absence of a PCSK9 inhibitor, with or without other lipid modifying therapy). According to certain embodiments of the present invention, the PCSK9 inhibitor is administered to the patient as an add-on therapy to the patient's existing statin therapy.

According to another aspect, the methods of the present invention comprise selecting a patient who is on a therapeutic regimen comprising a daily dose of a statin (e.g., a moderate-dose statin therapy), and administering to the patient one or more doses of a PCSK9 inhibitor in combination with (i.e., “on top of”) the statin therapy.

The present invention also provides pharmaceutical compositions comprising a PCSK9 inhibitor for use in treating a patient with hypercholesterolemia that is no controlled by moderate-dose statin therapy and a pharmaceutically acceptable carrier.

Other embodiments of the present invention will become apparent from a review of the ensuing detailed description.

### BRIEF DESCRIPTION OF THE FIGURES

FIG. 1 is an illustration of the study design for the clinical trial described in Example 2, wherein patients on moderate-dose atorvastatin (ATV) therapy (20 mg or 40 mg daily) were randomized into the treatment groups as shown.

FIG. 2 is an illustration of the study design for the clinical trial described in Example 3, wherein patients on moderate-dose rosuvastatin (RSV) therapy (10 mg or 20 mg daily) were randomized into the treatment groups as shown.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

### Hypercholesterolemia not Adequately Controlled by Moderate-Dose Statin Therapy

The present invention relates generally to methods and compositions for treating patients who have hypercholesterolemia that is not adequately controlled by statins, i.e., hypercholesterolemia not adequately controlled by a therapeutic regimen comprising a daily moderate-dose of a statin. As used herein, the expression “not adequately controlled,” in reference to hypercholesterolemia, means that the patient's serum low-density lipoprotein cholesterol (LDL-C) concentration, total cholesterol concentration, and/or triglyceride concentration is not reduced to a recognized, medically-acceptable level (taking into account the patient's relative risk of coronary heart disease) after at least 4 weeks on a therapeutic regimen comprising a stable daily dose of a statin. For example, “a patient with hypercholesterolemia that is not adequately controlled by a statin” includes patients with a serum LDL-C concentration of greater than about 70 mg/dL, 100 mg/dL, 130 mg/dL, 140 mg/dL, or more (depending on the patient's underlying risk of heart disease) after the patient has been on a stable daily statin regimen for at least 4 weeks.

According to certain embodiments, the patient who is treatable by the methods of the present invention has hypercholesterolemia (e.g., a serum LDL-C concentration of greater than or equal to 70 mg/dL, or a serum LDL-C concentration greater than or equal to 100 mg/dL) despite taking a stable daily dose of a statin (with or without other lipid modifying therapy) for at least 4 weeks, 5 weeks, 6 weeks, or more. In certain embodiments, the patient's hypercholesterolemia is inadequately controlled by a moderate-dose statin therapy (also referred to herein as “a daily moderate-dose therapeutic statin regimen”).

As used herein, “moderate-dose statin therapy” or “daily moderate-dose therapeutic statin regimen,” means a therapeutic regimen comprising the administration of daily dose of a statin that is below the maximally tolerated dose for a particular patient. (Maximally tolerated dose means the highest dose of statin that can be administered to a patient without causing unacceptable adverse side effects in the patient). A moderate dose of a statin may also be referred to herein as a “submaximal dose.” “Moderate-dose statin therapy” includes but is not limited to, e.g., 10 mg of atorvastatin daily, 20 mg of atorvastatin daily, 40 mg of atorvastatin daily, 5 mg of rosuvastatin daily, 10 mg of rosuvastatin daily, and 20 mg of rosuvastatin daily.

The present invention also includes methods for treating patients with hypercholesterolemia that is not adequately controlled by moderate-dose statin therapy comprising daily administration of other statins such as cerivastatin, pitavastatin, fluvastatin, lovastatin, and pravastatin.

### Patient Selection

The present invention includes methods and composition useful for treating patients who have hypercholesterolemia that is not adequately controlled by a daily moderate-dose therapeutic statin regimen. The patients who are treatable by the methods of the present invention may also exhibit one or more of additional selection criteria. For example, a patient may be selected for treatment with the methods of the present invention if the patient is diagnosed with or identified as being at risk of developing a hypercholesterolemia condition such as, e.g., heterozygous Familial Hypercholesterolemia (heFH), homozygous Familial Hypercholesterolemia (hoFH), Autosomal Dominant Hypercholesterolemia (ADH, e.g., ADH associated with one or more gain-of-function mutations in the PCSK9 gene), autosomal recessive hypercholesterolemia (ARH, e.g., ARH associated with mutations in LDLRAP1), as well as incidences of hypercholesterolemia that are distinct from Familial Hypercholesterolemia (nonFH).

According to certain embodiments, the patient may be selected on the basis of having a history of coronary heart disease (CHD). As used herein a “history of CHD” (or “documented history of CHD”) includes one or more of: (i) acute myocardial infarction (MI); (ii) silent MI; (iii) unstable angina; (iv) coronary revascularization procedure (e.g., percutaneous coronary intervention [PCI] or coronary artery bypass graft surgery [CABG]); and/or (v) clinically significant CHD diagnosed by invasive or non-invasive testing (such as coronary angiography, stress test using treadmill, stress echocardiography or nuclear imaging).

According to certain embodiments, the patient may be selected on the basis of having non-coronary heart disease cardiovascular disease (“non-CHD CVD”). As used herein, “non-CHD CVD” includes one or more of: (i) documented previous ischemic stroke with a focal ischemic neurological deficit that persisted more than 24 hours, considered as being of atherothrombotic origin; (ii) peripheral arterial disease; (iii) abdominal aortic aneurysm; (iv) atherosclerotic renal artery stenosis; and/or (v) carotid artery disease (transient ischemic attacks or >50% obstruction of a carotid artery).

According to certain embodiments, the patient may be selected on the basis of having one or more additional risk factors such as, e.g., (i) documented moderate chronic kidney disease (CKD) as defined by 30≦eGFR<60 mL/min/1.73 m2 for 3 months or more; (ii) type 1 or type 2 diabetes mellitus with or without target organ damage (e.g., retinopathy, nephropathy, microalbuminuria); (iii) a calculated 10-year fatal CVD risk SCORE ≧5% (ESC/EAS Guidelines for the management of dyslipidemias, Conroy et al., 2003, Eur. Heart J. 24:987-1003).

According to certain embodiments, the patient may be selected on the basis of having one or more additional risk factors selected from the group consisting of age (e.g., older than 40, 45, 50, 55, 60, 65, 70, 75, or 80 years), race, national origin, gender (male or female), exercise habits (e.g., regular exerciser, non-exerciser), other preexisting medical conditions (e.g., type-II diabetes, high blood pressure, etc.), and current medication status (e.g., currently taking beta blockers, niacin, ezetimibe, fibrates, omega-3 fatty acids, bile acid resins, etc.).

According to the present invention, patients may be selected on the basis of a combination of one or more of the foregoing selection criteria or therapeutic characteristics. For example, according to certain embodiments, a patient suitable for treatment with the methods of the present invention, in addition to having hypercholesterolemia that is not adequately controlled by a daily moderate-dose therapeutic statin regimen, may further be selected on the basis of having heFH or non-FH in combination with: (i) a history of documented CHD, (ii) non-CHD CVD, and/or (iii) diabetes mellitus with target organ damage; such patients may also be selected on the basis of having a serum LDL-C concentration of greater than or equal to 70 mg/dL.

According to certain other embodiments, a patient suitable for treatment with the methods of the present invention, in addition to having hypercholesterolemia that is not adequately controlled by a daily moderate-dose therapeutic statin regimen, may further be selected on the basis of having heFH or non-FH without CHD, or non-CHD CVD, but having either (i) a calculated 10-year fatal CVD risk SCORE ≧5%; or (ii) diabetes mellitus without target organ damage; such patients may also be selected on the basis of having a serum LDL-C concentration of greater than or equal to 100 mg/dL.

### Administration of a PCSK9 Inhibitor as Add-on Therapy to Moderate-Dose Statin Therapy

The present invention includes methods wherein a patient with hypercholesterolemia that is not adequately controlled by a stable daily moderate-dose therapeutic statin regimen in the absence of a PCSK9 inhibitor is administered a PCSK9 inhibitor according to a particular dosing amount and frequency, and wherein the PCSK9 inhibitor is administered as an add-on to the patient's therapeutic statin regimen. For example, according to certain embodiments, if a patient has hypercholesterolemia that is not adequately controlled despite being on a stable daily moderate-dose therapeutic statin regimen comprising, e.g., 20 mg of atorvastatin, the patient may be administered a PCSK9 inhibitor at a particular amount and dosing interval while the patient continues his or her stable daily therapeutic statin regimen (e.g., 20 mg of atorvastatin daily).

The methods of the present invention include add-on therapeutic regimens wherein the PCSK9 inhibitor is administered as add-on therapy to the same stable daily moderate-dose therapeutic statin regimen (i.e., same dosing amount of statin) that the patient was on prior to receiving the PCSK9 inhibitor. In other embodiments, the PCSK9 inhibitor is administered as add-on therapy to a daily moderate-dose therapeutic statin regimen comprising a statin in an amount that is more than or less than the dose of stain the patient was on prior to receiving the PCSK9 inhibitor. For example, after starting a therapeutic regimen comprising a PCSK9 inhibitor administered at a particular dosing frequency and amount, the daily dose of statin administered or prescribed to the patient may (a) stay the same, (b) increase, or (c) decrease (e.g., up-titrate or down-titrate) in comparison to the daily statin dose the patient was taking before starting the PCSK9 inhibitor therapeutic regimen, depending on the therapeutic needs of the patient.

### Therapeutic Efficacy

The methods of the present invention will result in the reduction in serum levels of one or more lipid component selected from the group consisting of LDL-C, ApoB100, non-HDL-C, total cholesterol, VLDL-C, triglycerides, Lp(a) and remnant cholesterol. For example, according to certain embodiments of the present invention, administration of a pharmaceutical composition comprising a PCSK9 inhibitor to a patient with hypercholesterolemia that is not adequately controlled by a stable daily moderate-dose therapeutic statin regimen, (e.g., administration of the PCSK9 inhibitor on top of the patient's moderate-dose statin therapy) will result in a mean percent reduction from baseline in serum low density lipoprotein cholesterol (LDL-C) of at least about 25%, 30%, 40%, 50%, 60%, or greater; a mean percent reduction from baseline in ApoB100 of at least about 25%, 30%, 40%, 50%, 60%, or greater; a mean percent reduction from baseline in non-HDL-C of at least about 25%, 30%, 40%, 50%, 60%, or greater; a mean percent reduction from baseline in total cholesterol of at least about 10%, 15%, 20%, 25%, 30%, 35%, or greater; a mean percent reduction from baseline in VLDL-C of at least about 5%, 10%, 15%, 20%, 25%, 30%, or greater; a mean percent reduction from baseline in triglycerides of at least about 5%, 10%, 15%, 20%, 25%, 30%, 35% or greater; and/or a mean percent reduction from baseline in Lp(a) of at least about 5%, 10%, 15%, 20%, 25%, or greater.

The present invention includes a method for treating a patient with hypercholesterolemia, the method comprising administering multiple doses of an anti-PCSK9 antibody to the patient at a dosing amount of about 75 to 150 mg per dose, and a dosing frequency of about once every two weeks, (or a dosing regimen in accordance with an up-titration dosing regimen as described elsewhere herein), wherein the patient exhibits hypercholesterolemia that is not adequately controlled by a moderate-dose statin therapy in the absence of the anti-PCSK9 antibody, wherein the moderate-dose statin therapy comprises a daily dose of about 20 mg of atorvastatin, and wherein, after about 24 weeks of treatment with the anti-PCSK9 antibody in combination with the moderate-dose statin therapy, the patient exhibits a reduction in LDL-C level from baseline of about 44%.

The present invention also includes a method for treating a patient with hypercholesterolemia, the method comprising administering multiple doses of an anti-PCSK9 antibody to the patient at a dosing amount of about 75 to 150 mg per dose, and a dosing frequency of about once every two weeks, (or a dosing regimen in accordance with an up-titration dosing regimen as described elsewhere herein), wherein the patient exhibits hypercholesterolemia that is not adequately controlled by a moderate-dose statin therapy in the absence of the anti-PCSK9 antibody, wherein the moderate-dose statin therapy comprises a daily dose of about 40 mg of atorvastatin, and wherein, after about 24 weeks of treatment with the anti-PCSK9 antibody in combination with the moderate-dose statin therapy, the patient exhibits a reduction in LDL-C level from baseline of about 54%.

The present invention also includes a method for treating a patient with hypercholesterolemia, the method comprising administering multiple doses of an anti-PCSK9 antibody to the patient at a dosing amount of about 75 to 150 mg per dose, and a dosing frequency of about once every two weeks, (or a dosing regimen in accordance with an up-titration dosing regimen as described elsewhere herein), wherein the patient exhibits hypercholesterolemia that is not adequately controlled by a moderate-dose statin therapy in the absence of the anti-PCSK9 antibody, wherein the moderate-dose statin therapy comprises a daily dose of about 10 mg of rosuvastatin, and wherein, after about 24 weeks of treatment with the anti-PCSK9 antibody in combination with the moderate-dose statin therapy, the patient exhibits a reduction in LDL-C level from baseline of about 51%.

The present invention also includes a method for treating a patient with hypercholesterolemia, the method comprising administering multiple doses of an anti-PCSK9 antibody to the patient at a dosing amount of about 75 to 150 mg per dose, and a dosing frequency of about once every two weeks, (or a dosing regimen in accordance with an up-titration dosing regimen as described elsewhere herein), wherein the patient exhibits hypercholesterolemia that is not adequately controlled by a moderate-dose statin therapy in the absence of the anti-PCSK9 antibody, wherein the moderate-dose statin therapy comprises a daily dose of about 20 mg of rosuvastatin, and wherein, after about 24 weeks of treatment with the anti-PCSK9 antibody in combination with the moderate-dose statin therapy, the patient exhibits a reduction in LDL-C level from baseline of about 36%.

### PCSK9 Inhibitors

The methods of the present invention comprise administering to a patient a therapeutic composition comprising a PCSK9 inhibitor. As used herein, a “PCSK9 inhibitor” is any agent which binds to or interacts with human PCSK9 and inhibits the normal biological function of PCSK9 in vitro or in vivo. Non-limiting examples of categories of PCSK9 inhibitors include small molecule PCSK9 antagonists, peptide-based PCSK9 antagonists (e.g., “peptibody” molecules), and antibodies or antigen-binding fragments of antibodies that specifically bind human PCSK9.

The term “human proprotein convertase subtilisin/kexin type 9” or “human PCSK9” or “hPCSK9”, as used herein, refers to PCSK9 having the nucleic acid sequence shown in SEQ ID NO:197 and the amino acid sequence of SEQ ID NO:198, or a biologically active fragment thereof.

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-PCSK9 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3, (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PCSK9, as used in the context of the present invention, includes antibodies that bind PCSK9 or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PCSK9, however, has cross-reactivity to other antigens, such as PCSK9 molecules from other (non-human) species.

The anti-PCSK9 antibodies useful for the methods of the present invention may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present invention.

The present invention also includes methods involving the use of anti-PCSK9 antibodies comprising variants of any of the HCVR, LCVR, and/or CDR amino acid sequences disclosed herein having one or more conservative substitutions. For example, the present invention includes the use of anti-PCSK9 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

According to certain embodiments, the anti-PCSK9 antibody used in the methods of the present invention is an antibody with pH-dependent binding characteristics. As used herein, the expression “pH-dependent binding” means that the antibody or antigen-binding fragment thereof exhibits “reduced binding to PCSK9 at acidic pH as compared to neutral pH” (for purposes of the present disclosure, both expressions may be used interchangeably). For the example, antibodies “with pH-dependent binding characteristics” include antibodies and antigen-binding fragments thereof that bind PCSK9 with higher affinity at neutral pH than at acidic pH. In certain embodiments, the antibodies and antigen-binding fragments of the present invention bind PCSK9 with at least 3, 5, 10, 15, 20, 25, 30, 35, 40, 45, 50, 55, 60, 65, 70, 75, 80, 85, 90, 95, 100, or more times higher affinity at neutral pH than at acidic pH.

According to this aspect of the invention, the anti-PCSK9 antibodies with pH-dependent binding characteristics may possess one or more amino acid variations relative to the parental anti-PCSK9 antibody. For example, an anti-PCSK9 antibody with pH-dependent binding characteristics may contain one or more histidine substitutions or insertions, e.g., in one or more CDRs of a parental anti-PCSK9 antibody. Thus, according to certain embodiments of the present invention, methods are provided comprising administering an anti-PCSK9 antibody which comprises CDR amino acid sequences (e.g., heavy and light chain CDRs) which are identical to the CDR amino acid sequences of a parental anti-PCSK9 antibody, except for the substitution of one or more amino acids of one or more CDRs of the parental antibody with a histidine residue. The anti-PCSK9 antibodies with pH-dependent binding may possess, e.g., 1, 2, 3, 4, 5, 6, 7, 8, 9, or more histidine substitutions, either within a single CDR of a parental antibody or distributed throughout multiple (e.g., 2, 3, 4, 5, or 6) CDRs of a parental anti-PCSK9 antibody. For example, the present invention includes the use of anti-PCSK9 antibodies with pH-dependent binding comprising one or more histidine substitutions in HCDR1, one or more histidine substitutions in HCDR2, one or more histidine substitutions in HCDR3, one or more histidine substitutions in LCDR1, one or more histidine substitutions in LCDR2, and/or one or more histidine substitutions in LCDR3, of a parental anti-PCSK9 antibody.

As used herein, the expression “acidic pH” means a pH of 6.0 or less (e.g., less than about 6.0, less than about 5.5, less than about 5.0, etc.). The expression “acidic pH” includes pH values of about 6.0, 5.95, 5.90, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

Non-limiting examples of anti-PCSK9 antibodies that can be used in the context of the present invention include, e.g., alirocumab, evolocumab, bococizumab, or antigen-binding portions thereof.

### Preparation of Human Antibodies

Methods for generating human antibodies in transgenic mice are known in the art. Any such known methods can be used in the context of the present invention to make human antibodies that specifically bind to human PCSK9.

Using VELOCIMMUNE™ technology (see, for example, U.S. Pat. No. 6,596,541, Regeneron Pharmaceuticals) or any other known method for generating monoclonal antibodies, high affinity chimeric antibodies to PCSK9 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies that can be used in the methods of the present invention possess high affinities, as described above, when measured by binding to antigen either immobilized on solid phase or in solution phase. The mouse constant regions are replaced with desired human constant regions to generate the fully human antibodies of the invention. While the constant region selected may vary according to specific use, high affinity antigen-binding and target specificity characteristics reside in the variable region.

Specific examples of human antibodies or antigen-binding fragments of antibodies that specifically bind PCSK9 which can be used in the context of the methods of the present invention include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs:1 and 11, or a substantially similar sequence thereof having at least 90%, at least 95%, at least 98% or at least 99% sequence identity. Alternatively, specific examples of human antibodies or antigen-binding fragments of antibodies that specifically bind PCSK9 which can be used in the context of the methods of the present invention include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs 37, 45, 53, 61, 69, 77, 85, 93, 101, 109, 117, 125, 133, 141, 149, 157, 165, 173, 181, and 189, or a substantially similar sequence thereof having at least 90%, at least 95%, at least 98% or at least 99% sequence identity. The antibody or antigen-binding fragment may comprise the three light chain CDRs (LCVR1, LCVR2, LCVR3) contained within a light chain variable region (LCVR) having an amino acid sequence selected from the group consisting of SEQ ID NOs 6 and 15, or a substantially similar sequence thereof having at least 90%, at least 95%, at least 98% or at least 99% sequence identity. Alternatively, the antibody or antigen-binding fragment may comprise the three light chain CDRs (LCVR1, LCVR2, LCVR3) contained within a light chain variable region (LCVR) having an amino acid sequence selected from the group consisting of SEQ ID NOs 41, 49, 57, 65, 73, 81, 89, 97, 105, 113, 121, 129, 137, 145, 153, 161, 169, 177, 185, and 193, or a substantially similar sequence thereof having at least 90%, at least 95%, at least 98% or at least 99% sequence identity.

Sequence identity between two amino acids sequences is determined over the entire length of the reference amino acid sequence, i.e. the amino acid sequence identified with a SEQ ID NO, using the best sequence alignment and/or over the region of the best sequence alignment between the two amino acid sequences, wherein the best sequence alignment can be obtained with art known tools, e.g. Align, using standard settings, preferably EMBOSS::needle, Matrix: Blosum62, Gap Open 10.0, Gap Extend 0.5.

In certain embodiments of the present invention, the antibody or antigen-binding protein comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs:1/6 and 11/15. Alternatively, in certain embodiments of the present invention, the antibody or antigen-binding protein comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs:37/41, 45/49, 53/57, 61/65, 69/73, 77/81, 85/89, 93/97, 101/105, 109/113, 117/121, 125/129, 133/137, 141/145, 149/153, 157/161, 165/169, 173/177, 181/185, and 189/193.

In certain embodiments of the present invention, the anti-PCSK9 antibody, or antigen-binding protein, that can be used in the methods of the present invention has HCDR1/HCDR2/HCDR3/LCDR1/LCDR2/LCDR3 amino acid sequences selected from SEQ ID NOs: 2/3/4/7/8/10 (mAb316P [also referred to as “REGN727,” or “alirocumab”]) and 12/13/14/16/17/18 (mAb300N) (See US Patent App. Publ No. 2010/0166768) and 12/13/14/16/17/18, wherein SEQ ID NO:16 comprises a substitution of histidine for leucine at amino acid residue 30 (L30H).

In certain embodiments of the present invention, the antibody or antigen-binding protein comprises HCVR/LCVR amino acid sequence pairs selected from the group consisting of SEQ ID NOs:1/6 and 11/15. In certain exemplary embodiments, the antibody or antigen-binding protein comprises an HCVR amino acid sequence of SEQ ID NO:1 and an LCVR amino acid sequence of SEQ ID NO:6. In certain exemplary embodiments, the antibody or antigen-binding protein comprises an HCVR amino acid sequence of SEQ ID NO:11 and an LCVR amino acid sequence of SEQ ID NO:15. In certain exemplary embodiments, the antibody or antigen-binding protein comprises an HCVR amino acid sequence of SEQ ID NO:11 and an LCVR amino acid sequence of SEQ ID NO:15 comprising a substitution of histidine for leucine at amino acid residue 30 (L30H).

### Pharmaceutical Compositions and Methods of Administration

The present invention includes methods which comprise administering a PCSK9 inhibitor to a patient, wherein the PCSK9 inhibitor is contained within a pharmaceutical composition. The pharmaceutical compositions of the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of PCSK9 inhibitor (e.g., anti-PCSK9 antibody) administered to a subject according to the methods of the present invention is, generally, a therapeutically effective amount. As used herein, the phrase “therapeutically effective amount” means a dose of PCSK9 inhibitor that results in a detectable reduction (at least about 5%, 10%, 15%, 20%, 25%, 30%, 35%, 40%, 45%, 50%, 55%, 60%, 65%, 70%, 75%, or more from baseline) in one or more parameters selected from the group consisting of LDL-C, ApoB100, non-HDL-C, total cholesterol, VLDL-C, triglycerides, Lp(a) and remnant cholesterol.

In the case of an anti-PCSK9 antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-PCSK9 antibody.

The amount of anti-PCSK9 antibody contained within the individual doses may be expressed in terms of milligrams of antibody per kilogram of patient body weight (i.e., mg/kg). For example, the anti-PCSK9 antibody may be administered to a patient at a dose of about 0.0001 to about 10 mg/kg of patient body weight.

### Combination Therapies

As described elsewhere herein, the methods of the present invention may comprise administering a PCSK9 inhibitor to a patient in combination with the patient's previously prescribed stable daily moderate-dose therapeutic statin regimen. According to certain embodiments of the present invention, additional therapeutic agents, besides a statin, may be administered to the patient in combination with the PCSK9 inhibitor. Examples of such additional therapeutic agents include e.g., (1) an agent which inhibits cholesterol uptake and or bile acid re-absorption (e.g., ezetimibe); (2) an agent which increase lipoprotein catabolism (such as niacin); and/or (3) activators of the LXR transcription factor that plays a role in cholesterol elimination such as 22-hydroxycholesterol.

### Administration Regimens

According to certain embodiments of the present invention, multiple doses of a PCSK9 inhibitor (i.e., a pharmaceutical composition comprising a PCSK9 inhibitor) may be administered to a subject over a defined time course (e.g., on top of a daily therapeutic statin regimen). The methods according to this aspect of the invention comprise sequentially administering to a subject multiple doses of a PCSK9 inhibitor. As used herein, “sequentially administering” means that each dose of PCSK9 inhibitor is administered to the subject at a different point in time, e.g., on different days separated by a predetermined interval (e.g., hours, days, weeks or months). The present invention includes methods which comprise sequentially administering to the patient a single initial dose of a PCSK9 inhibitor, followed by one or more secondary doses of the PCSK9 inhibitor, and optionally followed by one or more tertiary doses of the PCSK9 inhibitor.

The terms “initial dose,” “secondary doses,” and “tertiary doses,” refer to the temporal sequence of administration of the individual doses of a pharmaceutical composition comprising a PCSK9 inhibitor. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the PCSK9 inhibitor, but generally may differ from one another in terms of frequency of administration. In certain embodiments, however, the amount of PCSK9 inhibitor contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

According to exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 261/2, or more) weeks after the immediately preceding dose. The phrase “the immediately preceding dose,” as used herein, means, in a sequence of multiple administrations, the dose of antigen-binding molecule which is administered to a patient prior to the administration of the very next dose in the sequence with no intervening doses.

The methods according to this aspect of the invention may comprise administering to a patient any number of secondary and/or tertiary doses of a PCSK9 inhibitor. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2, 4, 6, 8 or more weeks after the immediately preceding dose. Similarly, in embodiments involving multiple tertiary doses, each tertiary dose may be administered at the same frequency as the other tertiary doses. For example, each tertiary dose may be administered to the patient 1 to 2, 4, 6, 8 or more weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

The present invention includes administration regimens comprising an up-titration option (also referred to herein as “dose modification”). As used herein, an “up-titration option” means that, after receiving a particular number of doses of a PCSK9 inhibitor, if a patient has not achieved a specified reduction in one or more defined therapeutic parameters, the dose of the PCSK9 inhibitor is thereafter increased. For example, in the case of a therapeutic regimen comprising administration of 75 mg doses of an anti-PCSK9 antibody to a patient at a frequency of once every two weeks, if after 8 weeks (i.e., 5 doses administered at Week 0, Week 2 and Week 4, Week 6 and Week 8), the patient has not achieved a serum LDL-C concentration of less than 70 mg/dL, then the dose of anti-PCSK9 antibody is increased to e.g., 150 mg administered once every two weeks thereafter (e.g., starting at Week 10 or Week 12, or later).

In certain embodiments, the anti-PCSK9 antibody is administered to a subject at a dose of about 75 mg every two weeks, for example for at least three doses.

In certain embodiments, the anti-PCSK9 antibody is administered to a subject at a dose of about 150 mg every two weeks, for example for at least three doses.

In some embodiments, the antibody is administered to a subject at a dose of about 75 mg every two weeks for 12 weeks, and the dose remains at 75 mg every two weeks if, at week 8, the subject's LDL-C value was less than 100 mg/dl and a 30% reduction of LDL-C.

In other embodiments, the antibody is administered to a subject at a dose of about 75 mg every two weeks for 12 weeks, and the dose is titrated up to about 150 mg every two weeks if, at week 8, the subject's LDL-C value was greater than or equal to 100 mg/dl.

In some embodiments, the antibody is administered to a subject at a dose of about 75 mg every two weeks for 12 weeks, and the dose remains at 75 mg every two weeks if, at week 8, the subject's LDL-C value was less than 70 mg/dl and a 30% reduction of LDL-C.

In another embodiment, the antibody is administered to a subject at a dose of about 300 mg every four weeks.

In a further embodiment, the antibody is administered to a subject at a dose of about 300 mg every four weeks for a total of three doses, and the dose is changed to 150 mg every two weeks for another 36 weeks if, at week 8, the subject did not achieve a pre-determined treatment goal or the subject did not have at least a 30% reduction of LDL-C from baseline.

In certain embodiments, the anti-PCSK9 antibody is administered to a subject at a dose of about 150 mg every four weeks for at least three doses.

In some embodiments, the antibody is administered to a subject at a dose of about 150 mg every four weeks for 12 weeks, and the dose remains at 150 mg every four weeks if, at week 8, the subject's LDL-C value was less than 100 mg/dl and a 30% reduction of LDL-C.

In other embodiments, the antibody is administered to a subject at a dose of about 150 mg every four weeks for 12 weeks, and the dose is titrated up to about 300 mg every two weeks if, at week 8, the subject's LDL-C value was greater than or equal to 100 mg/dl.

In some embodiments, the antibody is administered to a subject at a dose of about 150 mg every four weeks for 12 weeks, and the dose remains at 150 mg every four weeks for another 12 weeks if, at week 8, the subject's LDL-C value was less than 70 mg/dl and a 30% reduction of LDL-C.

In another embodiment, the antibody is administered to a subject at a dose of about 300 mg every four weeks.

In a further embodiment, the antibody is administered to a subject at a dose of about 300 mg every four weeks for a total of three doses, and the dose is changed to 150 mg every two weeks for another 36 weeks if, at week 8, the subject did not achieve a pre-determined treatment goal or the subject did not have at least a 30% reduction of LDL-C from baseline.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Generation of Human Antibodies to Human PCSK9

Human anti-PCSK9 antibodies were generated as described in U.S. Pat. No. 8,062,640. The exemplary PCSK9 inhibitor used in the following Example is the human anti-PCSK9 antibody designated “mAb316P,” also known as “REGN727,” or “alirocumab.” mAb316P has the following amino acid sequence characteristics: a heavy chain comprising SEQ ID NO:5 and a light chain comprising SEQ ID NO:9; a heavy chain variable region (HCVR) comprising SEQ ID NO:1 and a light chain variable domain (LCVR) comprising SEQ ID NO:6; a heavy chain complementarity determining region 1 (HCDR1) comprising SEQ ID NO:2, a HCDR2 comprising SEQ ID NO:3, a HCDR3 comprising SEQ ID NO:4, a light chain complementarity determining region 1 (LCDR1) comprising SEQ ID NO:7, a LCDR2 comprising SEQ ID NO:8 and a LCDR3 comprising SEQ ID NO:10.

### Example 2

### A Randomized, Double-Blind Study of the Efficacy and Safety of an Anti-PCSK9 Antibody (“mAb316P”) Added-on to Atorvastatin Versus Ezetimibe Added-on to Atorvastatin Versus Atorvastatin Increase Versus Switch to Rosuvastatin in Patients Who are not Controlled on Atorvastatin

### Introduction

The objective of the present study was to compare mAb316P as add-on therapy to submaximal doses (i.e., “moderate doses”) of atorvastatin in comparison with ezetimibe (EZE) as add-on therapy to submaximal doses of atorvastatin, in comparison with doubling the atorvastatin dose, or in comparison with switching from atorvastatin to rosuvastatin, in patients at high cardiovascular (CV) risk who have failed to reach their LDL-C treatment goal and require additional pharmacological management, with the exception of EZE, which is an active comparator in the study. The definition of high CV risk in this study is based on existing guidelines (ESC/EAS Guidelines for the management of dyslipidaemias, Executive summary of the Third Report of the National Cholesterol Education Program 2001).

Maximizing the dose of atorvastatin is also a treatment option for patients who have failed to reach their LDL-C treatment goal (ESC/EAS Guidelines for the management of dyslipidaemia). Switching from atorvastatin to rosuvastatin is also done clinically because rosuvastatin has slightly greater LDL-C lowering potential. (Nicholls et al., 2011, N. Engl. J. Med. 365(22):2078-2087). Ezetimibe was selected as a comparator arm because it has been recommended as a treatment option for use in combination with statin.

The present study was double-blind and each patient received an injection Q2W and 2 oral capsules daily to maintain the double-blind.

### Study Objectives

The primary objective of this study was to evaluate the reduction of LDL-C by mAb316P as add-on therapy to atorvastatin in comparison with EZE as add-on therapy to atorvastatin, in comparison with doubling the atorvastatin dose, or in comparison with a therapy switch from atorvastatin to rosuvastatin, after 24 weeks of treatment in patients with hypercholesterolemia at high CV risk. The secondary objectives of this study were: (a) to evaluate the reduction of LDL-C by mAb316P 75 mg as add-on therapy to atorvastatin in comparison with EZE as add-on therapy to atorvastatin, in comparison with doubling of the atorvastatin dose, or in comparison with a switch from atorvastatin to rosuvastatin after 12 weeks of treatment; (b) to evaluate the effect of mAb316P on other lipid parameters (e.g., ApoB, non-HDL-C, total-C, Lp(a), HDL-C, TG levels, ApoA-1, etc.); (c) to evaluate the safety and tolerability of mAb316P; and (d) to evaluate the development of anti-mAb316P antibodies.

### Study Design

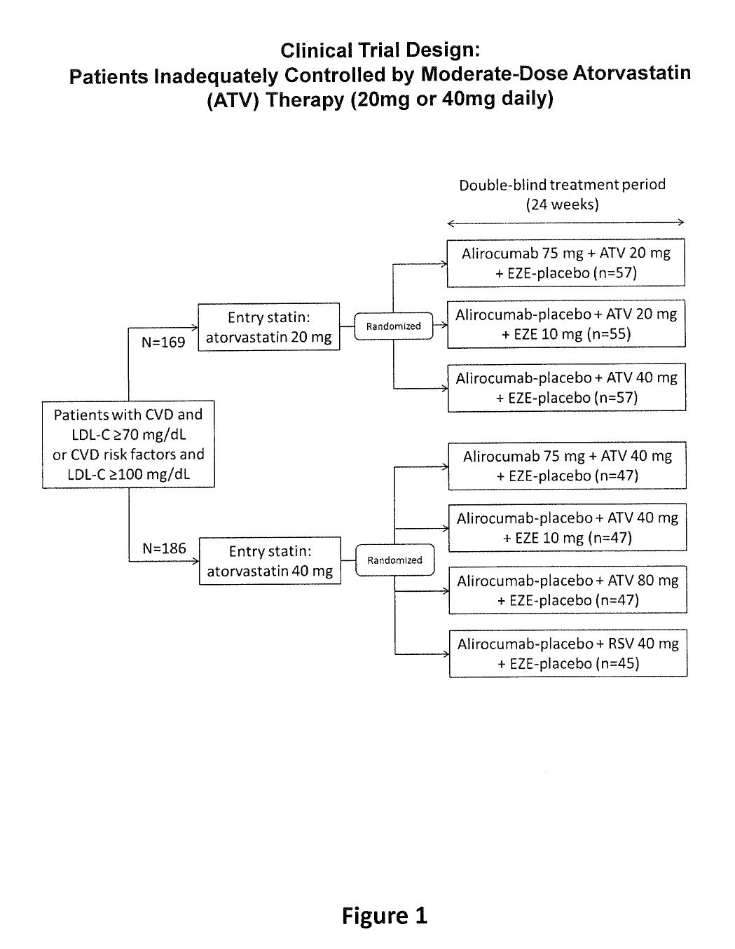

The present study was a randomized, double-blind, active-comparator, parallel-group study in patients at high CV risk with non-FH or heFH who are not adequately controlled with atorvastatin (20 mg or 40 mg) with or without other lipid-modifying therapy (LMT) (excluding EZE). The study design is illustrated in FIG. 1. Patients who entered the study were taking either atorvastatin 20 mg or atorvastatin 40 mg. The former patients were randomized to 1 of 3 treatment arms (arms 1 to 3); the latter patients were randomized to 1 of 4 treatment arms (arms 4 to 7). The treatment arms are as follows:

Patients on a 20 mg Atorvastatin Regimen:

(1) mAb316P+atorvastatin 20 mg+placebo-EZE; (2) Placebo-mAb316P+atorvastatin 40 mg+placebo-EZE; and (3) Placebo-mAb316P+atorvastatin 20 mg+EZE 10 mg.

Patients on a 40 mg Atorvastatin Regimen:

(4) mAb316P+atorvastatin 40 mg+placebo-EZE; (5) Placebo-mAb316P+atorvastatin 80 mg+placebo-EZE; (6). Placebo-mAb316P+rosuvastatin 40 mg+placebo-EZE; and (7) Placebo-mAb316P+atorvastatin 40 mg+EZE 10 mg.

Within each atorvastatin regimen, randomization will be stratified according to whether the patient has a prior history of either myocardial infarction (MI) or ischemic stroke (yes/no).

The present study consisted of:

(A) a screening period of up to 2 weeks, including an intermediate visit during which the patient or caregiver will be trained to self-inject/inject using a dose of placebo mAb316P;

(B) a double-blind treatment period of 24 weeks. Each patient received a SC injection Q2W (mAb316P or placebo-mAb316P) and was administered (or instructed to take) 2 oral blinded medications daily (a statin [atorvastatin or rosuvastatin] and EZE or placebo-EZE). The first injection of mAb316P or placebo-mAb316P was administered at the clinical site on day 1, after study assessments were completed, and as soon as possible after the patient was randomized into the study. The patient/caregiver administered subsequent injections outside of the clinic according to the dosing schedule. On days where the clinic study visit coincided with dosing, the dose of study drugs (injectable and oral) were administered after all study assessments had been performed and all laboratory samples collected. The last dose of mAb316P or placebo-mAb316P was administered at week 22. The last dose of daily oral study drugs was administered at week 24. At week 12, based on their baseline CV risk, certain patients randomized to mAb316P in a blinded manner, had their dose increased as follows:

(1) Patients with heFH or non-FH and a history of documented CHD, or non-CHD CVD, or diabetes mellitus with target organ damage: (a) Continue mAb316P 75 mg Q2W, if the week 8 LDL-C is <70 mg/dL (1.8 mmol/L), or (b) Dose up-titrate to mAb316P 150 mg Q2W, if the week 8 LDL-C is ≧70 mg/dL (1.8 mmol/L);

(2) Patients with heFH or non-FH, without CHD or non-CHD CVD, but with a calculated 10-year fatal CVD risk SCORE ≧5%, or with moderate CKD, or with diabetes mellitus but no target organ damage: (a) Continue mAb316P 75 mg Q2W, if the week 8 LDL-C is <100 mg/dL (2.59 mmol/L), or (b) Dose up-titrate to mAb316P 150 mg Q2W, if the week 8 LDL-C is ≧100 mg/dL (2.59 mmol/L). Lipid results will be blinded from specimens obtained after randomization (including week 8). The continuation of the 75 mg dose or dose up-titration to the 150 mg dose was done using an automated process without site or patient awareness.

(C) A follow-up period of 8 weeks.

Patients were asked to follow a stable diet (the National Cholesterol Education Program Adult Treatment Panel III Therapeutic Lifestyle Changes [NCEP ATP III-TLC] diet or equivalent diet) from screening to the end of study visit. The addition of other LMT was not permitted during the double-blind treatment period except under certain conditions.

### Patient Selection

The study population consisted of patients with hypercholesterolemia and established CHD or non-CHD CVD (defined below), or who were at high risk for CVD due other factors and who were not adequately controlled with a 20 mg or 40 mg daily dose of atorvastatin, with or without other LMT, except EZE.

Inclusion Criteria:

The patients enrolled in this study met conditions 1a or 1b (below) to be eligible for inclusion in the study:

1a. Patients with screening (visit 1) LDL-C≧70 mg/dL (1.81 mmol/L) who were not adequately controlled with a 20 mg or 40 mg stable daily dose of atorvastatin for at least 4 weeks before the screening visit (visit 1), with or without other LMT (excluding EZE). Patients with heFH or non-FH must also have had a history of documented CHD (defined below), or non-CHD CVD (defined below), or diabetes mellitus with target organ damage; OR

1 b. Patients with screening (visit 1) LDL-C≧100 mg/dL (2.59 mmol/L) who were not adequately controlled with a 20 mg or 40 mg daily dose of atorvastatin for at least 4 weeks before the screening visit (visit 1), with or without other LMT (excluding EZE). Patients must also have had heFH, or have non-FH, without CHD or non-CHD CVD, but with a calculated 10-year fatal CVD risk SCORE ≧5%, or with moderate CKD, or with diabetes mellitus but no target organ damage.

Note: Diagnosis of heFH is made either by genotyping or by clinical criteria.

Definitions for CHD, Non-CHD CVD, and Other Risk Factors:

A. A documented history of CHD (includes 1 or more of the following): i. Acute MI; ii. Silent MI; iii. Unstable angina; iv. Coronary revascularization procedure (e.g., percutaneous coronary intervention [PCI] or coronary artery bypass graft surgery [CABG]); and/or v. Clinically significant CHD diagnosed by invasive or non-invasive testing (such as coronary angiography, stress test using treadmill, stress echocardiography or nuclear imaging).

B. Non-CHD CVD (includes 1 or more of the following criteria): i. Documented previous ischemic stroke with a focal ischemic neurological deficit that persisted more than 24 hours, considered as being of atherothrombotic origin. CT or MRI is performed to rule out hemorrhage and non-ischemic neurological disease; ii. Peripheral arterial disease; iii. Abdominal aortic aneurysm; iv. Atherosclerotic renal artery stenosis; and/or v. Carotid artery disease (transient ischemic attacks or >50% obstruction of a carotid artery)

C. Other Risk Factors: i. Documented moderate CKD as defined by 30≦eGFR<60 mL/min/1.73 m2 for 3 months or more, including the screening visit; ii. Type 1 or type 2 diabetes mellitus with or without target organ damage (i.e., retinopathy, nephropathy, microalbuminuria); iii. A calculated 10-year fatal CVD risk SCORE ≧5% (ESC/EAS Guidelines for the management of dyslipidemias, Conroy 2003).

Exclusion Criteria:

Prospective patients who met any of the following criteria were excluded from the study:

1. LDL-C<70 mg/dL (<1.81 mmol/L) at the screening visit (week −2) in patients with history of documented CHD or non-CHD CVD.

2. LDL-C<100 mg/dL (<2.59 mmol/L) at the screening visit (week −2) in patients without history of documented CHD or non-CHD CVD, but with other risk factors.

3. Homozygous FH (clinically or previous genotyping).

4. Currently taking a statin that is not atorvastatin taken daily at 20 mg or 40 mg.

5. Currently taking EZE or had received EZE within 4 weeks of screening visit 1 (week −2).

6. Not on a stable dose of allowable LMT (excluding EZE) for at least 4 weeks and/or fenofibrate for at least 6 weeks prior to the screening visit (week −2) or from screening to randomization, as applicable.

7. Use of fibrates, other than fenofibrate within 6 weeks of the screening visit (week −2) or between screening and randomization visits.

8. Use of nutraceutical products or over-the-counter therapies that may affect lipids and which the dose amount has not been stable for at least 4 weeks prior to the screening visit (week −2), or between screening and randomization visits.

9. Use of red yeast rice products within 4 weeks of the screening visit (week −2) or between screening and randomization visits.

10. Patient who has received plasmapheresis treatment within 2 months prior to the screening visit (week −2), or has plans to receive it during the study.

11. Recent (within 3 months prior to the screening visit [week −2]) MI, unstable angina leading to hospitalization, PCI, CABG, uncontrolled cardiac arrhythmia, stroke, transient ischemic attack, carotid revascularization, endovascular procedure or surgical intervention for peripheral vascular disease.

12. Planned to undergo scheduled PCI, CABG, carotid or peripheral revascularization during the study.

13. Systolic blood pressure >160 mm Hg or diastolic blood pressure >100 mm Hg at screening visit and/or randomization visit.

14. History of New York Heart Association (NYHA) Class III or IV heart failure within the past 12 months.

15. Known history of hemorrhagic stroke.

16. Age <18 years or legal age of majority at the screening visit (week −2), whichever is greater.

17. Patients not previously instructed on a cholesterol-lowering diet prior to the screening visit (week-2).

18. Newly diagnosed (within 3 months prior to randomization visit [week 0]) or poorly controlled (hemoglobin A1c [HbA1c]>8.5%) diabetes.

19. Presence of any clinically significant uncontrolled endocrine disease known to influence serum lipids or lipoproteins. Note: patients on thyroid replacement therapy can be included if the dosage of thyroxine has been stable for at least 12 weeks prior to screening and the thyroid-stimulating hormone (TSH) level is within the normal range of the central laboratory at the screening visit.

20. History of bariatric surgery within 12 months prior to the screening visit (week −2).

21. Unstable weight defined by a variation >5 kg within 2 months prior to the screening visit (week −2).

22. Known history of loss of function of PCSK9 (i.e., genetic mutation or sequence variation).

23. Use of systemic corticosteroids, unless used as replacement therapy for pituitary/adrenal disease with a stable regimen for at least 6 weeks prior to randomization. Note: topical, intra-articular, nasal, inhaled and ophthalmic steroid therapies are not considered as “systemic” and are allowed.

24. Use of continuous estrogen or testosterone hormone replacement therapy unless the regimen has been stable in the past 6 weeks prior to the screening visit (week −2) and no plans to change the regimen during the study.

25. History of cancer within the past 5 years, except for adequately treated basal cell skin cancer, squamous cell skin cancer, or in situ cervical cancer.

26. Known history of HIV positive.

27. Patient who has taken any active investigational drugs within 1 month or 5 half-lives, whichever is longer.

28. Patient who has previously participated in any clinical trial of mAb316P or any other anti-PCSK9 monoclonal antibody.

29. Conditions/situations such as: (A) Any clinically significant abnormality identified at the time of screening that in the judgment of the Investigator or any sub-investigator would preclude safe completion of the study or constrain endpoints assessment such as major systemic diseases, patients with short life expectancy; or (B) Patients considered by the investigator or any sub-investigator as inappropriate for this study for any reason, e.g.: (i) Those deemed unable to meet specific protocol requirements, such as scheduled visits; (ii) Those deemed unable to administer or tolerate long-term injections as per the patient or the investigator; (iii) Investigator or any sub-investigator, pharmacist, study coordinator, other study staff or relative thereof directly involved in the conduct of the protocol, etc.; (iv) Presence of any other conditions (e.g., geographic, social, etc.) actual or anticipated, that the investigator feels would restrict or limit the patient's participation for the duration of the study.

30. Laboratory findings obtained at during screening period (not including randomization [week 0] labs, unless otherwise noted): (1) Positive test for hepatitis B surface antigen and/or hepatitis C antibody (confirmed by reflexive testing). (2) LDL-C>250 mg/dL (>6.47 mmol/L). (3) TG>400 mg/dL (>4.52 mmol/L) (1 repeat lab is allowed). (4) Positive serum or urine pregnancy test (including week 0) in women of childbearing potential. (5) eGFR<30 mL/min/1.73 m2 according to the 4-variable MDRD Study Equation (calculated by central laboratory) (6) Alanine aminotransferase (ALT) or aspartate aminotransferase (AST)>3× upper limit of normal (ULN) (1 repeat lab is allowed). (7) CPK>3×ULN (1 repeat lab is allowed). (8) TSH<lower limit of normal (LLN) or >ULN.

31. All contraindications to the active comparators (EZE, atorvastatin, rosuvastatin) and background therapies or warnings/precautions of use (when appropriate) as displayed in the respective National Product Labeling.

32. Known hypersensitivity to monoclonal antibody therapeutics.

33. Pregnant or breast-feeding women.

34. Women of childbearing potential with no effective contraceptive method of birth control and/or who are unwilling or unable to be tested for pregnancy.

### Study Treatments

Each patient received a subcutaneous (SC) injection once every two weeks (Q2W) (mAb316P or placebo-mAb316P) and took 2 oral blinded medications daily (a statin [atorvastatin or rosuvastatin] and EZE or placebo-EZE).

The injectable study treatment was a single SC injection of 1 mL for a 75 mg or 150 mg dose of mAb316P or placebo-mAb316P provided in an auto-injector, administered in the abdomen, thigh, or outer area of the upper arm. The first injection of study drug was administered at the clinical site, as soon as possible after the patient was randomized into the study. The patient was monitored at the clinical site for at least 30 minutes following the first injection. The patient/caregiver administered subsequent injections outside of the clinic, according to the dosing schedule. On days where the clinic study visit coincided with dosing, the dose of study drug was administered after all study assessments have been performed and all laboratory samples collected.

Subcutaneous dosing of study drug was administered Q2W at approximately the same time of day (based upon patient preference); it was acceptable for dosing to fall within a window of +/−3 days.

In the event an injection was delayed by more than 7 days or completely missed, the patient was instructed to return to the original schedule of study drug dosing without administering additional injections. If the delay was less than or equal to 7 days from the missed date the patient was instructed to administer the delayed injection and then resume the original dosing schedule.

Oral study treatments were a statin (atorvastatin or rosuvastatin) and EZE or placebo-EZE. The first dose of oral study drug was administered at the clinical site, as soon as possible after the patient was randomized into the study. The patients continued daily dosing with oral medication through week 22 (day 155). On days where the clinic study visit coincided with dosing, the dose of oral study drug was administered after all study assessments had been performed and all laboratory samples collected.

### Investigational Treatment

Sterile mAb316P drug product was supplied at a concentration of 75 mg/mL or 150 mg/mL in histidine, pH 6.0, polysorbate 20, and sucrose in an auto-injector.

Placebo matching mAb316P was supplied in the same formulation as mAb316P, without the addition of protein, in an auto-injector.

Ezetimibe 10 mg was provided as over-encapsulated tablets. Matching placebo capsules for EZE were supplied.

Atorvastatin 20 mg, 40 mg, and 80 mg, and rosuvastatin 40 mg were supplied as matching over-encapsulated tablets.

### Dose Modification (“Up-Titration Option”)

The dose of mAb316P was increased, in a blinded manner, from 75 mg to 150 mg SC Q2W, starting at week 12, based on baseline CV risk in the following circumstances:

A. Patients with heFH or non-FH and a history of documented CHD (defined elsewhere herein), or non-CHD CVD (defined elsewhere herein), or diabetes mellitus with target organ damage: (1) Continue mAb316P 75 mg Q2W, if the week 8 LDL-C is <70 mg/dL (1.8 mmol/L), or (2) Dose up-titrate to mAb316P 150 mg Q2W, if the week 8 LDL-C is ≧70 mg/dL (1.8 mmol/L).

B. Patients with heFH or non-FH, without CHD or non-CHD CVD (defined elsewhere herein), but with a calculated 10-year fatal CVD risk SCORE ≧5%, or with moderate CKD, or with diabetes mellitus but no target organ damage: (1) Continue mAb316P 75 mg Q2W, if the week 8 LDL-C is <100 mg/dL (2.59 mmol/L), or (2) Dose up-titrate to mAb316P 150 mg Q2W, if the week 8 LDL-C is ≧100 mg/dL (2.59 mmol/L).

To maintain the blind, the sites and the sponsor's operational team were blinded to dose modification.

### Concomitant Medications

Concomitant medications were kept to a minimum during the study. If considered necessary for the patient's welfare and unlikely to interfere with study drug, concomitant medications (other than those that are prohibited during the study) were permitted to be given at the discretion of the investigator, with a stable dose (when possible).

Addition of Concomitant Lipid-Modifying Treatment:

During the double-blind treatment period, addition of other LMTs was permitted only under certain conditions: (1) exceptional circumstances—overriding concerns (including, but not limited to, TG alert, below, posted by the central lab) warrant such changes, per the investigator's judgment, or (2) a confirmed TG alert—the patient meets the pre-specified TG alert (TG≧500 mg/dL [5.65 mmol/L). For a TG alert that has been confirmed by repeat testing, the investigator should perform investigations, manage the patient, and add other LMT per his/her medical judgment. For the above circumstances, lab alerts were sent. During the follow-up period, patients were permitted to resume their usual (pre-randomization) statin therapy and addition of other LMTs was permitted.

Permitted Medications:

Nutraceutical products or over-the-counter therapies that may affect lipids were allowed only if they had been used at a stable dose for at least 4 weeks before the screening visit, during the screening period, and maintained during the study. Examples of such nutraceutical products or over-the-counter therapies include omega-3 fatty acids at doses <1000 mg, plant stanols such as found in Benecol, flax seed oil, and psyllium.

Prohibited Medications:

Prohibited concomitant medications from the initial screening visit until the end of the study visit included the following: (1) Statins other than atorvastatin and rosuvastatin (provided as blinded medications), (2) EZE (other than that provided as blinded medication), (3) Fibrates, other than fenofibrate, and (4) Red yeast rice products.

### Study Endpoints

Baseline characteristics will include standard demography (e.g., age, race, weight, height, etc.), disease characteristics including medical history, and medication history for each patient.

Primary Efficacy Endpoint:

The primary efficacy endpoint was the percent change in calculated LDL-C from baseline to week 24, which is defined as: 100× (calculated LDL-C value at week 24−calculated LDL-C value at baseline)/calculated LDL-C value at baseline. The baseline calculated LDL-C value was the last LDL-C level obtained before the first double-blind study drug injection. The calculated LDL-C at week 24 will be the LDL-C level obtained within the week 24 analysis window and during the main efficacy period. The main efficacy period was defined as the time from the first double-blind study drug injection up to 21 days after the last double-blind study drug injection or up to the upper limit of the week 24 analysis window, whichever comes first.

All calculated LDL-C values (scheduled or unscheduled, fasting or not fasting) were used to provide a value for the primary efficacy endpoint if appropriate according to above definition. The analysis window used to allocate a time point to a measurement will be defined in the statistical analysis plan (SAP).

Secondary Efficacy Endpoints:

Secondary endpoints of the present study were as follows:

(1) The percent change in calculated LDL-C from baseline to week 12: similar definition and rules as for primary efficacy endpoint, except that the calculated LDL-C at week 12 was the LDL-C level obtained within the week 12 analysis window and during the 12-week efficacy period.

(2) The percent change in ApoB from baseline to week 24. Same definition and rules as for the primary endpoint.

(3) The percent change in non-HDL-C from baseline to week 24. Same definition and rules as for the primary endpoint.

(4) The percent change in total-C from baseline to week 24. Same definition and rules as for the primary endpoint.

(5) The percent change in ApoB from baseline to week 12. Same definition and rules as for the percent change in calculated LDL-C from baseline to week 12.

(6) The percent change in non-HDL-C from baseline to week 12. Same definition and rules as for the percent change in calculated LDL-C from baseline to week 12.

(7) The percent change in total-C from baseline to week 12. Same definition and rules as for the percent change in calculated LDL-C from baseline to week 12.

(8) The proportion of patients reaching LDL-C goal at week 24, i.e., LDL C<70 mg/dL (1.81 mmol/L) for patients with documented CHD, or non-CHD CVD, or diabetes mellitus with target organ damage, or <100 mg/dL (2.59 mmol/L) for patients with heFH or non-FH, without CHD or non-CHD CVD, but with a calculated 10-year fatal CVD risk SCORE ≧5%, or with moderate CKD, or with diabetes mellitus but no target organ damage, defined as: (number of patients whose calculated LDL-C value at week 24 reach LDL-C goal/number of patients in the (modified intent-to-treat [mITT population])*100, using definition and rules used for the primary endpoint.

(9) The percent change in Lp(a) from baseline to week 24. Same definition and rules as for the primary endpoint.

(10) The percent change in HDL-C from baseline to week 24. Same definition and rules as for the primary endpoint.

(11) The percent change in HDL-C from baseline to week 12. Same definition and rules as for the percent change in calculated LDL-C from baseline to week 12.

(12) The percent change in Lp(a) from baseline to week 12. Same definition and rules as for the percent change in calculated LDL-C from baseline to week 12.

(13) The percent change in fasting TG from baseline to week 24. Same definition and rules as for the primary endpoint.

(14) The percent change in fasting TG from baseline to week 12. Same definition and rules as for the percent change in calculated LDL-C from baseline to week 12.

(15) The percent change in ApoA-1 from baseline to week 24. Same definition and rules as for the primary endpoint.

(16) The percent change in ApoA-1 from baseline to week 12. Same definition and rules as for the percent change in calculated LDL-C from baseline to week 12.

(17) The proportion of patients reaching LDL-C goal at weeks 12, i.e., LDL-C<70 mg/dL (1.81 mmol/L) for documented CHD, or non-CHD CVD, or diabetes mellitus with target organ damage, or <100 mg/dL (2.59 mmol/L) for patients with heFH or non-FH, without CHD or non-CHD CVD, but with a calculated 10-year fatal CVD risk SCORE ≧5%, or with moderate CKD, or with diabetes mellitus but no target organ damage.

(18) The proportion of patients reaching LDL-C<100 mg/dL (2.59 mmol/L) at week 24.

(19) The proportion of patients reaching LDL-C<70 mg/dL (1.81 mmol/L) at week 24.

(20) The proportion of patients reaching LDL-C<100 mg/dL (1.81 mmol/L) at week 12

(21) The proportion of patients reaching LDL-C<70 mg/dL (2.59 mmol/L) at week 12

(22) The absolute change in calculated LDL-C (mg/dL and mmol/L) from baseline to weeks 12 and 24.

(23) The change in ratio ApoB/ApoA-1 from baseline to weeks 12 and 24.

(24) The proportion of patients with ApoB<80 mg/dL (0.8 mmol/L) at weeks 12 and 24.

(25) The proportion of patients with non-HDL-C<100 mg/dL at weeks 12 and 24.

The proportion of patients with calculated LDL-C<70 mg/dL (1.81 mmol/L) and/or 50% reduction in calculated LDL-C(if calculated LDL-C≧70 mg/dL [1.81 mmol/L]) at weeks 12 and 24.

Other Endpoints:

(1) Anti-mAB316P anti-drug-antibody status (positive/negative) and titers assessed throughout the study; (2) The percent change in high-sensitivity C-reactive protein (hs-CRP) from baseline to weeks 12 and 24; (3) The absolute change in homeostasis model assessment for insulin resistance (HOMA-IR) (%) from baseline to weeks 12 and 24; and (4) The absolute change in HbA1c (%) from baseline to weeks 12 and 24.

### Study Procedures

All laboratory samples were collected before the dose of study drug was administered. Blood samples for lipid panels were collected in the morning, in fasting condition (i.e., overnight, at least a 10-hour fast and refrain from smoking) for all clinic visits. Alcohol consumption within 48 hours and intense physical exercise within 24 hours preceding blood sampling were discouraged. Note: if the patient was not in fasting conditions, the blood sample was not collected and a new appointment was scheduled the day after (or as close as possible to this date) with a reminder to be fasted.

Total-C, HDL-C, TG, ApoB, ApoA-1, and Lp (a) were directly measured by a central laboratory. LDL-C was calculated using the Friedewald formula. If TG values exceeded 400 mg/dL (4.52 mmol/L) then the central lab reflexively measured (via the beta quantification method) the LDL-C rather than calculating it. Non-HDL-C was calculated by subtracting HDL-C from the total-C. Ratio ApoB/ApoA-1 was calculated.

Lipid Panel (Fasting):

Blood samples for the lipid panel (total-C, TG, HDL-C, and calculated LDL-C) were collected after at least a 10-hour fast at pre-specified time points.

Specialty Lipid Panel (Fasting): Blood samples for the specialty lipid panel (ApoB, ApoA-1, ApoB/ApoA-1 ratio, and Lp[a]) were collected after at least a 10-hour fast at pre-specified time points.

Blood Pressure and Heart Rate:

Blood pressure and heart rate were assessed at pre-specified time points. Blood pressure was preferably measured in sitting position under standardized conditions, approximately at the same time of the day, on the same arm, with the same apparatus (after the patient has rested comfortably in sitting position for at least 5 minutes). At the first screening visit, blood pressure was measured in both arms. The arm with the highest diastolic pressure was determined at this visit, and blood pressure was measured on this arm throughout the study. This highest value was recorded in the electronic case report form (eCRF). Heart rate was measured at the time of the measurement of blood pressure.

Physical Examination:

A thorough and complete physical examination, including height and weight, was performed at the baseline visit (visit 3). Physical exam with body weight was performed at pre-specified time points.

Body Weight and Height:

Body weight were obtained with the patient wearing undergarments or very light clothing and no shoes, and with an empty bladder. The same scale was preferably used throughout the study. The use of calibrated balance scales was recommended, if possible.

Electrocardiogram:

Electrocardiograms were performed before blood is drawn during visits that required blood draws. A standard 12-lead ECG was performed at pre-specified time points. The 12-lead ECGs were performed after at least 10 minutes rest and in the supine position. The electrodes were positioned at the same place, as much as possible, for each ECG recording throughout the study. The ECG were interpreted locally by the investigator. Each trace was analyzed in comparison with the screening recorded trace.

Laboratory Testing:

All laboratory samples were collected before the dose of study drug was administered. Samples for laboratory testing were collected at pre-specified time points and analyzed by a central laboratory during the study.

### Results

### Subject Disposition

A total of 355 patients were randomized into the 7 treatment arms, specifically:

(1) 57 patients in atorvastatin 40 mg;

(2) 55 patients in atorvastatin 20 mg+EZE;

(3) 57 patients in atorvastatin 20 mg+mAb316P;

(4) 47 patients in atorvastatin 80 mg;

(5) 45 patients in rosuvastatin 40 mg;

(6) 47 patients in atorvastatin 40 mg+EZE; and

(7) 47 patients in atorvastatin 40 mg+mAb316P.

Overall, 86.5%, 81.4%, and 88.6% of patients randomized to mAb316P add-on (3 and 7 combined), ezetimibe add-on (2 and 6 combined), or atorvastatin dose increase/switch to rosuvastatin (1, 4 and 5 combined), respectively, completed 24 weeks of double-blind treatment (defined as at least 22 weeks of treatment and Week 24 visit performed). Baseline characteristics were generally similar across the treatment groups as summarized in Tables 1A and 1B.

TABLE 1A
Baseline Characteristics (Entry Statin = Atorvastatin 20 mg [n = 169])

| | mAb316P 75/150 | | | | | |
|---|---|---|---|---|---|---|
| | mg + ATV 20 mg | | EZE 10 mg + ATV 20 mg | | ATV 40 mg | |
| | (n = 57) | | (n = 55) | | (n = 57) | |
| Age, years, mean (SD) | 62.2 | (10.0) | 65.7 | (9.0) | 63.0 | (9.9) |
| Male % (n) | 57.9 | (33) | 56.4 | (31) | 61.4 | (35) |
| Race, White % (n) | 84.2 | (48) | 87.3 | (48) | 87.7 | (50) |
| Ethnicity, | 16 | (28.1) | 13 | (23.6) | 15 | (26.3) |
| Hispanic/Latino, n (%) | | | | | | |
| BMI, kg/m², mean (SD) | 32.2 | (7.7) | 31.6 | (6.0) | 31.4 | (6.8) |
| HeFH % (n) | 10.5 | (6) | 0 | | 3.5 | (2) |
| CHD history, n (%) | 22 | (38.6) | 28 | (50.9) | 29 | (50.9) |
| CHD risk equivalent, n (%) | 16 | (28.1) | 16 | (29.1) | 19 | (33.3) |
| Hypertension % (n) | 77.2 | (44) | 81.8 | (45) | 80.7 | (46) |
| Type 2 diabetes % (n) | 57.9 | (33) | 52.7 | (29) | 54.4 | (31) |
| Use of lipid-lowering | 8 | (14.0) | 9 | (16.4) | 11 | (19.3) |
| therapy other than | | | | | | |
| statin, n (%) | | | | | | |
| LDL-C (calculated) | 103.9 | (34.9) | 100.4 | (29.5) | 100.3 | (29.8) |
| mean (SD), mg/dL | | | | | | |
| Non-HDL-C, | 131.4 ± 37.7 | | 128.6 ± 35.8 | | 129.7 ± 34.9 | |
| mean ± SD | | | | | | |
| Apo B, mean ± SD | 90.0 ± 21.9 | | 89.2 ± 22.6 | | 90.6 ± 22.8 | |
| Lp(a), median (Q1:Q3) | 24.0 | (7.0:78.0) | 21.0 | (11.0:48.0) | 16.0 | (6.0:52.0) |
| Fasting Triglycerides, | 134.0 | (100.0:169.0) | 124.0 | (92.0:177.0) | 126.0 | (91.0:192.0) |
| median (Q1:Q3) | | | | | | |

| | | | |
|---|---|---|---|
| HDL-C, mean ± SD | 47.2 ± 12.2 | 48.9 ± 11.3 | 51.2 ± 14.8 |

TABLE 1B
Baseline Characteristics (Entry Statin = Atorvastatin 40 mg [n = 186])

| | mAb316P 75/150 | | | | | | | |
|---|---|---|---|---|---|---|---|---|
| | mg + ATV 40 mg | | EZE 10 mg + | | | | | |
| | (n = 47) | | ATV 40 mg (n = 47) | | ATV 80 mg (n = 47) | | RSV 40 mg (n = 45) | |
| Age, years, mean (SD) | 64.2 | (10.4) | 63.9 | (10.3) | 63.2 | (10.9) | 57.5 | (10.0) |
| Male % (n) | 66.0 | (31) | 76.6 | (36) | 70.2 | (33) | 71.1 | (32) |
| Race, White % (n) | 91.5 | (43) | 91.5 | (43) | 87.2 | (41) | 73.3 | (33) |
| Ethnicity, | 6 | (12.8) | 5 | (10.6) | 6 | (12.8) | 6 | (13.3) |
| Hispanic/Latino | | | | | | | | |
| BMI, kg/m², mean (SD) | 29.8 | (5.4) | 30.8 | (5.9) | 30.2 | (6.0) | 30.8 | (6.9) |
| HeFH % (n) | 12.8 | (6) | 8.5 | (4) | 8.5 | (4) | 22.2 | (10) |
| CHD history, n (%) | 33 | (70.2) | 35 | (74.5) | 31 | (66.0) | 22 | (48.9) |
| CHD risk equivalent, n (%) | 10 | (21.3) | 15 | (31.9) | 16 | (34.0) | 8 | (17.8) |
| Hypertension % (n) | 76.6 | (36) | 78.7 | (37) | 78.7 | (37) | 73.3 | (33) |
| Type 2 diabetes % (n) | 53.2 | (25) | 34.0 | (16) | 53.2 | (25) | 40.0 | (18) |
| Use of lipid-lowering | 15 | (31.9) | 12 | (25.5) | 16 | (34.0) | 5 | (11.1) |
| therapy other than | | | | | | | | |
| statin, n (%) | | | | | | | | |
| LDL-C (calculated) | 116.4 | (37.4) | 98.9 | (29.2) | 108.6 | (37.5) | 109.8 | (39.0) |
| mean (SD), mg/dL | | | | | | | | |
| Non-HDL-C, | 144.5 ± 40.4 | | 124.5 ± 34.5 | | 136.2 ± 40.4 | | 140.0 ± 46.7 | |
| mean ± SD | | | | | | | | |
| Apo B, mean ± SD | 97.0 ± 25.5 | | 83.3 ± 17.0 | | 92.1 ± 24.6 | | 93.9 ± 26.6 | |
| Lp(a), median (Q1:Q3) | 21.0 | (7.0:67.0) | 32.0 | (9.0:58.0) | 43.5 | (8.0:89.0) | 43.0 | (10.0:98.0) |
| Fasting Triglycerides, | 120.0 | (98.0:185.0) | 113.0 | (84.0:155.0) | 122.0 | (85.0:172.0) | 116.0 | (88.0:199.0) |
| median (Q1:Q3) | | | | | | | | |

| | | | | |
|---|---|---|---|---|
| HDL-C, mean ± SD | 49.3 ± 14.5 | 46.5 ± 11.7 | 47.9 ± 15.1 | 50.0 ± 13.6 |

Persistent use of study drug injections (i.e., mAb316P/placebo, with compliance based on mean injection frequency) was high (>98%) across the pooled treatment groups. Over 70% of patients received all planned injections. Treatment injection exposure was similar among treatment groups, with a mean (SD) exposure of 22.0 (6.3) weeks in the pooled mAb316P add-on group, 22.1 (5.4) weeks in the pooled ezetimibe add-on group, and 22.7 (4.8) weeks in the pooled atorvastatin dose increase/switch to rosuvastatin group.

In this study, one patient in the atorvastatin 40 mg+EZE arm was randomized but did not receive study treatment for a reason related to IMP administration. Therefore the safety population contained 354 patients. Ten patients from the randomized population were excluded from the ITT Population due to a lack of post-baseline LDL-C assessments. A total of 15 patients from the randomized population were excluded from the mITT Population due to a lack of on-treatment LDL-C assessments.

### Efficacy Results

In general, demographic characteristics, baseline disease characteristics, baseline efficacy lipid parameters, LMT history and background LMT use were comparable among the treatment arms. Particularly, the mean baseline LDL-C values confirmed homogeneity at baseline for the 20 mg atorvastatin regimen with individual treatment arm means ranging from 100.3 mg/dL to 103.9 mg/dL in the 20 mg atorvastatin regimen. For the 40 mg atorvastatin regimen, the mean baseline LDL-C values trended higher in the mAb316P+atorvastatin 40 mg arm with a value of 116.4 mg/dL, and lower in the ezetimibe+atorvastatin 40 mg arm showing a value of 98.9 mg/dL.

The study included 2 atorvastatin dose regimens and 7 treatment arms. The five primary pairwise comparisons are defined within each atorvastatin regimen per IVRS/IWRS, as described in Table 2.

TABLE 2
Primary Pairwise Comparisons

| Atorvastatin Dose | |
|---|---|
| Regimen | Pairwise Comparison |
| 20 mg regimen | Comparison 1: mAb316P + atorva 20 mg arm vs. |
| | atorva 40 mg arm |
| | Comparison 2: mAb316P + atorva 20 mg arm vs. |
| | EZE + atorva 20 mg arm |
| 40 mg regimen | Comparison 3: mAb316P + atorva 40 mg arm vs. |
| | atorva 80 mg arm |
| | Comparison 4: mAb316P + atorva 40 mg arm vs. |
| | rosuva 40 mg arm |
| | Comparison 5: mAb316P + atorva 40 mg arm vs. |
| | EZE + atorva 40 mg arm |

To account for statistical testing of the five primary pairwise treatment comparisons described in Table 2, the alpha level is adjusted for multiplicity to 0.01 for each comparisons hereby controlling for the overall study alpha level.

The primary and key secondary efficacy analysis results are set forth in Tables 3 through 20. For clarification, the ITT analysis is defined for patients in the ITT population and includes all endpoint assessments in an analysis window, regardless of study treatment dosing status (i.e. includes post-treatment assessments). The on-treatment analysis is defined for patients in the mITT population and includes all endpoint assessments from the first double-blind study drug (capsule or injection, whichever comes first) up to 21 days after the last double-blind study drug injection or 3 days after the last capsule intake, whichever comes first (i.e. includes assessments in the efficacy treatment period). NOTE: P-values that are considered statistically significant as described in the hierarchical testing order at the 0.01 level are followed by an * in the Tables 3 through 20.

TABLE 3
The primary efficacy analysis for percent change from baseline
of calculated LDL-C to week 24 in the ITT Population

| | Compar- | Compar- | Compar- | Compar- | Compar- |
|---|---|---|---|---|---|
| | ison 1 | ison 2 | ison 3 | ison 4 | ison 5 |
| Delta | −39.1% | −23.6% | −49.2% | −32.6% | −31.4% |
| P-value | <0.0001* | 0.0004* | <0.0001* | <0.0001* | <0.0001* |

TABLE 4
The key secondary efficacy analyses for percent change from baseline
of calculated LDL-C to week 24 in the mITT Population

| | Compar- | Compar- | Compar- | Compar- | Compar- |
|---|---|---|---|---|---|
| | ison 1 | ison 2 | ison 3 | ison 4 | ison 5 |
| Delta | −42.5% | −24.9% | −52.8% | −35.0% | −33.4% |
| P-value | <0.0001* | 0.0002* | <0.0001* | <0.0001* | <0.0001* |

TABLE 5
The key secondary efficacy analyses for percent change from
baseline of calculated LDL-C to week 12 in the ITT Population

| | Compar- | Compar- | Compar- | Compar- | Compar- |
|---|---|---|---|---|---|
| | ison 1 | ison 2 | ison 3 | ison 4 | ison 5 |
| Delta | −39.8% | −25.8% | −36.0% | −27.3% | −20.9% |
| P-value | <0.0001* | <0.0001* | <0.0001* | <0.0001* | <0.0001* |

TABLE 6
The key secondary efficacy analyses for percent change from baseline
of calculated LDL-C to week 12 in the mITT Population

| | Compar- | Compar- | Compar- | Compar- | Compar- |
|---|---|---|---|---|---|
| | ison 1 | ison 2 | ison 3 | ison 4 | ison 5 |
| Delta | −44.5% | −26.6% | −36.3% | −27.7% | −20.2% |
| P-value | <0.0001* | <0.0001* | <0.0001* | <0.0001* | <0.0001* |

TABLE 7
The key secondary efficacy analyses for percent change
from baseline of Apo B to week 24 in the ITT Population

| | Compar- | Compar- | Compar- | Compar- | Compar- |
|---|---|---|---|---|---|
| | ison 1 | ison 2 | ison 3 | ison 4 | ison 5 |
| Delta | −29.3% | −23.6% | −38.4% | −30.9% | −27.6% |
| P-value | <0.0001* | <0.0001* | <0.0001* | <0.0001* | <0.0001* |

TABLE 8
The key secondary efficacy analyses for percent change from
baseline of Apo B to week 24 in the mITT Population

| | Compar- | Compar- | Compar- | Compar- | Compar- |
|---|---|---|---|---|---|
| | ison 1 | ison 2 | ison 3 | ison 4 | ison 5 |
| Delta | −32.6% | −25.1% | −38.3% | −29.8% | −26.4% |
| P-value | <0.0001* | <0.0001* | <0.0001* | <0.0001* | <0.0001* |

TABLE 9
The key secondary efficacy analyses for percent change from
baseline of non-HDL-C to week 24 in the ITT Population

| | Compar- | Compar- | Compar- | Compar- | Compar- |
|---|---|---|---|---|---|
| | ison 1 | ison 2 | ison 3 | ison 4 | ison 5 |
| Delta | −30.4% | −21.6% | −41.1% | −30.2% | −26.6% |
| P-value | <0.0001* | 0.0002* | <0.0001* | <0.0001* | <0.0001* |

TABLE 10
The key secondary efficacy analyses for percent change from
baseline of non-HDL-C to week 24 in the mITT Population

| | Compar- | Compar- | Compar- | Compar- | Compar- |
|---|---|---|---|---|---|
| | ison 1 | ison 2 | ison 3 | ison 4 | ison 5 |
| Delta | −33.0% | −22.4% | −43.6% | −32.1% | −27.3% |
| P-value | <0.0001* | <0.0001* | <0.0001* | <0.0001* | <0.0001* |

TABLE 11
The key secondary efficacy analyses for percent change from
baseline of Total-C to week 24 in the ITT Population

| | Compar- | Compar- | Compar- | Compar- | Compar- |
|---|---|---|---|---|---|
| | ison 1 | ison 2 | ison 3 | ison 4 | ison 5 |
| Delta | −23.1% | −15.8% | −28.9% | −21.9% | −18.4% |
| P-value | <0.0001* | <0.0001* | <0.0001* | <0.0001* | <0.0001* |

TABLE 12
The key secondary efficacy analyses for percent change
from baseline of Apo-B to week 12 in the ITT Population

| | Compar- | Compar- | Compar- | Compar- | Compar- |
|---|---|---|---|---|---|
| | ison 1 | ison 2 | ison 3 | ison 4 | ison 5 |
| Delta | −31.5% | −25.3% | −26.7% | −22.2% | −15.9% |
| P-value | <0.0001* | <0.0001* | <0.0001* | <0.0001* | <0.0001* |

TABLE 13
The key secondary efficacy analyses for percent change from
baseline of Non-HDL-C to week 12 in the ITT Population

| | Compar- | Compar- | Compar- | Compar- | Compar- |
|---|---|---|---|---|---|
| | ison 1 | ison 2 | ison 3 | ison 4 | ison 5 |
| Delta | −33.5% | −23.4% | −29.3% | −22.5% | −14.8% |
| P-value | <0.0001* | <0.0001* | <0.0001* | <0.0001* | 0.0001* |

TABLE 14
The key secondary efficacy analyses for percent change from
baseline of Total-C to week 12 in the ITT Population

| | Compar- | Compar- | Compar- | Compar- | Compar- |
|---|---|---|---|---|---|
| | ison 1 | ison 2 | ison 3 | ison 4 | ison 5 |
| Delta | −22.6% | −15.8% | −19.1% | −15.4% | −9.8% |
| P-value | <0.0001* | <0.0001* | <0.0001* | <0.0001* | 0.0015* |

TABLE 15
The key secondary efficacy analyses for proportion of
very high CV risk patients reaching calculated LDL-C <
70 mg/dL or high CV risk patients reaching calculated
LDL-C < 100 mg/dL at Week 24 in the ITT Population

| | Compar- | Compar- | Compar- | Compar- | Compar- |
|---|---|---|---|---|---|
| | ison 1 | ison 2 | ison 3 | ison 4 | ison 5 |
| Odds Ratio | 16.3 | 3.7 | 92.5 | 6.1 | 7.9 |
| P-value | <0.0001* | 0.018 | <0.0001* | 0.0044* | 0.0018* |

TABLE 16
The key secondary efficacy analyses for proportion of
very high CV risk patients reaching calculated LDL-C <
70 mg/dL or high CV risk patients reaching calculated
LDL-C < 100 mg/dL at Week 24 in the mITT Population

| | Compar- | Compar- | Compar- | Compar- | Compar- |
|---|---|---|---|---|---|
| | ison 1 | ison 2 | ison 3 | ison 4 | ison 5 |
| Odds Ratio | 24.6 | 5.0 | 130.8 | 8.5 | 10.8 |
| P-value | <0.0001* | 0.0184 | <0.0001* | 0.0025* | 0.0011* |

TABLE 17
The key secondary efficacy analyses for proportion
of patients reaching calculated LDL-C <
70 mg/dL at Week 24 in the ITT Population

| | Compar- | Compar- | Compar- | Compar- | Compar- |
|---|---|---|---|---|---|
| | ison 1 | ison 2 | ison 3 | ison 4 | ison 5 |
| Odds Ratio | 28.9 | 4.7 | 116.8 | 13.2 | 9.9 |
| P-value | <0.0001* | 0.0018 | <0.0001* | <0.0001* | 0.0004* |

TABLE 18
The key secondary efficacy analyses for proportion
of patients reaching calculated LDL-C <
70 mg/dL at Week 24 in the mITT Population

| | Compar- | Compar- | Compar- | Compar- | Compar- |
|---|---|---|---|---|---|
| | ison 1 | ison 2 | ison 3 | ison 4 | ison 5 |
| Odds Ratio | 28.8 | 4.8 | 162.1 | 19.8 | 13.9 |
| P-value | <0.0001* | 0.0054 | <0.0001* | <0.0001* | 0.0002* |

TABLE 19
The key secondary efficacy analyses for percent change
from baseline of Lp(a) to week 24 in the ITT Population

| | Compar- | Compar- | Compar- | Compar- | Compar- |
|---|---|---|---|---|---|
| | ison 1 | ison 2 | ison 3 | ison 4 | ison 5 |
| Delta | −3.4% | −13.0% | −21.1% | −25.9% | −31.0% |
| P-value | 0.552 | 0.0294 | 0.0004* | <0.0001* | <0.0001* |

TABLE 20
The key secondary efficacy analyses for percent change
from baseline of HDL-C to week 24 in the ITT Population

| | Compar- | Compar- | Compar- | Compar- | Compar- |
|---|---|---|---|---|---|
| | ison 1 | ison 2 | ison 3 | ison 4 | ison 5 |
| Delta | 2.9% | 4.9% | 2.9% | 2.0% | 5.6% |
| P-value | 0.3152 | 0.0973 | 0.4456 | 0.6086 | 0.1426 |

LDL-C results at week 24 are further summarized in Table 21.

TABLE 21
Effect of mAb316P on LDL-C at Week 24

| | Baseline Atorvastatin 20 mg | | | Baseline Atorvastatin 40 mg | | | |
|---|---|---|---|---|---|---|---|
| | | EZE + | mAb316P + | | | EZE + | mAb316P + |
| Randomized | ATV 40 | ATV 20 | ATV 20 | ATV 80 | RSV 40 | ATV 40 | ATV 40 |
| pts | n = 53 | n = 53 | n = 55 | n = 47 | n = 45 | n = 46 | n = 46 |
| ITT population | 53 | 53 | 55 | 47 | 45 | 46 | 46 |
| (n) | | | | | | | |
| Baseline (ITT), | 100.5 | 101.4 | 103.4 | 108.6 | 109.8 | 99.2 | 117.2 |
| mean (SD), | (30.9) | (29.3) | (34.9) | (37.5) | (39.0) | (29.4) | (37.4) |
| mg/dL | | | | | | | |
| W24 % change | −5.0 | −20.5 | −44.1 | −4.8 | −21.4 | −22.6 | −54.0 |
| from baseline, | (4.6) | (4.7) | (4.5) | (4.2) | (4.2) | (4.3) | (4.3) |
| LS mean (SE) | | | | | | | |
| Difference | −39.1 | −23.6 | | −49.2 | −32.6 | −31.4 | |
| mAb316P vs | (6.4) | (6.6) | | (6.1) | (6.0) | (6.1) | |
| comparator | | | | | | | |
| p-value | <.0001* | 0.0004* | | <.0001* | <.0001* | <.0001* | |
| mAb316P dose | | | 8.0 | | | | 20.9 |
| up-titration | | | | | | | |
| from 75 to 150 | | | | | | | |
| mg Q2W at | | | | | | | |
| W12, % pts | | | | | | | |

*Level of statistical significance: 0.01 folowing Bonferroni adjustment for multiplicity.
^{†}Very high-risk: <70 mg/dL; high-risk: <100 mg/dL.

The proportion of patients reaching LDL-C goal (i.e., a calculated LDL-C level of less than 70 mg/dL for very high risk CV patients, or a calculated LDL-C level of less than 100 mg/dL for high risk patients) at week 24 is summarized in Table 22.

TABLE 22
Combined Estimate for Proportion of Patients Reaching LDL-C Goal (%)

| | Baseline Atorvastatin 20 mg | | | Baseline Atorvastatin 40 mg | | | |
|---|---|---|---|---|---|---|---|
| | | EZE + | mAb316P + | | | EZE + | mAb316P + |
| Randomized | ATV 40 | ATV 20 | ATV 20 | ATV 80 | RSV 40 | ATV 40 | ATV 40 |
| pts | n = 53 | n = 53 | n = 55 | n = 47 | n = 45 | n = 46 | n = 46 |
| Proportion of | 34.5 | 66.5 | 87.2 | 15.4 | 62.2 | 65.1 | 82.4 |
| Patients | | | | | | | |
| Reaching | | | | | | | |
| LDL-C goal (%) | | | | | | | |
| p-value | <0.0001 | 0.0180 | | <0.0001 | 0.0044 | 0.0018 | |
| (mAb316P vs | | | | | | | |
| comparator) | | | | | | | |

The change in LDL-C levels in the various treatment groups over time is summarized in Table 23.

TABLE 23
Calculated LDL-C Over Time

| | | | Calculated LDL-C LS mean (SE) | | |
|---|---|---|---|---|---|
| Time | Baseline | | Value | Change from | % Change from |
| Point | Atorvastatin | Treatment | (mg/dL) | Baseline | Baseline |
| Baseline | 20 mg | ATV 40 | 100.5 (4.2) | NA | NA |
| Week 4 | | | 88.9 (3.6) | −12.9 (3.6) | −9.9 (3.4) |
| Week 8 | | | 86.7 (4.1) | −15.1 (4.1) | −12.6 (3.8) |
| Week 12 | | | 89.9 (4.3) | −11.9 (4.3) | −8.5 (3.9) |
| Week 16 | | | 90.9 (4.3) | −10.9 (4.3) | −8.2 (3.9) |
| Week 24 | | | 93.9 (4.7) | −7.9 (4.7) | −5.0 (4.6) |
| Baseline | 20 mg | EZE + | 101.4 (4.0) | NA | NA |
| Week 4 | | ATV 20 | 74.3 (3.6) | −27.5 (3.6) | −25.6 (3.5) |
| Week 8 | | | 75.0 (4.1) | −26.8 (4.1) | −26.2 (3.8) |
| Week 12 | | | 78.5 (4.3) | −23.3 (4.3) | −22.6 (3.9) |
| Week 16 | | | 78.7 (4.4) | −23.1 (4.4) | −22.0 (3.9) |
| Week 24 | | | 81.0 (4.8) | −20.8 (4.8) | −20.5 (4.7) |
| Baseline | 20 mg | mAb316P + | 103.4 4.7 | NA | NA |
| Week 4 | | ATV 20 | 46.0 (3.5) | −55.8 (3.5) | −53.3 (3.4) |
| Week 8 | | | 50.4 (4.1) | −51.4 (4.1) | −51.2 (3.8) |
| Week 12 | | | 52.8 (4.2) | −49.0 (4.2) | −48.4 (3.8) |
| Week 16 | | | 47.0 (4.3) | −54.8 (4.3) | −53.8 (3.9) |
| Week 24 | | | 54.3 (4.7) | −47.5 (4.7) | −44.1 (4.5) |
| Baseline | 40 mg | ATV 80 | 108.6 (5.5) | NA | NA |
| Week 4 | | | 93.4 (2.9) | −15.3 (2.9) | −11.7 (2.7) |
| Week 8 | | | 91.5 (3.2) | −17.2 (3.2) | −13.1 (3.1) |
| Week 12 | | | 90.8 (3.4) | −17.9 (3.4) | −14.5 (3.2) |
| Week 16 | | | 97.4 (3.8) | −11.3 (3.8) | −8.0 (3.4) |
| Week 24 | | | 100.0 (4.5) | −8.7 (4.5) | −4.8 (4.2) |
| Baseline | 40 mg | RSV 40 | 109.8 (5.8) | NA | NA |
| Week 4 | | | 83.5 (3.0) | −25.2 (3.0) | −22.4 (2.8) |
| Week 8 | | | 85.8 (3.3) | −22.9 (3.3) | −21.0 (3.2) |
| Week 12 | | | 82.4 (3.5) | −26.3 (3.5) | −23.3 (3.2) |
| Week 16 | | | 90.0 (3.8) | −18.7 (3.8) | −15.7 (3.5) |
| Week 24 | | | 85.6 (4.5) | −23.1 (4.5) | −21.4 (4.2) |
| Baseline | 40 mg | EZE + | 99.2 (4.3) | NA | NA |
| Week 4 | | ATV40 | 72.9 (3.0) | −35.8 (3.0) | −32.2 (2.8) |
| Week 8 | | | 75.7 (3.3) | −33.0 (3.3) | −28.9 (3.1) |
| Week 12 | | | 76.5 (3.5) | −32.2 (3.5) | −29.7 (3.2) |
| Week 16 | | | 79.0 (3.8) | −29.7 (3.8) | −28.2 (3.5) |
| Week 24 | | | 85.1 (4.6) | −23.6 (4.6) | −22.6 (4.3) |
| Baseline | 40 mg | mAb316P + | 117.2 (5.5) | NA | NA |
| Week 4 | | ATV 40 | 45.5 (2.9) | −63.2 (2.9) | −56.5 (2.8) |
| Week 8 | | | 45.3 (3.3) | −63.4 (3.3) | −55.7 (3.2) |
| Week 12 | | | 51.8 (3.5) | −56.9 (3.5) | −50.5 (3.2) |
| Week 16 | | | 44.4 (3.9) | −64.3 (3.9) | −56.9 (3.5) |
| Week 24 | | | 46.4 (4.6) | −62.3 (4.6) | −54.0 (4.3) |

As summarized in Table 23, mAb316P significantly reduced LDL-C levels versus all other comparators (p<0.001 for each comparison), and maintained this reduction consistently from Week 4 to Week 24. Reductions were consistent by on-treatment and pattern-mixture analysis methods, and using LDL-C measured by beta-quantification. mAb316P dose was increased from 75 mg to 150 mg Q2W at Week 12 in 8.0% and 20.9% of patients on baseline atorvastatin 20 mg or 40 mg, respectively. Overall, the majority (86%) of patients in the mAb316P add-on groups (with at least 1 injection post-randomization at Week 12) were maintained on the 75 mg Q2W dose after Week 12 as their Week 8 LDL-C was below the pre-defined threshold (<70 or <100 mg/dL depending on CVD risk); LDL-C reductions were consistent over time in these patients.

For patients receiving mAb316P added to background atorvastatin 20 mg and 40 mg, the combined proportion of very-high and high CVD-risk patients achieving protocol predefined LDL-C goals of <70 mg/dL and <100 mg/dL, respectively, at Week 24, was >80% when using calculated LDL-C values. Furthermore, achievement of the more stringent LDL-C goal of <70 mg/dL was achieved by 77-79% of patients in the mAb316P add-on groups, when using calculated LDL-C values. When using LDL-C values measured by beta-quantification, achievement of LDL-C<70 mg/dL or <100 mg/dL was similar to when calculated LDL-C was used for most groups. mAb316P reduced apolipoprotein B and non-high-density lipoprotein cholesterol (non-HDL-C) from baseline to Week 24 versus all comparators regardless of background atorvastatin regimen, and significantly reduced Lp(a) when compared with all comparators on a background of atorvastatin 40 mg (all p<0.001).

The efficacy results from this Example demonstrate that the addition of a PCSK9 antagonist (e.g., mAb316P) to moderate dose statin therapy (e.g., atorvastatin 20 mg or 40 mg daily) produced greater and more pronounced lipid lowering efficacy than treatment alternatives such as: (a) increasing the patient's daily statin dose (e.g., increasing daily atorvastatin from 20 mg to 40 mg or from 40 mg to 80 mg); (b) adding Ezetimibe to the patient's existing moderate dose statin therapy; or (c) switching to a different statin (e.g., switching to rosuvastatin).

### Safety Results

A summary of safety results are presented for the treatment groups of pooled statin dose regimens, with the intent to maximize efforts to detect potential safety signals. For the pooled data, three treatment groups are formed by combining treatment arms regardless of the atorvastatin doses as follows:

(1) The double-dose statin treatment group: pooling atorva 40 mg arm, atorva 80 mg arm, and rosuva 40 mg arm;

(2) EZE treatment group: pooling EZE+atorva 20 mg arm and EZE+atorva 40 mg arm;

(3) mAb316P treatment group: pooling mAb316P+atorva 20 mg arm and mAb316P+atorva 40 mg arm.

A total of 354 patients were randomized and received at least a partial dose of study treatment (Safety Population). A high-level safety summary of adverse events and events of interest is as follows.

Treatment-emergent SAEs occurred in 8 (5.4%) patients in the double-dose statin treatment group, 7 (6.9%) patients in the pooled EZE treatment group, and 4 (3.8%) patients in the pooled mAb316P treatment group.

Two patient deaths were reported in the study; both patients were in the pooled EZE treatment group.

A total of 19 patients discontinued study treatment early due to a treatment emergent adverse event (TEAE), specifically 8 (5.4%) patients in the double-dose statin treatment group, 4 (4.0%) patients in the pooled EZE treatment group, and 7 (6.7%) patients in the pooled mAb316P treatment group.

TEAEs occurred in 95 (63.8%) patients in the double-dose statin treatment group, 65 (64.4%) patients in the pooled EZE treatment group, and 68 (65.4%) patients in the pooled mAb316P treatment group.

The SOCs with a higher frequency in the pooled mAb316P as compared to both the double-dose statin and the pooled EZE treatment groups are: (a) “Musculoskeletal and connective tissue disorders”, with 19 (12.8%) patients in the double-dose statin treatment group, 13 (12.9%) patients in the EZE treatment group, and 24 (23.1%) patients in the mAb316P treatment group; (b) “Nervous system disorders”, with 13 (8.7%) patients in the double-dose statin treatment group, 6 (5.9%) patients in the EZE treatment group, and 10 (9.6%) patients in the mAb316P treatment group; (c) “Injury, poisoning and procedural complications,” with 19 (12.8%) patients in the double-dose statin treatment group, 12 (11.9%) patients in the EZE treatment group, and 15 (14.4%) patients in the mAb316P treatment group; (d) “Neoplasms benign, malignant and unspecified (incl cysts and polyps),” with 3 (2.0%) patients in the double-dose statin treatment group, 0 patients in the EZE treatment group, and 3 (2.9%) patients in the mAb316P treatment group. Of note, the 3 TEAEs in the alircoumab treatment group included a single case each of basal cell carcinoma, seborrhoeic keratitis, and squamous cell carcinoma of skin.

The SOCs with a higher frequency in both the double-dose statin and pooled EZE treatment groups compared to the pooled mAb316P treatment group included: Infections and infestations, Blood and lymphatic system disorders, Metabolism and nutrition disorders, Ear and labyrinth disorders, Cardiac disorders, Respirator, thoracic and mediastinal disorders, Gastrointestinal disorders, Hepatobiliary disorders, Skin and subcutaneous tissue disorders, General disorders and administration site conditions, and Investigations.

The most frequent TEAEs (reported in at least three patients) in the pooled mAb316P group are: Back pain (7 patients), Nasopharyngitis (5), Upper respiratory tract infection (5), Hypertension (5), and Headache (4), Muscle spasms (4), Influenza (3), Urinary tract infection (3).

For TEAEs of special interest (AESIs), results are presented by pre-defined SMQ preferred term groupings:

(1) Treatment-emergent injection site reactions (ISRs) occurred in 3 (2.0%) patients in the double-dose statin treatment group, 3 (3.0%) patients in the pooled EZE treatment group, and 3 (2.9%) patients in the pooled mAb316P treatment group;

(2) General Allergic TEAEs obtained through SMQ of the term “Hypersensitivity” occurred in 6 (4.0%) patients in the double-dose statin treatment group, 5 (5.0%) patients in the pooled EZE treatment group, and 2 (1.9%) patients in the pooled mAb316P treatment group;

(3) Treatment-emergent neurologic disorders occurred in 3 (2.0%) patients in the double-dose statin treatment group, 1 (1.0%) patients in the pooled EZE treatment group, and 3 (2.9%) patients in the pooled mAb316P treatment group;

(4) There were no treatment-emergent neurocognitive disorders reported in the safety population;

(5) A total of 4 patients were adjudicated positively for treatment-emergent cardiovascular events, and all four patients reported the event “ischemia driven coronary revascularization procedure”. The 4 events adjudicated positively were collected from 1 (0.7%) patient in the double-dose statin treatment group, 1 (1.0%) patient in the pooled EZE treatment group and 2 (1.9%) patients in the pooled mAb316P treatment group.

A baseline positive anti-drug antibody (“ADA”) response was observed in one patient (1.0%) in the pooled mAb316P add-on group, but also in the control treatment groups: three patients (3.2%) in the pooled ezetimibe add-on group and one patient (0.7%) in the pooled atorvastatin dose increase/switch to rosuvastatin group. These results indicate either a high serum background or pre-existing immunoreactivity and not a treatment-emergent ADA response. Treatment-emergent ADA positive responses were observed in 5 of 99 patients (5.1%) in the pooled mAb316P add-on group. Of these five patients, three had persistent responses, one had a transient response and the other had an indeterminate response. One out of the three patients with positive ADA status at the Week 24 time point had positive mAb316P-neutralizing antibody status. One patient in the pooled statin dose increase/switch to rosuvastatin group also had a treatment-emergent positive ADA response. Overall, immunogenicity was low and ADA positivity did not have an effect on the LDL-C lowering efficacy of mAb316P during this study, nor were any specific clinical events considered related to development of ADAs.

### Conclusions

This study demonstrated that among patients at high CVD risk treated with stable atorvastatin, adding mAb316P to a background of atorvastatin provided greater reductions in LDL-C levels compared with adding ezetimibe, doubling the atorvastatin dose, or switching to rosuvastatin. Specifically, among the atorvastatin 20 mg and 40 mg regimens, respectively, add-on mAb316P reduced LDL-C levels by 44.1% and 54.0%, add-on ezetimibe reduced LDL-C levels by 20.5% and 22.6%, doubling of atorvastatin dose reduced LDL-C levels by 5.0% and 4.8%, and switching atorvastatin 40 mg to rosuvastatin 40 mg reduced LDL-C levels by 21.4%. Most (˜80%) mAb316P-treated patients maintained their 75 mg Q2W regimen. The analysis of the primary endpoint was consistent regardless of the analysis method used (ITT, on-treatment, pattern-mixture) demonstrating the robustness of the results.

A unique aspect of this study design was that it employed a treat-to-goal dosing strategy, whereby the mAb316P dose was increased depending on individual patient response to treatment. While LDL-C treatment goals are at present no longer recommended by the ACC/AHA Lipid Treatment Guidelines, other US and international guidelines and recommendations continue to support the use of LDL-C treatment goals. A greater proportion of patients received mAb316P dose increase in the atorvastatin 40 mg per day group versus the atorvastatin 20 mg per day group. Reasons for this may be twofold: (1) Patients in the atorvastatin 40 mg group had a somewhat higher mean LDL-C level at baseline (116 vs 104 mg/dL) and (2) a greater proportion of patients on the higher atorvastatin dose of 40 mg per day presented with a history of CVD that, per protocol, required more aggressive LDL-C treatment goals.

mAb316P significantly improved protocol predefined LDL-C goal achievement versus all comparators, with 87.2% to 84.6% of patients in the mAb316P add-on groups achieving their LDL-C goals (<70 mg/dL or <100 mg/dL, depending on risk) versus comparators. Furthermore, 77.2% to 79.2% of patients in the mAb316P add-on groups achieved the more stringent LDL-C goal of <70 mg/dL (compared with: 50.3% to 54.2% with ezetimibe add-on, 10.2% to 16.0% with atorvastatin dose increase, and 42.2% with rosuvastatin switch). The above figures were derived using LDL-C calculated using the Friedewald equation; when measured LDL-C was used (via beta-quantification), achievement of LDL-C goals (<70 mg/dL or <100 mg/dL) followed a similar pattern.

mAb316P also significantly reduced apolipoprotein B, non-HDL-C versus active the comparators, and also reduced Lp(a) by 23.6 to 30.8%, similar to previous reports.

The LDL-C reductions observed with ezetimibe add-on groups (20.5-22.6%) and atorvastatin dose increase groups (5.0-4.8%) were consistent with previous reports. The 21.4% reduction in LDL-C levels when switching from atorvastatin 40 mg per day to rosuvastatin 40 mg per day was higher than the ˜8% reduction, as reported elsewhere.

Regarding safety, the incidence of TEAEs was comparable across all dosing groups, with a similar number of discontinuations because of TEAEs. The occurrence of allergic events, neurologic events, or injection site reactions was low. Development of antibodies to mAb316P (ADAs) were observed in only four patients following mAb316P treatment; these were mAb316P-neutralizing in one patient. Presence of ADAs did not affect overall efficacy or safety. Overall, the safety findings of this study were comparable with other clinical trials involving mAb316P and trials of other PCSK9 inhibitors.

In conclusion, in patients with very high or high CV risk not achieving LDL-C goals of <70 mg/dL or <100 mg/dL, respectively, with commonly used doses of atorvastatin, mAb316P as add-on to atorvastatin 20 mg or 40 mg produced significantly greater LDL-C reductions at Week 24 versus addition of ezetimibe, doubling the atorvastatin dose, or switch to rosuvasatin. The LDL-C lowering effect with mAb316P was seen from Week 4 and was maintained until the end of treatment.

### Example 3

### A Randomized, Double-Blind Study of the Efficacy and Safety of an Anti-PCSK9 Antibody (“mAb316P”) Added-on to Rosuvastatin Versus Ezetimibe Added-on to Rosuvastatin Versus Rosuvastatin Dose Increase in Patients Who are not Controlled on Rosuvastatin

### Introduction

The objective of the present study was to compare mAb316P as add-on therapy to submaximal doses of rosuvastatin in comparison with ezetimibe (EZE) as add-on therapy to submaximal doses of rosuvastatin, or in comparison with doubling the rosuvastatin dose in patients at high cardiovascular (CV) risk who have failed to reach their LDL-C treatment goal and require additional pharmacological management, with the exception of EZE, which was an active comparator in the study. The definition of high CV risk in this study is based on existing guidelines (ESC/EAS Guidelines for the management of dyslipidaemias, Executive summary of the Third Report of the National Cholesterol Education Program 2001).

Maximizing the dose of rosuvastatin is also a treatment option for patients who have failed to reach their LDL-C treatment goal (ESC/EAS Guidelines for the management of dyslipidaemia). Ezetimibe was selected as a comparator arm because it has been recommended as a treatment option for use in combination with statins.

The present study was double-blind and each patient received an injection Q2W and 2 oral capsules daily to maintain the double-blind.

### Study Objectives

The primary objective of this study was to evaluate the reduction of LDL-C by mAb316P as add-on therapy to rosuvastatin in comparison with EZE as add-on therapy to rosuvastatin, and in comparison with doubling the rosuvastatin dose, after 24 weeks of treatment in patients with hypercholesterolemia at high CV risk. The secondary objectives of this study were: (a) to evaluate the reduction of LDL-C by mAb316P 75 mg as add-on therapy to rosuvastatin in comparison with EZE as add-on therapy to rosuvastatin, or in comparison with doubling of the rosuvastatin dose after 12 weeks of treatment; (b) to evaluate the effect of mAb316P on other lipid parameters (e.g., ApoB, non-HDL-C, total-C, Lp(a), HDL-C, TG levels, ApoA-1, etc.); (c) to evaluate the safety and tolerability of mAb316P; and (d) to evaluate the development of anti-mAb316P antibodies.

### Study Design

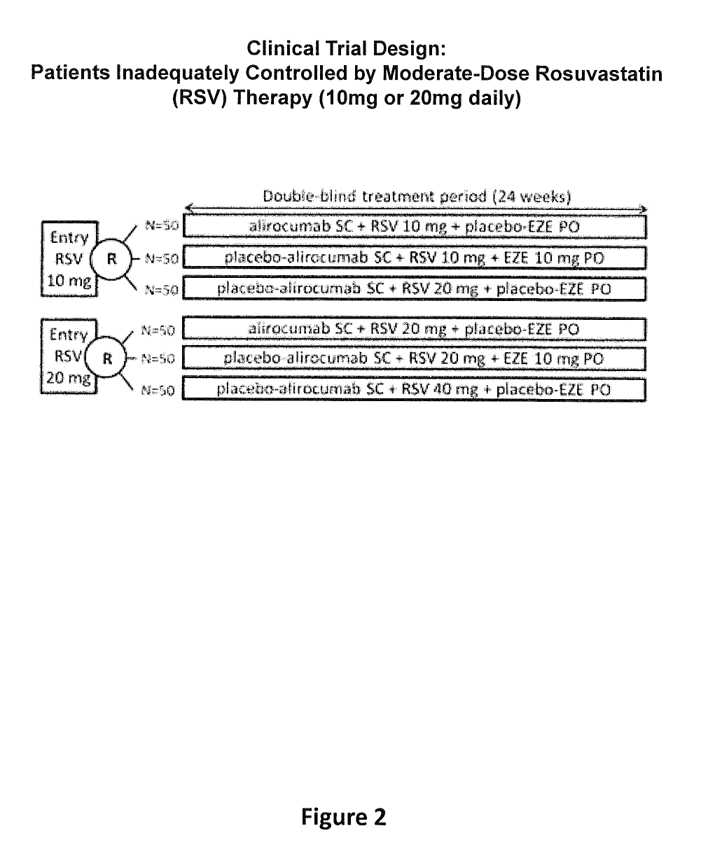

The present study was a randomized, double-blind, active-comparator, parallel-group study in patients at high CV risk with non-FH or heFH who are not adequately controlled with rosuvastatin (10 mg or 20 mg) with or without other lipid-modifying therapy (LMT) (excluding EZE). The study design is illustrated in FIG. 2. Patients who entered the study were taking either rosuvastatin 10 mg or rosuvastatin 20 mg. The former patients were randomized to 1 of 3 treatment arms (arms 1 to 3); the latter patients were randomized to 1 of 3 treatment arms (arms 4 to 6). The treatment arms are as follows:

Patients on a 10 mg Rosuvastatin Regimen:

(1) mAb316P+rosuvastatin 10 mg+placebo-EZE; (2) Placebo-mAb316P+rosuvastatin 20 mg+placebo-EZE; and (3) Placebo-mAb316P+rosuvastatin 10 mg+EZE 10 mg.

Patients on a 20 mg Rosuvastatin Regimen:

(4) mAb316P+rosuvastatin 20 mg+placebo-EZE; (5) Placebo-mAb316P+rosuvastatin 40 mg+placebo-EZE; and (6) Placebo-mAb316P+rosuvastatin 20 mg+EZE 10 mg.

Within each rosuvastatin regimen, randomization was stratified according to whether the patient has a prior history of either myocardial infarction (MI) or ischemic stroke (yes/no).

The present study consisted of:

(A) a screening period of up to 2 weeks, including an intermediate visit during which the patient or caregiver was trained to self-inject/inject using a dose of placebo-mAb316P. Patients who had been on a stable rosuvastatin dose for at least 4 weeks before the screening visit 1 (day −7) were screened for study eligibility at visit 1 (day −7 to 1). At the discretion of the investigator, patients who: (i) had not been on a stable dose of rosuvastatin (10 mg or 20 mg) for 4 weeks, (ii) were being switched from another statin to rosuvastatin, or (iii) were not on a statin but should have been according to local guidance may undergo an open-label 4-week rosuvastatin (10 mg or 20 mg) run-in period between the pre-screening visit 1a (day −42) and visit 1. The run-in dose of rosuvastatin (10 mg or 20 mg) was based on the medical judgment of the study physician.

(B) a double-blind treatment period of 24 weeks. Each patient received a SC injection Q2W (mAb316P or placebo-mAb316P) and took 2 oral blinded medications daily (rosuvastatin and EZE or placebo-EZE). The first injection of mAb316P or placebo-mAb316P was administered at the clinical site on day 1, after study assessments had been completed, and as soon as possible after the patient is randomized into the study. The patient/caregiver administered subsequent injections outside of the clinic according to the dosing schedule. On days where the clinic study visit coincided with dosing, the dose of study drugs (injectable and oral) was administered after all study assessments had been performed and all laboratory samples collected. The last dose of mAb316P or placebo-mAb316P was administered at week 22. The last dose of daily oral study drugs was administered at week 24. At week 12, based on their baseline CV risk, certain patients randomized to mAb316P, in a blinded manner, had their dose increased in accordance with the following:

(1) Patients with heFH or Non-FH and a History of Documented CHD, or Non-CHD CVD, or Diabetes Mellitus with Target Organ Damage:

(a) Continue mAb316P 75 mg Q2W, if the week 8 LDL-C is <70 mg/dL (1.8 mmol/L), or (b) Dose up-titrate to mAb316P 150 mg Q2W, if the week 8 LDL-C is ≧70 mg/dL (1.8 mmol/L);

(2) Patients with heFH or Non-FH, without CHD or Non-CHD CVD, but with a Calculated 10-Year Fatal CVD Risk SCORE ≧5%, or with Moderate CKD, or with Diabetes Mellitus but No Target Organ Damage:

(a) Continue mAb316P 75 mg Q2W, if the week 8 LDL-C is <100 mg/dL (2.59 mmol/L), or (b) Dose up-titrate to mAb316P 150 mg Q2W, if the week 8 LDL-C is ≧100 mg/dL (2.59 mmol/L). Lipid results were blinded from specimens obtained after randomization (including week 8). The continuation of the 75 mg dose or dose up-titration to the 150 mg dose was done using an automated process without site or patient awareness.

(C) A follow-up period of 8 weeks.

Patients were asked to follow a stable diet (the National Cholesterol Education Program Adult Treatment Panel III Therapeutic Lifestyle Changes [NCEP ATP III-TLC] diet or equivalent diet) from screening to the end of study visit. The addition of other LMT was not permitted during the double-blind treatment period except under certain conditions.

### Patient Selection

The study population consisted of patients with hypercholesterolemia and established CHD or non-CHD CVD (defined below), or who were at high risk for CVD due other factors and who were not adequately controlled with a 10 mg or 20 mg daily dose of rosuvastatin, with or without other LMT, except EZE.

Inclusion Criteria: The patients enrolled in this study met conditions 1a or 1 b (below) to be eligible for inclusion in the study:

1a. Patients with screening (visit 1) LDL-C≧70 mg/dL (1.81 mmol/L) who are not adequately controlled with a 10 mg or 20 mg stable daily dose of rosuvastatin for at least 4 weeks before the screening visit (visit 1), with or without other LMT (excluding EZE). Patients with heFH or non-FH must also have a history of documented CHD (defined below), or non-CHD CVD (defined below), or diabetes mellitus with target organ damage; OR

1 b. Patients with screening (visit 1) LDL-C≧100 mg/dL (2.59 mmol/L) who are not adequately controlled with a 10 mg or 20 mg daily dose of rosuvastatin for at least 4 weeks before the screening visit (visit 1), with or without other LMT (excluding EZE). Patients must also have heFH, or have non-FH, without CHD or non-CHD CVD, but with a calculated 10-year fatal CVD risk SCORE ≧5%, or with moderate CKD, or with diabetes mellitus but no target organ damage.

Note: Diagnosis of heFH was made either by genotyping or by clinical criteria.

Definitions for CHD, Non-CHD CVD, and Other Risk Factors:

A. A documented history of CHD (includes 1 or more of the following): i. Acute MI; ii. Silent MI; iii. Unstable angina; iv. Coronary revascularization procedure (e.g., percutaneous coronary intervention [PCI] or coronary artery bypass graft surgery [CABG]); and/or v. Clinically significant CHD diagnosed by invasive or non-invasive testing (such as coronary angiography, stress test using treadmill, stress echocardiography or nuclear imaging).

B. Non-CHD CVD (includes 1 or more of the following criteria): i. Documented previous ischemic stroke with a focal ischemic neurological deficit that persisted more than 24 hours, considered as being of atherothrombotic origin. CT or MRI is performed to rule out hemorrhage and non-ischemic neurological disease; ii. Peripheral arterial disease; iii. Abdominal aortic aneurysm; iv. Atherosclerotic renal artery stenosis; and/or v. Carotid artery disease (transient ischemic attacks or >50% obstruction of a carotid artery)

C. Other Risk Factors: i. Documented moderate CKD as defined by 30≦eGFR<60 mL/min/1.73 m2 for 3 months or more, including the screening visit; ii. Type 1 or type 2 diabetes mellitus with or without target organ damage (i.e., retinopathy, nephropathy, microalbuminuria); iii. A calculated 10-year fatal CVD risk SCORE ≧5% (ESC/EAS Guidelines for the management of dyslipidemias, Conroy et al., 2003, Eur. Heart J. 24:987-1003).

Exclusion Criteria:

Patients who met any of the following criteria were excluded from the study:

1. LDL-C<70 mg/dL (<1.81 mmol/L) at the screening visit (week −2) in patients with history of documented CHD or non-CHD CVD.

2. LDL-C<100 mg/dL (<2.59 mmol/L) at the screening visit (week −2) in patients without history of documented CHD or non-CHD CVD, but with other risk factors.

3. Homozygous FH (clinically or previous genotyping).

4. Currently taking a statin that is not rosuvastatin taken daily at 10 mg or 20 mg.

5. Currently taking EZE or had received EZE within 4 weeks of screening visit 1 (week −2).

6. Not on a stable dose of allowable LMT (excluding EZE) for at least 4 weeks and/or fenofibrate for at least 6 weeks prior to the screening visit (week −2) or from screening to randomization, as applicable.

7. Use of fibrates, other than fenofibrate within 6 weeks of the screening visit (week −2) or between screening and randomization visits.

8. Use of nutraceutical products or over-the-counter therapies that may affect lipids and which the dose amount has not been stable for at least 4 weeks prior to the screening visit (week −2), or between screening and randomization visits.

9. Use of red yeast rice products within 4 weeks of the screening visit (week −2) or between screening and randomization visits.

10. Patient who has received plasmapheresis treatment within 2 months prior to the screening visit (week −2), or has plans to receive it during the study.

11. Recent (within 3 months prior to the screening visit [week −2]) MI, unstable angina leading to hospitalization, percutaneous coronary intervention (PCI), coronary artery bypass graft surgery (CABG), uncontrolled cardiac arrhythmia, stroke, transient ischemic attack, carotid revascularization, endovascular procedure or surgical intervention for peripheral vascular disease.

12. Planned to undergo scheduled PCI, CABG, carotid or peripheral revascularization during the study.

13. Systolic blood pressure >160 mm Hg or diastolic blood pressure >100 mm Hg at screening visit and/or randomization visit.

14. History of New York Heart Association (NYHA) Class III or IV heart failure within the past 12 months.

15. Known history of hemorrhagic stroke.

16. Age <18 years or legal age of majority at the screening visit (week −2), whichever is greater.

17. Patients not previously instructed on a cholesterol-lowering diet prior to the screening visit (week-2).

18. Newly diagnosed (within 3 months prior to randomization visit [week 0]) or poorly controlled (hemoglobin A1c [HbA1c]>8.5%) diabetes.

19. Presence of any clinically significant uncontrolled endocrine disease known to influence serum lipids or lipoproteins. Note: patients on thyroid replacement therapy can be included if the dosage of thyroxine has been stable for at least 12 weeks prior to screening and the thyroid-stimulating hormone (TSH) level is within the normal range of the central laboratory at the screening visit.

20. History of bariatric surgery within 12 months prior to the screening visit (week −2).

21. Unstable weight defined by a variation >5 kg within 2 months prior to the screening visit (week −2).

22. Known history of loss of function of PCSK9 (i.e., genetic mutation or sequence variation).

23. Use of systemic corticosteroids, unless used as replacement therapy for pituitary/adrenal disease with a stable regimen for at least 6 weeks prior to randomization. Note: topical, intra-articular, nasal, inhaled and ophthalmic steroid therapies are not considered as “systemic” and are allowed.

24. Use of continuous estrogen or testosterone hormone replacement therapy unless the regimen has been stable in the past 6 weeks prior to the screening visit (week −2) and no plans to change the regimen during the study.

25. History of cancer within the past 5 years, except for adequately treated basal cell skin cancer, squamous cell skin cancer, or in situ cervical cancer.

26. Known history of HIV positive.

27. Patient who has taken any active investigational drugs within 1 month or 5 half-lives, whichever is longer.

28. Patient who has previously participated in any clinical trial of mAb316P or any other anti-PCSK9 monoclonal antibody.

29. Conditions/situations such as: (A) Any clinically significant abnormality identified at the time of screening that in the judgment of the Investigator or any sub-investigator would preclude safe completion of the study or constrain endpoints assessment such as major systemic diseases, patients with short life expectancy; or (B) Patients considered by the investigator or any sub-investigator as inappropriate for this study for any reason, e.g.: (i) Those deemed unable to meet specific protocol requirements, such as scheduled visits; (ii) Those deemed unable to administer or tolerate long-term injections as per the patient or the investigator; (iii) Investigator or any sub-investigator, pharmacist, study coordinator, other study staff or relative thereof directly involved in the conduct of the protocol, etc.; (iv) Presence of any other conditions (e.g., geographic, social, etc.) actual or anticipated, that the investigator feels would restrict or limit the patient's participation for the duration of the study.

30. Laboratory findings obtained at during screening period (not including randomization [week 0] labs, unless otherwise noted): (1) Positive test for hepatitis B surface antigen and/or hepatitis C antibody (confirmed by reflexive testing). (2) LDL-C>250 mg/dL (>6.47 mmol/L). (3) TG>400 mg/dL (>4.52 mmol/L) (1 repeat lab is allowed). (4) Positive serum or urine pregnancy test (including week 0) in women of childbearing potential. (5) eGFR<30 mL/min/1.73 m2 according to the 4-variable MDRD Study Equation (calculated by central laboratory) (6) Alanine aminotransferase (ALT) or aspartate aminotransferase (AST)>3× upper limit of normal (ULN) (1 repeat lab is allowed). (7) CPK>3×ULN (1 repeat lab is allowed). (8) TSH<lower limit of normal (LLN) or >ULN.

31. All contraindications to the active comparators (EZE, rosuvastatin) and background therapies or warnings/precautions of use (when appropriate) as displayed in the respective National Product Labeling.

32. Known hypersensitivity to monoclonal antibody therapeutics.

33. Pregnant or breast-feeding women.

34. Women of childbearing potential with no effective contraceptive method of birth control and/or who are unwilling or unable to be tested for pregnancy.

### Study Treatments

Each patient received an SC injection Q2W (mAb316P or placebo-mAb316P) and took 2 oral blinded medications daily (rosuvastatin and EZE or placebo-EZE).

The injectable study treatment was a single SC injection of 1 mL for a 75 mg or 150 mg dose of mAb316P or placebo-mAb316P provided in an auto-injector, administered in the abdomen, thigh, or outer area of the upper arm. The first injection of study drug was administered at the clinical site, as soon as possible after the patient was randomized into the study. The patient was monitored at the clinical site for at least 30 minutes following the first injection. The patient/caregiver administered subsequent injections outside of the clinic, according to the dosing schedule. On days where the clinic study visit coincided with dosing, the dose of study drug were administered after all study assessments had been performed and all laboratory samples collected.

Subcutaneous dosing of study drug was preferably administered Q2W at approximately the same time of day (based upon patient preference); it was acceptable for dosing to fall within a window of +/−3 days.

In the event an injection was delayed by more than 7 days or completely missed, the patient was instructed to return to the original schedule of study drug dosing without administering additional injections. If the delay was less than or equal to 7 days from the missed date the patient was instructed to administer the delayed injection and then resume the original dosing schedule.

Oral study treatments were rosuvastatin and EZE or placebo-EZE. The first dose of oral study drug was administered at the clinical site, as soon as possible after the patient was randomized into the study. The patients continued daily dosing with oral medication through week 22 (day 155). On days where the clinic study visit coincided with dosing, the dose of oral study drug was administered after all study assessments had been performed and all laboratory samples collected.

### Investigational Treatment

Sterile mAb316P drug product was supplied at a concentration of 75 mg/mL or 150 mg/mL in histidine, pH 6.0, polysorbate 20, and sucrose in an auto-injector.

Placebo matching mAb316P was supplied in the same formulation as mAb316P, without the addition of protein, in an auto-injector.

Ezetimibe 10 mg was provided as over-encapsulated tablets. Matching placebo capsules for EZE were supplied.

Rosuvastatin 10 mg, 20 mg, and 40 mg were supplied as matching over-encapsulated tablets.

### Dose Modification (“Up-Titration Option”)

The dose of mAb316P was increased, in a blinded manner, from 75 mg to 150 mg SC Q2W, starting at week 12, based on baseline CV risk in the following circumstances:

A. Patients with heFH or non-FH and a history of documented CHD (defined elsewhere herein), or non-CHD CVD (defined elsewhere herein), or diabetes mellitus with target organ damage: (1) Continue mAb316P 75 mg Q2W, if the week 8 LDL-C is <70 mg/dL (1.8 mmol/L), or (2) Dose up-titrate to mAb316P 150 mg Q2W, if the week 8 LDL-C is ≧70 mg/dL (1.8 mmol/L).

B. Patients with heFH or non-FH, without CHD or non-CHD CVD (defined elsewhere herein), but with a calculated 10-year fatal CVD risk SCORE ≧5%, or with moderate CKD, or with diabetes mellitus but no target organ damage: (1) Continue mAb316P 75 mg Q2W, if the week 8 LDL-C is <100 mg/dL (2.59 mmol/L), or (2) Dose up-titrate to mAb316P 150 mg Q2W, if the week 8 LDL-C is ≧100 mg/dL (2.59 mmol/L).

To maintain the blind, the sites and the sponsor's operational team were blinded to dose modification.

### Concomitant Medications

Concomitant medications were preferably kept to a minimum during the study. If considered necessary for the patients' welfare and unlikely to interfere with study drug, concomitant medications (other than those that are prohibited during the study) were permitted to be given at the discretion of the investigator, with a stable dose (when possible).

Addition of Concomitant Lipid-Modifying Treatment:

During the double-blind treatment period, addition of other LMTs was permitted only under certain conditions: (1) exceptional circumstances—overriding concerns (including, but not limited to, TG alert, below, posted by the central lab) warrant such changes, per the investigator's judgment, or (2) a confirmed TG alert—the patient meets the pre-specified TG alert (TG≧500 mg/dL [5.65 mmol/L). For a TG alert that was confirmed by repeat testing, the investigator was instructed to perform investigations, manage the patient, and add other LMT per his/her medical judgment. For the above circumstances, lab alerts were sent. During the follow-up period, patients were allowed to resume their usual (pre-randomization) statin therapy and addition of other LMTs is permitted.

Permitted Medications:

Nutraceutical products or over-the-counter therapies that may affect lipids were allowed only if they had been used at a stable dose for at least 4 weeks before the screening visit, during the screening period, and maintained during the study. Examples of such nutraceutical products or over-the-counter therapies include omega-3 fatty acids at doses <1000 mg, plant stanols such as found in Benecol, flax seed oil, and psyllium.

Prohibited Medications:

Prohibited concomitant medications from the initial screening visit until the end of the study visit included the following: (1) Statins (other than rosuvastatin provided as blinded study medication), (2) EZE (other than that provided as blinded medication), (3) Fibrates, other than fenofibrate, and (4) Red yeast rice products.

### Study Endpoints

Baseline characteristics included standard demography (e.g., age, race, weight, height, etc.), disease characteristics including medical history, and medication history for each patient.

Primary Efficacy Endpoint:

The primary efficacy endpoint was the percent change in calculated LDL-C from baseline to week 24, which is defined as: 100× (calculated LDL-C value at week 24−calculated LDL-C value at baseline)/calculated LDL-C value at baseline. The baseline calculated LDL-C value was the last LDL-C level obtained before the first double-blind study drug injection. The calculated LDL-C at week 24 was the LDL-C level obtained within the week 24 analysis window and during the main efficacy period. The main efficacy period is defined as the time from the first double-blind study drug injection up to 21 days after the last double-blind study drug injection or up to the upper limit of the week 24 analysis window, whichever comes first.

All calculated LDL-C values (scheduled or unscheduled, fasting or not fasting) were allowed to be used to provide a value for the primary efficacy endpoint if appropriate according to above definition. The analysis window used to allocate a time point to a measurement was defined in a statistical analysis plan (SAP).

Secondary Efficacy Endpoints:

Secondary endpoints of the present study were as follows:

(1) The percent change in calculated LDL-C from baseline to week 12: similar definition and rules as for primary efficacy endpoint, except that the calculated LDL-C at week 12 will be the LDL-C level obtained within the week 12 analysis window and during the 12-week efficacy period.

(2) The percent change in ApoB from baseline to week 24. Same definition and rules as for the primary endpoint.

(3) The percent change in non-HDL-C from baseline to week 24. Same definition and rules as for the primary endpoint.

(4) The percent change in total-C from baseline to week 24. Same definition and rules as for the primary endpoint.

(5) The percent change in ApoB from baseline to week 12. Same definition and rules as for the percent change in calculated LDL-C from baseline to week 12.

(6) The percent change in non-HDL-C from baseline to week 12. Same definition and rules as for the percent change in calculated LDL-C from baseline to week 12.

(7) The percent change in total-C from baseline to week 12. Same definition and rules as for the percent change in calculated LDL-C from baseline to week 12.

(8) The proportion of patients reaching LDL-C goal at week 24, i.e., LDL C<70 mg/dL (1.81 mmol/L) for patients with documented CHD, or non-CHD CVD, or diabetes mellitus with target organ damage, or <100 mg/dL (2.59 mmol/L) for patients with heFH or non-FH, without CHD or non-CHD CVD, but with a calculated 10-year fatal CVD risk SCORE ≧5%, or with moderate CKD, or with diabetes mellitus but no target organ damage, defined as: (number of patients whose calculated LDL-C value at week 24 reach LDL-C goal/number of patients in the (modified intent-to-treat [mITT population])*100, using definition and rules used for the primary endpoint.

(9) The percent change in Lp(a) from baseline to week 24. Same definition and rules as for the primary endpoint.

(10) The percent change in HDL-C from baseline to week 24. Same definition and rules as for the primary endpoint.

(11) The percent change in HDL-C from baseline to week 12. Same definition and rules as for the percent change in calculated LDL-C from baseline to week 12.

(12) The percent change in Lp(a) from baseline to week 12. Same definition and rules as for the percent change in calculated LDL-C from baseline to week 12.

(13) The percent change in fasting TG from baseline to week 24. Same definition and rules as for the primary endpoint.

(14) The percent change in fasting TG from baseline to week 12. Same definition and rules as for the percent change in calculated LDL-C from baseline to week 12.

(15) The percent change in ApoA-1 from baseline to week 24. Same definition and rules as for the primary endpoint.

(16) The percent change in ApoA-1 from baseline to week 12. Same definition and rules as for the percent change in calculated LDL-C from baseline to week 12.

(17) The proportion of patients reaching LDL-C goal at weeks 12, i.e., LDL-C<70 mg/dL (1.81 mmol/L) for patients with documented CHD, or non-CHD CVD, or diabetes mellitus with target organ damage, or <100 mg/dL (2.59 mmol/L) for patients with heFH or non-FH, without CHD or non-CHD CVD, but with a calculated 10-year fatal CVD risk SCORE ≧5%, or with moderate CKD, or with diabetes mellitus but no target organ damage.

(18) The proportion of patients reaching LDL-C<100 mg/dL (2.59 mmol/L) at week 24.

(19) The proportion of patients reaching LDL-C<70 mg/dL (1.81 mmol/L) at week 24.

(20) The proportion of patients reaching LDL-C<100 mg/dL (1.81 mmol/L) at week 12

(21) The proportion of patients reaching LDL-C<70 mg/dL (2.59 mmol/L) at week 12

(22) The absolute change in calculated LDL-C (mg/dL and mmol/L) from baseline to weeks 12 and 24.

(23) The change in ratio ApoB/ApoA-1 from baseline to weeks 12 and 24.

(24) The proportion of patients with ApoB<80 mg/dL (0.8 mmol/L) at weeks 12 and 24.

(25) The proportion of patients with non-HDL-C<100 mg/dL at weeks 12 and 24.

The proportion of patients with calculated LDL-C<70 mg/dL (1.81 mmol/L) and/or ≧50% reduction in calculated LDL-C(if calculated LDL-C≧70 mg/dL [1.81 mmol/L]) at weeks 12 and 24.

Other Endpoints:

(1) Anti-mAB316P anti-drug-antibody status (positive/negative) and titers assessed throughout the study; (2) The percent change in high-sensitivity C-reactive protein (hs-CRP) from baseline to weeks 12 and 24; (3) The absolute change in homeostasis model assessment for insulin resistance (HOMA-IR) (%) from baseline to weeks 12 and 24; and (4) The absolute change in HbA1c (%) from baseline to weeks 12 and 24.

### Study Procedures

Medical/surgical history, medication history, demographics, height, hepatitis B surface antigen, TSH, and serum pregnancy testing were performed for the purpose of determining study eligibility or characterizing the baseline population.

All laboratory samples were collected before the dose of study drug was administered. Blood samples for lipid panels were collected in the morning, in fasting condition (i.e., overnight, at least a 10-hour fast and refrain from smoking) for all clinic visits. Alcohol consumption within 48 hours and intense physical exercise within 24 hours preceding blood sampling were discouraged. Note: if the patient was not in a fasting condition, the blood sample was not collected and a new appointment was scheduled the day after (or as close as possible to this date) with a reminder to be fasted.

Total-C, HDL-C, TG, ApoB, ApoA-1, and Lp(a) were directly measured by a central laboratory. LDL-C was calculated using the Friedewald formula. If TG values exceeded 400 mg/dL (4.52 mmol/L) then the central lab reflexively measured (via the beta quantification method) the LDL-C rather than calculating it. Non-HDL-C was calculated by subtracting HDL-C from the total-C. Ratio ApoB/ApoA-1 was calculated.

Lipid Panel (Fasting):

Blood samples for the lipid panel (total-C, TG, HDL-C, and calculated LDL-C) were collected after at least a 10-hour fast at pre-specified time points.

Specialty Lipid Panel (Fasting):

Blood samples for the specialty lipid panel (ApoB, ApoA-1, ApoB/ApoA-1 ratio, and Lp[a]) were collected after at least a 10-hour fast at pre-specified time points.

Blood Pressure and Heart Rate:

Blood pressure and heart rate were assessed at pre-specified time points. Blood pressure was preferably measured in sitting position under standardized conditions, approximately at the same time of the day, on the same arm, with the same apparatus (after the patient has rested comfortably in sitting position for at least 5 minutes). At the first screening visit, blood pressure was preferably measured in both arms. The arm with the highest diastolic pressure was determined at this visit, and blood pressure was measured on this arm throughout the study. This highest value was recorded in the electronic case report form (eCRF). Heart rate was measured at the time of the measurement of blood pressure.

Physical Examination:

A thorough and complete physical examination, including height and weight, was performed at the baseline visit (visit 3). Physical exam with body weight was performed at pre-specified time points.

Body Weight and Height:

Body weight were obtained with the patient wearing undergarments or very light clothing and no shoes, and with an empty bladder. The same scale was preferably used throughout the study. The use of calibrated balance scales was recommended.

Electrocardiogram:

Electrocardiograms were performed before blood was drawn during visits that required blood draws. A standard 12-lead ECG was performed at pre-specified time points. The 12-lead ECGs were performed after at least 10 minutes rest and in the supine position. The electrodes were positioned at the same place, as much as possible, for each ECG recording throughout the study. The ECG was interpreted locally by the investigator. Each trace was analyzed in comparison with the screening recorded trace.

Laboratory Testing:

All laboratory samples were collected before the dose of study drug was administered. Samples for laboratory testing were collected at pre-specified time points and analyzed by a central laboratory during the study.

### Results

### Subject Disposition

A total of 305 patients were randomized, distributing patients evenly across the treatment arms within each of the rosuvastatin dosing regimens (i.e. 10 mg rosuvastatin regimen: 48-49 patients per treatment arm; and 20 mg rosuvastatin regimen: 47-53 patients per treatment arm). In this study, all 305 randomized patients received study treatment, and therefore the safety population contained 305 patients. Seven patients from the randomized population were excluded from the ITT population due to lack of post-baseline LDL-C assessments. Further, five patients were excluded from the mITT population due to a lack of on-treatment LDL-C assessments.

Combining both baseline rosuvastatin groups together, 87 (84.5%), 84 (83.2%) and 90 (89.1%) patients randomized to the mAb316P add-on, ezetimibe add-on and double-dose rosuvastatin groups, respectively, completed 24 weeks of the double-blind treatment period (defined as at least 22 weeks of treatment and Week 24 visit performed). Baseline patient characteristics were generally similar across treatment groups as summarized in Tables 24A and 24B.

TABLE 24A
Baseline Characteristics (Entry Statin = Rosuvastatin 10 mg [n = 145])

| | mAb316P 75/150 | | EZE 10 mg + RSV 10 | | | |
|---|---|---|---|---|---|---|
| | mg + RSV 10 mg (n = 49) | | mg (n = 48) | | RSV 20 mg (n = 48) | |
| Age, years, mean (SD) | 62.2 | (11.1) | 60.4 | (10.4) | 61.5 | (11.1) |
| Male % (n) | 63.3 | (31) | 54.2 | (26) | 68.8 | (33) |
| Race, White % (n) | 91.8 | (45) | 87.5 | (42) | 77.1 | (37) |
| Ethnicity, | 7 | (14.3) | 6 | (12.5) | 6 | (12.5) |
| Hispanic/Latino n (%) | | | | | | |
| BMI, kg/m², mean (SD) | 31.8 | (7.7) | 32.1 | (7.3) | 32.0 | (6.2) |
| HeFH n (%) | 8 | (16.3) | 6 | (12.5) | 4 | (8.3) |
| CHD history, n (%) | 23 | (46.9) | 29 | (60.4) | 25 | (52.1) |
| CHD risk equivalent, n (%) | 16 | (32.7) | 12 | (25.0) | 15 | (31.3) |
| Hypertension n (%) | 36 | (73.5) | 33 | (68.8) | 34 | (70.8) |
| Type 2 diabetes n (%) | 19 | (38.8) | 23 | (47.9) | 28 | (58.3) |
| Use of lipid-lowering | 11 | (22.4) | 8 | (16.7) | 11 | (22.9) |
| therapy other than | | | | | | |
| statin, n (%) | | | | | | |
| LDL-C (calculated) | 107.3 | (26.4) | 102.4 | (41.9) | 105.9 | (36.0) |
| mean (SD), mg/dL | | | | | | |
| Non-HDL-C, mean | 138.0 | (37.5) | 131.7 | (48.8) | 136.9 | (41.7) |
| (SD) | | | | | | |
| Apo B, mean (SD) | 93.4 | (22.6) | 89.0 | (25.9) | 92.7 | (25.2) |
| Lp(a), median (Q1:Q3) | 22.0 | (8.0:74.0) | 38.5 | (14.0:106.0) | 26.0 | (8.0:48.0) |
| Fasting Triglycerides, | 116.0 | (86.0:199.0) | 127.0 | (95.0:163.5) | 130.5 | (90.5:203.0) |
| median (Q1:Q3) | | | | | | |
| HDL-C, mean (SD) | 49.4 | (12.7) | 51.0 | (13.0) | 48.5 | (14.2) |

TABLE 24B
Baseline Characteristics (Entry Statin = Rosuvastatin 20 mg [n = 160])

| | mAb316P 75/150 | | EZE 10 mg + RSV 20 | | | |
|---|---|---|---|---|---|---|
| | mg + RSV 20 mg (n = 54) | | mg (n = 53) | | RSV 40 mg (n = 53) | |
| Age, years, mean (SD) | 57.9 | (8.9) | 63.1 | (10.2) | 60.6 | (10.1) |
| Male % (n) | 51.9 | (28) | 58.5 | (31) | 71.7 | (38) |
| Race, White % (n) | 77.8 | (42) | 86.8 | (46) | 83.0 | (44) |
| Ethnicity, | 5 | (9.3) | 7 | (13.2) | 10 | (18.9) |
| Hispanic/Latino n (%) | | | | | | |
| BMI, kg/m², mean (SD) | 30.2 | (6.0) | 30.2 | (5.4) | 31.5 | (6. 7) |
| HeFH n (%) | 6 | (11.1) | 8 | (15.1) | 9 | (17.0) |
| CHD history, n (%) | 32 | (59.3) | 32 | (60.4) | 36 | (67.9) |
| CHD risk equivalent, n (%) | 11 | (20.4) | 11 | (20.8) | 14 | (26.4) |
| Hypertension n (%) | 40 | (74.1) | 36 | (67.9) | 42 | (79.2) |
| Type 2 diabetes n (%) | 18 | (33.3) | 21 | (39.6) | 17 | (32.1) |
| Use of lipid-lowering | 11 | (20.4) | 13 | (24.5) | 9 | (17.0) |
| therapy other than | | | | | | |
| statin, n (%) | | | | | | |
| LDL-C (calculated) | 118.3 | (32.2) | 119.0 | (48.0) | 112.9 | (43.3) |
| mean (SD), mg/dL | | | | | | |
| Non-HDL-C, mean | 145.8 | (36.6) | 149.0 | (49.7) | 143.6 | (44.4) |
| (SD) | | | | | | |
| Apo B, mean (SD) | 97.8 | (20.4) | 100.8 | (25.9) | 96.7 | (23.6) |
| Lp(a), median (Q1:Q3) | 49.5 | (16.0:105.0) | 35.5 | (15.0:76.0) | 28.0 | (6.0:70.0) |
| Fasting Triglycerides, | 116.0 | (91.0:179.0) | 143.0 | (93.0:192.0) | 143.0 | (108.0:190.0) |
| median (Q1:Q3) | | | | | | |
| HDL-C, mean (SD) | 51.8 | (11.0) | 52.2 | (13.7) | 46.9 | (13.6) |

Treatment compliance with study drug injections was high in all treatment groups within the pooled dose regimen, with a mean overall compliance of 97.5% in the mAb316P add-on group, 98.6% in the ezetimibe add-on group and 98.7% in the double-dose rosuvastatin group. For the most part, these completer patients were evenly distributed among the study treatment groups. 60 (19.7%) patients discontinued the study treatment early. The individual treatment arm patient discontinuation rates are: 5 (10.4%) in the rosuvastatin 20 mg arm, 14 (29.2%) in the rosuvastatin 10 mg+EZE arm; 11 (22.4%) in the rosuvastatin 10 mg+mAb316P arm, 8 (15.1%) in the rosuvastatin 40 mg arm, 9 (17.0%) in the rosuvastatin 20 mg+EZE arm, and 13 (24.1%) in the rosuvastatin 20 mg+mAb316P arm.

### Efficacy Results

Overall, demographic characteristics, baseline disease characteristics, baseline efficacy lipid parameters, LMT history and background LMT use were comparable among the treatment arms. Particularly, the mean baseline LDL-C values confirmed homogeneity at baseline with individual treatment arm means ranging from 102.4 mg/dL to 107.3 mg/dL in the 10 mg rosuvastatin regimen; and from 112.9 mg/dL to 119.0 mg/dL in the 20 mg rosuvastatin regimen.

The study included 2 rosuvastatin dose regimens and 6 arms (described in the Study Design section above). The four primary efficacy pairwise comparisons are defined within each rosuvastatin regimen, as randomized in the IVRS/IWRS (i.e., 2 pairwise comparisons within each rosuvastatin regimen) (See Table 25).

TABLE 25
Primary Pairwise Comparisons

| Rosuvastatin | |
|---|---|
| Dose Regimen | Pairwise Comparison |
| 10 mg regimen | Comparison 1: mAb316P + rosuvastatin 10 mg arm vs. |
| | rosuvastatin 20 mg arm |
| | Comparison 2: mAb316P + rosuvastatin 10 mg arm vs. |
| | EZE + rosuvastatin 10 mg arm |
| 20 mg regimen | Comparison 3: mAb316P + rosuvastatin 20 mg arm vs. |
| | rosuvastatin 40 mg arm |
| | Comparison 4: mAb316P + rosuvastatin 20 mg arm vs. |
| | EZE + rosuvastatin 20 mg arm |

To account for statistical testing of the four primary pairwise treatment comparisons described above, the alpha level is adjusted for multiplicity to 0.0125 for each comparison hereby controlling for the overall study alpha level.

The primary and key secondary efficacy analysis results are set forth in Tables 23 through 40. For clarification, the ITT analysis is defined for patients in the ITT population and includes all endpoint assessments in an analysis window, regardless of study treatment dosing status (i.e. includes post-treatment assessments). The on-treatment analysis is defined for patients in the mITT population and includes all endpoint assessments from the first double-blind study drug (capsule or injection, whichever comes first) up to 21 days after the last double-blind study drug injection or 3 days after the last capsule intake, whichever comes first (i.e. includes assessments in the efficacy treatment period). NOTE: P-values that are considered statistically significant as described in the hierarchical testing order at the 0.0125 level are followed by an * in the Tables 26 through 43.

TABLE 26
The primary efficacy analysis for percent change from baseline
of calculated LDL-C to week 24 in the ITT Population

| | Comparison | Comparison | Comparison | Comparison |
|---|---|---|---|---|
| | 1 | 2 | 3 | 4 |
| LS Mean | −34.2% | −36.1% | −20.3% | −25.3% |
| Difference | | | | |
| P-value | <.0001* | <.0001* | 0.0453 | 0.0136 |

TABLE 27
The key secondary efficacy analyses for percent change from baseline
of calculated LDL-C to week 24 in the mITT Population

| | Comparison | Comparison | Comparison | Comparison |
|---|---|---|---|---|
| | 1 | 2 | 3 | 4 |
| LS Mean | −35.2% | −33.2% | −24.5% | −24.9% |
| Difference | | | | |
| P-value | <.0001* | <.0001* | 0.0131 | 0.0115 |

TABLE 28
The key secondary efficacy analyses for percent change from
baseline of calculated LDL-C to week 12 in the ITT Population

| | Comparison | Comparison | Comparison | Comparison |
|---|---|---|---|---|
| | 1 | 2 | 3 | 4 |
| LS Mean | −32.5% | −32.2% | −10.2% | −12.9% |
| Difference | | | | |
| P-value | <.0001* | <.0001* | 0.1747 | 0.0861 |

TABLE 29
The key secondary efficacy analyses for percent change from baseline
of calculated LDL-C to week 12 in the mITT Population

| | Comparison | Comparison | Comparison | Comparison |
|---|---|---|---|---|
| | 1 | 2 | 3 | 4 |
| LS Mean | −35.3% | −32.3% | −12.3% | −13.3% |
| Difference | | | | |
| P-value | <.0001* | <.0001* | 0.0980 | 0.0718 |

TABLE 30
The key secondary efficacy analyses for percent change
from baseline of Apo B to week 24 in the ITT Population

| | Comparison | Comparison | Comparison | Comparison |
|---|---|---|---|---|
| | 1 | 2 | 3 | 4 |
| LS Mean | −29.2% | −26.8% | −18.5% | −17.1% |
| Difference | | | | |
| P-value | <.0001* | <.0001* | 0.0024 | 0.0057 |

TABLE 31
The key secondary efficacy analyses for percent change from
baseline of Apo B to week 24 in the mITT Population

| | Comparison | Comparison | Comparison | Comparison |
|---|---|---|---|---|
| | 1 | 2 | 3 | 4 |
| LS Mean | −30.7% | −28.3% | −17.7% | −17.8% |
| Difference | | | | |
| P-value | <.0001* | <.0001* | 0.0027 | 0.0027 |

TABLE 32
The key secondary efficacy analyses for percent change from
baseline of non-HDL-C to week 24 in the ITT Population

| | Comparison | Comparison | Comparison | Comparison |
|---|---|---|---|---|
| | 1 | 2 | 3 | 4 |
| LS Mean | −31.4% | −29.3% | −20.1% | −18.4% |
| Difference | | | | |
| P-value | <.0001* | <.0001* | 0.0063 | 0.0133 |

TABLE 33
The key secondary efficacy analyses for percent change from
baseline of non-HDL-C to week 24 in the mITT Population

| | Comparison | Comparison | Comparison | Comparison |
|---|---|---|---|---|
| | 1 | 2 | 3 | 4 |
| LS Mean | −32.8% | −28.2% | −20.7% | −17.4% |
| Difference | | | | |
| P-value | <.0001* | <.0001* | 0.0008 | 0.0046 |

TABLE 34
The key secondary efficacy analyses for percent change from
baseline of Total-C to week 24 in the ITT Population

| | Comparison | Comparison | Comparison | Comparison |
|---|---|---|---|---|
| | 1 | 2 | 3 | 4 |
| LS Mean | −20.6% | −20.3% | −12.1% | −8.2% |
| Difference | | | | |
| P-value | <.0001* | <.0001* | 0.0193 | 0.1134 |

TABLE 35
The key secondary efficacy analyses for percent change
from baseline of Apo-B to week 12 in the ITT Population

| | Comparison | Comparison | Comparison | Comparison |
|---|---|---|---|---|
| | 1 | 2 | 3 | 4 |
| LS Mean | −28.1% | −24.0% | −15.3% | −14.7% |
| Difference | | | | |
| P-value | <.0001* | <.0001* | 0.0013 | 0.0022 |

TABLE 36
The key secondary efficacy analyses for percent change from
baseline of Non-HDL-C to week 12 in the ITT Population

| | Comparison | Comparison | Comparison | Comparison |
|---|---|---|---|---|
| | 1 | 2 | 3 | 4 |
| LS Mean | −29.5% | −24.9% | −11.8% | −11.0% |
| Difference | | | | |
| P-value | <.0001* | <.0001* | 0.0226 | 0.0342 |

TABLE 37
The key secondary efficacy analyses for percent change from
baseline of Total-C to week 12 in the ITT Population

| | Comparison | Comparison | Comparison | Comparison |
|---|---|---|---|---|
| | 1 | 2 | 3 | 4 |
| LS Mean | −20.1% | −17.2% | −5.5% | −5.5% |
| Difference | | | | |
| P-value | <.0001* | <.0001* | 0.1563 | 0.1629 |

TABLE 38
The key secondary efficacy analyses for proportion of
very high CV risk patients reaching calculated LDL-C <
70 mg/dL or high CV risk patients reaching calculated
LDL-C < 100 mg/dL at Week 24 in the ITT Population

| | Comparison | Comparison | Comparison | Comparison |
|---|---|---|---|---|
| | 1 | 2 | 3 | 4 |
| Odds Ratio | 10.2 | 7.8 | 4.7 | 2.3 |
| P-value | 0.0001* | 0.0007* | 0.0019 | 0.0759 |

TABLE 39
The key secondary efficacy analyses for proportion of
very high CV risk patients reaching calculated LDL-C <
70 mg/dL or high CV risk patients reaching calculated
LDL-C < 100 mg/dL at Week 24 in the mITT Population

| | Comparison | Comparison | Comparison | Comparison |
|---|---|---|---|---|
| | 1 | 2 | 3 | 4 |
| Odds Ratio | 12.4 | 8.9 | 5.9 | 2.7 |
| P-value | 0.0001* | 0.0009* | 0.0012 | 0.0602 |

TABLE 40
The key secondary efficacy analyses for proportion
of patients reaching calculated LDL-C < 70 mg/dL
(1.81 mmol/L) at Week 24 in the ITT Population

| | Comparison | Comparison | Comparison | Comparison |
|---|---|---|---|---|
| | 1 | 2 | 3 | 4 |
| Odds Ratio | 18.6 | 11.6 | 6.1 | 2.5 |
| P-value | <.0001* | <.0001* | 0.0006 | 0.0657 |

TABLE 41
The key secondary efficacy analyses for proportion
of patients reaching calculated LDL-C < 70 mg/dL
(1.81 mmol/L) at Week 24 in the mITT Population

| | Comparison | Comparison | Comparison | Comparison |
|---|---|---|---|---|
| | 1 | 2 | 3 | 4 |
| Odds Ratio | 20.3 | 12.7 | 9.0 | 3.4 |
| P-value | <.0001* | 0.0002 | 0.0002 | 0.0255 |

TABLE 42
The key secondary efficacy analyses for percent change
from baseline of Lp(a) to week 24 in the ITT Population

| | Comparison | Comparison | Comparison | Comparison |
|---|---|---|---|---|
| | 1 | 2 | 3 | 4 |
| LS Mean | −23.9% | −23.6% | −17.5% | −16.9% |
| Difference | | | | |
| P-value | <.0001* | <.0001* | 0.0123 | 0.0131 |

TABLE 43
The statistical hierarchical testing for the efficacy endpoints
terminated at the percent change from baseline of HDL-C at week
24 in the ITT population with a lack of statistical significance

| | Comparison | Comparison | Comparison | Comparison |
|---|---|---|---|---|
| | 1 | 2 | 3 | 4 |
| LS Mean | 7.4% | 5.1% | 5.7% | 9.0% |
| Difference | | | | |
| P-value | 0.0311 | 0.1491 | 0.0866 | 0.0072 |

LDL-C results at week 24 are further summarized in Table 44.

TABLE 44
Effect of mAb316P on LDL-C at Week 24

| | Baseline Rosuvastatin 10 mg | | | Baseline Rosuvastatin 20 mg | | |
|---|---|---|---|---|---|---|
| | | EZE + | mAb316P + | | EZE + | mAb316P + |
| Randomized | RSV 20 | RSV 10 | RSV 10 | RSV 40 | RSV 20 | RSV 20 |
| pts | N = 48 | N = 48 | N = 49 | N = 53 | N = 53 | N = 54 |
| ITT population | 48 | 47 | 48 | 52 | 50 | 53 |
| (n) | | | | | | |
| Baseline (ITT), | 105.9 | 102.0 | 107.8 | 113.7 | 119.4 | 118.1 |
| mean (SD), | (36.0) | (42.3) | (26.5) | (43.3) | (48.5) | (32.5) |
| mg/dL | | | | | | |
| W24 % change | −16.3 | −14.4 | −50.6 | −15.9 | −11.0 | −36.3 |
| from baseline, | (4.1) | (4.4) | (4.2) | (7.1) | (7.2) | (7.1) |
| LS mean (SE) | | | | | | |
| Difference | −34.2 | −36.1 | | −20.3 | −25.3 | |
| mAb316P vs | (5.9) | (6.1) | | (10.1) | (10.1) | |
| comparator | | | | | | |
| p-value | <.0001* | <.0001* | | 0.0453 | 0.0136 | |
| mAb316P dose | | | 15.9 | | | 20.8 |
| up-titration | | | | | | |
| from 75 to 150 | | | | | | |
| mg Q2W at | | | | | | |
| W12, % pts | | | | | | |

*Level of statistical significance: 0.0125 following Bonferroni adjustment for multiplicity.
^{†}Very high-risk: <70 mg/dL; high-risk: <100 mg/dL.

The proportion of patients reaching LDL-C goal (i.e., a calculated LDL-C level of less than 70 mg/dL for very high risk CV patients, or a calculated LDL-C level of less than 100 mg/dL for high risk patients) at week 24 is summarized in Table 45.

TABLE 45
Combined Estimate for Proportion of Patients Reaching LDL-C Goal (%)

| | Baseline Rosuvastatin 10 mg | | | Baseline Rosuvastatin 20 mg | | |
|---|---|---|---|---|---|---|
| | | EZE + | mAb316P + | | EZE + | mAb316P + |
| Randomized | RSV 20 | RSV 10 | RSV 10 | RSV 40 | RSV 20 | RSV 20 |
| pts | n = 48 | n = 48 | N = 49 | N = 53 | N = 53 | N = 54 |
| Proportion of | 45.0 | 57.2 | 84.9 | 40.1 | 52.2 | 66.7 |
| Patients | | | | | | |
| Reaching | | | | | | |
| LDL-C goal (%) | | | | | | |
| p-value | 0.0001 | 0.0007 | | 0.0019 | 0.0759 | |
| (mAb316P vs | | | | | | |
| comparator) | | | | | | |

The change in LDL-C levels in the various treatment groups over time is summarized in Table 46.

TABLE 46
Calculated LDL-C Over Time

| | | | Calculated LDL-C LS mean (SE) | | |
|---|---|---|---|---|---|
| Time | Baseline | | Value | Change from | % Change from |
| Point | Rosuvastatin | Treatment | (mg/dL) | Baseline | Baseline |
| Baseline | 10 mg | RSV 20 | 105.9 (5.2) | NA | NA |
| Week 4 | | | 89.1 (3.6) | −16.2 (3.6) | −13.1 (3.2) |
| Week 8 | | | 85.4 (4.3) | −19.9 (4.3) | −17.0 (3.8) |
| Week 12 | | | 85.7 (4.1) | −19.6 (4.1) | −17.1 (4.1) |
| Week 16 | | | 83.6 (4.7) | −21.6 (4.7) | −19.3 (4.8) |
| Week 24 | | | 85.4 (4.3) | −19.9 (4.3) | −16.3 (4.1) |
| Baseline | 10 mg | EZE + | 102.0 (6.2) | NA | NA |
| Week 4 | | RSV 10 | 75.4 (3.7) | −29.9 (3.7) | −27.4 (3.3) |
| Week 8 | | | 81.5 (4.3) | −23.7 (4.3) | −22.4 (3.8) |
| Week 12 | | | 85.7 (4.2) | −19.6 (4.2) | −17.4 (4.2) |
| Week 16 | | | 91.9 (4.8) | −13.4 (4.8) | −10.3 (4.8) |
| Week 24 | | | 87.9 (4.5) | −17.4 (4.5) | −14.4 (4.4) |
| Baseline | 10 mg | mAb316P + | 107.8 (3.8) | NA | NA |
| Week 4 | | RSV 10 | 49.9 (3.6) | −55.3 (3.6) | −53.1 (3.2) |
| Week 8 | | | 49.4 (4.3) | −55.9 (4.3) | −53.8 (3.8) |
| Week 12 | | | 53.9 (4.1) | −51.3 (4.1) | −49.6 (4.1) |
| Week 16 | | | 49.2 (4.7) | −56.1 (4.7) | −54.1 (4.7) |
| Week 24 | | | 53.0 (4.3) | −52.2 (4.3) | −50.6 (4.2) |
| Baseline | 20 mg | RSV 40 | 113.7 (6.0) | NA | NA |
| Week 4 | | | 92.0 (3.4) | −25.1 (3.4) | −20.0 (5.3) |
| Week 8 | | | 91.9 (4.3) | −25.2 (4.3) | −21.2 (5.7) |
| Week 12 | | | 90.4 (4.3) | −26.6 (4.3) | −22.1 (5.3) |
| Week 16 | | | 93.8 (4.8) | −23.3 (4.8) | −18.5 (6.3) |
| Week 24 | | | 95.5 (5.7) | −21.5 (5.7) | −15.9 (7.1) |
| Baseline | 20 mg | EZE + RSV | 119.4 (6.9) | NA | NA |
| Week 4 | | 20 | 79.7 (3.5) | −37.3 (3.5) | −22.1 (5.3) |
| Week 8 | | | 82.0 (4.4) | −35.1 (4.4) | −18.8 (5.8) |
| Week 12 | | | 83.0 (4.3) | −34.0 (4.3) | −19.3 (5.4) |
| Week 16 | | | 84.2 (4.8) | −32.8 (4.8) | −17.7 (6.3) |
| Week 24 | | | 91.6 (5.7) | −25.4 (5.7) | −11.0 (7.2) |
| Baseline | 20 mg | mAb316P + | 118.1 (4.5) | NA | NA |
| Week 4 | | RSV 20 | 69.9 (3.4) | −47.1 (3.4) | −39.6 (5.2) |
| Week 8 | | | 67.4 (4.3) | −49.6 (4.3) | −40.6 (5.7) |
| Week 12 | | | 76.1 (4.2) | −41.0 (4.2) | −32.3 (5.2) |
| Week 16 | | | 64.5 (4.8) | −52.6 (4.8) | −44.8 (6.2) |
| Week 24 | | | 73.5 (5.8) | −43.5 (5.8) | −36.3 (7.1) |

Of the 92 patients in the pooled mAb316P group who received at least one injection of study drug, 17 (18.5%) patients had their dose increased to mAb316P 150 mg Q2W at Week 12; 7 (15.9%) patients in the baseline rosuvastatin 10 mg regimen and 10 (20.8%) patients in the baseline rosuvastatin 20 mg regimen.

In the baseline rosuvastatin 10 mg regimen, mAb316P add-on treatment significantly reduced LDL-C levels at Week 24 versus the other comparators (p<0.0001). LDL-C reductions in the mAb316P add-on group were observed by Week 4 and were maintained through Week 24.

In the baseline rosuvastatin 20 mg regimen, mean reductions from baseline in LDL-C at Week 24 were numerically greater in the mAb316P add-on group versus the other comparators. Due to the statistical test, a p-value of 0.0125 was required to adjust for multiple comparisons, and the mean differences when comparing the mAb316P add-on group with the ezetimibe add-on (difference of −25.3%; 98.75% CI [−50.9 to 0.3]; p=0.0136) and rosuvastatin 40 mg groups (difference of −20.3%; 98.75% CI [−45.8 to 5.1]; p=0.0453) did not reach statistical significance. Measured at Week 4, and through Week 24, the mAb316P add-on group demonstrated greater numeric mean LDL-C reductions from baseline over time compared with the ezetimibe add-on and rosuvastatin 40 mg treatment groups.

In both baseline rosuvastatin regimen groups, LDL-C reductions in the primary analysis were in agreement with the on-treatment results as well as the results of measured LDL-C using beta quantification and the pattern mixture model analysis.

In the baseline rosuvastatin 10 mg regimen groups, the proportion of patients at very high and high CV risk who reached a LDL-C level of <70 mg/dL (1.81 mmol/L) or <100 mg/dL (2.59 mmol/L) at Week 24, respectively, was significantly greater in the mAb316P add-on group (84.9%) compared with the ezetimibe add-on group (57.2%; p=0.0007) and the rosuvastatin 20 mg group (45.0%; p<0.0001). The proportion of patients who reached the more stringent LDL-C level of <70 mg/dL (1.81 mmol/L) at Week 24 was also significantly greater in the mAb316P add-on group (77.8%) compared with the ezetimibe add-on and rosuvastatin 20 mg groups (43.1%; p<0.0001 and 31.3%; p<0.0001), respectively.

In the baseline rosuvastatin 20 mg regimen groups, the proportion of very high and high risk patients who reached a LDL-C level of <70 mg/dL (1.81 mmol/L) or <100 mg/dL (2.59 mmol/L) at Week 24, depending on risk status, was numerically greater in the mAb316P add-on group (66.7%) compared with the ezetimibe add-on group (52.2%; nominal p=0.1177) and rosuvastatin 40 mg treatment group (40.1%; nominal p=0.0022) (FIG. 2). The proportion of patients who reached a LDL-C level of <70 mg/dL (1.81 mmol/L) at Week 24 was also greater in the mAb316P add-on group (60.1%) compared with the ezetimibe add-on group (43.6%; nominal p=0.0657) and rosuvastatin 40 mg group (29.9%; nominal p=0.0006).

Significant reductions in Apo B, non-HDL-C and Lp(a) were seen in the mAb316P add-on group versus other comparators in the baseline rosuvastatin 10 mg regimen. The mAb316P add-on group also produced modest reductions in triglycerides and increases in HDL-C.

In the baseline rosuvastatin 20 mg regimen, numeric decreases in Apo B, non-HDL-C and Lp(a) were observed in the mAb316P add-on group when compared with the ezetimibe add-on and rosuvastatin 40 mg groups.

In summary, the efficacy results from this Example demonstrate that the addition of a PCSK9 antagonist (e.g., mAb316P) to moderate dose statin therapy (e.g., rosuvastatin 10 mg or 20 mg daily) produced greater and more pronounced lipid lowering efficacy than treatment alternatives such as: (a) increasing the patient's daily statin dose (e.g., increasing daily rosuvastatin from 10 mg to 20 mg or from 20 mg to 40 mg); or (b) adding Ezetimibe to the patient's existing moderate dose statin therapy.

### Safety Results

A summary of safety results are presented by the treatment groups of pooled rosuvastatin dose regimens, with the intent to maximize efforts to detect potential safety signals. For the pooled data, three treatment groups were formed by combining treatment arms regardless of the rosuvastatin doses as follows:

(1) The double-dose rosuvastatin treatment group: pooling rosuvastatin 20 mg and rosuvastatin 40 mg;

(2) EZE treatment group: pooling EZE+10 mg+rosuvastatin 10 mg and EZE 10 mg+rosuvastatin 20 mg;

(3) mAb316P treatment group: pooling mAb316P+rosuvastatin 10 mg and mAb316P+rosuvastatin 20 mg.

A total of 305 patients were randomized and received at least a partial dose of study treatment (Safety Population). A high-level safety summary of adverse events and events of interest is as follows:

(1) Treatment-emergent SAEs occurred in 8 (7.9%) patients in the double-dose rosuvastatin treatment group, 8 (7.9%) patients in the EZE treatment group, and 6 (5.8%) patients in the mAb316P treatment group.

(2) One death was reported in this study. Specifically, a 71-year old male patient receiving rosuvastatin 20 mg+EZE study treatment experienced a fatal subdural hematoma on study day 56.

(3) A total of 18 patients discontinued study treatment early due to a treatment emergent adverse event, specifically 5 (5.0%) patients in the double-dose rosuvastatin treatment groups, 8 (7.9%) patients in the pooled EZE treatment group, and 5 (4.9%) patients in the pooled mAb316P treatment group.

(4) TEAEs occurred in 68 (67.3%) patients in the double dose rosuvastatin treatment group, 54 (53.5%) patients in the EZE treatment group, and 58 (56.3%) patients in the mAb316P treatment group. The SOCs with a higher frequency in the mAb316P group as compared to either the double-dose rosuvastatin and EZE treatment groups were: (a) “General disorders and administration site conditions” occurred in 15 (14.6%) patients in the mAb316P treatment group, vs. 10 (9.9%) in the double-dose rosuvastatin treatment group and 8 (7.9%) patient in the EZE treatment group. Particularly, injection site reactions were the most frequently reported TEAE in the mAb316P patients in this SOC and were reported in 4 (3.9%) patients in the mAb316P treatment group, vs. 2 (2.0%) in the double-dose rosuvastatin treatment group and 0 patients in the EZE treatment group. Pyrexia was reported in 2 (1.9%) patients in mAb316P treatment groups vs. 0 patients in the other two groups. (b) “Respiratory, thoracic and mediastinal disorders” occurred in 7 (6.8%) patients in the mAb316P treatment group, vs. 5 (5.0%) patients in the double-dose rosuvastatin treatment group and 2 (2.0%) patients in the EZE treatment group. Only 2 patients in this SOC were reported in more than a single patient: Specifically, nasal congestion in 2 (1.9%) patients in the mAb316P treatment group vs 0 patients in the other two groups. Also, cough was reported in 2 (1.9%) patients in the mAb316P treatment group vs 3 (3.0%) in the double-dose rosuvastatin treatment group and 0 patients in the EZE treatment group. (c) “Skin and subcutaneous tissue disorders” occurred in 8 (7.8%) patients in the mAb316P treatment group, vs. 6 (5.9%) patients in the double-dose rosuvastatin treatment group and 5 (5.0%) patients in the EZE treatment group. Rash occurred in 2 (1.9%) patients in the mAb316P treatment group vs. zero patients in the other two groups. (d) “Psychiatric disorders” occurred in 5 (4.9%) patients in the mAb316P treatment group vs. 3 (3.0%) in the double-dose rosuvastatin treatment group and 4 (4.0%) patients in the EZE treatment group. Insomnia occurred in 2 (1.9%) patients in the mAb316P treatment group vs. zero patients in the other two groups. (e) “Immune system disorders” occurred in 1 (1.0%) patient in the mAb316P treatment group vs. zero patients in the other two groups. The only TEAE in this SOC was a single report of hypersensitivity.

The SOCs with a higher frequency in either the double-dose rosuvastatin and pooled EZE treatment groups compared to the pooled mAb316P treatment group included: infections and infestations, neoplasms benign, malignant and unspecified (incl cysts and polyps), blood and lymphatic system disorders, metabolism and nutrition disorders, nervous system disorders, ear and labyrinth disorders, cardiac disorders, vascular disorders, musculoskeletal and connective tissue disorders, investigations, and injury, poisoning and procedural complications.

The most frequent TEAEs (reported in at least two patients in the mAb316P group) were: upper respiratory tract infection (6), influenza (4), nasopharyngitis (4), urinary tract infection (4), arthralgia (4), myalgia (4), injection site reaction (4), dizziness (3), nausea (3), accidental overdose (3), laceration (3), fatigue (3), sciatica (2), cough (2), nasal congestion (2), diarrhea (2), constipation (2), rash (2), pain in extremity (2), hypokalaemia (2) local swelling (2), pyrexia (2), and insomnia (2).

For TEAEs of special interest (AESIs), results are presented by pre-defined SMQ preferred term groupings:

(1) Treatment-emergent injection site reactions (ISRs) occurred in 2 (2.0%) patients in the double-dose rosuvastatin treatment group, 0 patients in the EZE treatment group, and 4 (3.9%) patients in the mAb316P treatment group. Of the 4 ISRs reported in the alircoumab treatment group, 3 were mild and 1 was moderate in severity. No severe ISRs were reported.

(2) General Allergic TEAEs occurred in 7 (6.9%) patients in the double-dose rosuvastatin treatment group, 2 (2.0%) patients in the EZE treatment group, and 11 (10.7%) patients in the mAb316P treatment group. Two PTs were reported in more than a single patient in the mAb316P treatment group, injection site reaction and rash were both reported in 2 patients. All other general allergic TEAEs were reported in a single patient.

(3) Treatment-emergent neurologic disorders occurred in 2 (2.0%) patients in the double-dose rosuvastatin treatment group, 3 (3.0%) patients in the EZE treatment group, and 2 (1.9%) patients in the mAb316P treatment group.

(4) Treatment-emergent neurocognitive disorders occurred in 2 (2.0%) patients in the double-dose rosuvastatin treatment group, in 1 (1.0%) patients in the EZE treatment group, and in 1 (1.0%) patients in the mAb316P treatment group.

(5) Treatment-emergent cardiovascular events adjudicated positively were collected from 1 (1.0%) patient in the double-dose rosuvastatin treatment group with the adjudicated term “nom-fatal ischemic stroke”, and in the 1 (1.0%) patient in the EZE treatment group with adjudicated terms “non-fatal MI” and “ischemic driven coronary revascularization procedure”. Zero patients in the mAb316P treatment group had any CV event adjudicated positive.

Lastly, 13 (12.6%) patients reported 2 consecutive calculated LDL-C measurements below 25 mg/dL and all occurrences were reported from the mAb316P treatment group. Five of the 13 patients had TEAEs which occurred the day on or after the first of the two consecutive low LDL-C values. None of these TEAE was serious or led to treatment discontinuations. Each of the preferred terms occurred in only one patient: laryngitis, restless legs syndrome, cough, dyspepsia, dermatitis contact, muscle tightness, plantar fasciitis, pruritus genital, fatigue, local swelling, wrist fracture and laceration.

### Conclusions

In the baseline rosuvastatin 10 mg regimen, treatment with add-on mAb316P therapy significantly reduced calculated LDL-C levels at Week 24 when compared with doubling the dose of rosuvastatin regimen or ezetimibe add-on therapy. In the rosuvastatin 10 mg baseline regimen group, mAb316P add-on therapy also significantly improved most key secondary endpoints, Apo B, non-HDL-C and Lp(a), including the proportion of patients achieving pre-specified LDL-C levels.

Numerically greater changes in calculated LDL-C levels were observed in the group receiving add-on mAb316P to rosuvastatin 20 mg when compared with doubling the rosuvastatin dose or an ezetimibe add-on treatment regimen, but due to our study's statistical test where a p-value of 0.0125 was required to adjust for multiple comparisons, they were not statistically significant. However, the effect of mAb316P in the baseline rosuvastatin 20 mg treatment regimen is directionally consistent with the results in the baseline rosuvastatin 10 mg treatment regimen. In addition, while the magnitude of the effect in the add-on mAb316P rosuvastatin 20 mg group is smaller than in the add-on mAb316P rosuvastatin 10 mg group, the standard error is approximately twice as large (7.1 versus 4.2, respectively) and is greater than was estimated in the original power calculations. This may explain a portion of the lack of statistical significance in the baseline rosuvastatin 20 mg treatment regimen.

This study also evaluated a flexible mAb316P dosing regimen that allowed for the mAb316P dose to be increased only when patients did not reach their individual target LDL-C level (<70 mg/dL or <100 mg/dL in very-high and high risk patients, respectively) by a pre-specified time point (Week 8 in our study). All patients in the mAb316P groups began the study on an mAb316P regimen of 75 mg Q2W and more than 80% of patients were maintained on the 75 mg Q2W dose; 17 patients (18.5%) had their dose increased to mAb316P 150 mg Q2W at Week 12 in a blinded manner. Patients who did not have their dose increased maintained the reduction in calculated LDL-C levels observed at Week 12 to Week 24, while patients who had their dose increased at the Week 12 visit showed a further reduction in LDL-C levels from Week 12 to Week 24.

Overall, in this ≧24-week study, mAb316P was well tolerated and the numbers of patients reporting TEAEs were similar across treatment groups, suggesting durability of effect. No safety signals were detected for mAb316P add-on therapy when compared with doubling the statin dose or ezetimibe add-on therapy. The overall rate of AEs of special interest was low and there were few reports of injection-site or allergic reactions, which were thoroughly monitored. In addition, the rates of ALT and creatine kinase >3×ULN were low.

In this study in patients with hypercholesterolemia at high or very-high CV risk, adding mAb316P to rosuvastatin 10 or 20 mg resulted in additional LDL-C lowering compared with the addition of ezetimibe or doubling the statin dose.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1-12. (canceled)

13. A therapeutic method comprising: (a) selecting a patient who is on a moderate-dose statin therapy and who exhibits a serum low-density lipoprotein cholesterol (LDL-C) level of greater than about 70 mg/dL after at least four weeks of receiving the moderate-dose statin therapy; and (b) administering to the patient one or more doses of a PCSK9 inhibitor in combination with the moderate-dose statin therapy.

14. The method of claim 13, wherein the moderate-dose statin therapy comprises a daily dose of about 20 mg to about 40 mg of atorvastatin.

15. The method of claim 13, wherein the moderate-dose statin therapy comprises a daily dose of about 10 mg to about 20 mg of rosuvastatin.

16. The therapeutic method of claim 13, wherein the patient exhibits a serum LDL-C level of greater than about 100 mg/dL.

17. The therapeutic method of claim 13, wherein the patient is further selected on the basis of exhibiting one or more characteristic(s) selected from the group consisting of: (a) heterozygous Familial Hypercholesterolemia (heFH); (b) non-heterozygous Familial Hypercholesterolemia (non-FH); (c) a history of documented coronary heart disease (CHD); (d) non-coronary heart disease-cardiovascular disease (non-CHD CVD); (e) diabetes mellitus with target organ damage; (f) diabetes mellitus without target organ damage; (g) a calculated 10-year fatal cardiovascular disease risk SCORE greater than or equal to 5%; and (h) moderate chronic kidney disease.

18. The method of claim 13, wherein the PCSK9 inhibitor is an antibody or antigen-binding fragment thereof that specifically binds PCSK9.

19. The method of claim 18, wherein the antibody or antigen-binding protein that specifically binds PCSK9 is administered to the patient at a dose of about 75 mg at a frequency of once every two weeks.

20. The method of claim 18, wherein the antibody or antigen-binding protein that specifically binds PCSK9 is administered to the patient at a dose of about 150 mg at a frequency of once every two weeks.

21. The method of claim 18, wherein the antibody or antigen binding fragment thereof comprises the heavy and light chain CDRs of a HCVR/LCVR amino acid sequence pair comprising SEQ ID NOs: 1/6.

22. The method of claim 21, wherein the antibody or antigen-binding fragment thereof comprises heavy and light chain CDR amino acid sequences having SEQ ID NOs:2, 3, 4, 7, 8 and 10.

23. The method of claim 22, wherein the antibody or antigen-binding fragment thereof comprises an HCVR having the amino acid sequence of SEQ ID NO:1 and an LCVR having the amino acid sequence of SEQ ID NO:6.

24. The method of claim 18, wherein the antibody or antigen-binding fragment thereof binds to the same epitope on PCSK9 as an antibody comprising heavy and light chain CDR amino acid sequences having SEQ ID NOs:2, 3, 4, 7, 8 and 10.

25. The method of claim 18, wherein the antibody or antigen-binding fragment thereof competes for binding to PCSK9 with an antibody comprising heavy and light chain CDR amino acid sequences having SEQ ID NOs:2, 3, 4, 7, 8 and 10.

26. A therapeutic method comprising: (a) selecting a patient who is on a moderate-dose statin therapy and who exhibits a serum low-density lipoprotein cholesterol (LDL-C) level of greater than about 70 mg/dL after at least four weeks of receiving the moderate-dose statin therapy; (b) administering to the patient one or more initial doses of a pharmaceutical composition comprising 75 mg of an antibody or antigen-binding fragment thereof that specifically binds hPCSK9 (“the 75 mg doses”) in combination with the moderate-dose statin therapy; and (c) if the patient has not achieved a serum LDL-C level of less than 70 mg/dL following administration of one or more of the 75 mg doses, then: (i) discontinuing administration of the 75 mg doses; and (ii) administering to the patient one or more additional doses of a pharmaceutical composition comprising 150 mg of the antibody or antigen-binding fragment thereof that specifically binds hPCSK9 (“the 150 mg doses”); wherein each dose of antibody or antigen-binding fragment thereof is administered to the patient once every two weeks.

27-30. (canceled)

31. The method of claim 26, wherein the antibody or antigen binding fragment thereof comprises the heavy and light chain CDRs of a HCVR/LCVR amino acid sequence pair comprising SEQ ID NOs: 1/6.

32. The method of claim 31, wherein the antibody or antigen-binding fragment thereof comprises heavy and light chain CDR amino acid sequences having SEQ ID NOs:2, 3, 4, 7, 8 and 10.

33. The method of claim 32, wherein the antibody or antigen-binding fragment thereof comprises an HCVR having the amino acid sequence of SEQ ID NO:1 and an LCVR having the amino acid sequence of SEQ ID NO:6.

34. The method of claim 26, wherein the antibody or antigen-binding fragment thereof binds to the same epitope on PCSK9 as an antibody comprising heavy and light chain CDR amino acid sequences having SEQ ID NOs:2, 3, 4, 7, 8 and 10.

35. The method of claim 26, wherein the antibody or antigen-binding fragment thereof competes for binding to PCSK9 with an antibody comprising heavy and light chain CDR amino acid sequences having SEQ ID NOs:2, 3, 4, 7, 8 and 10.
